SEQUENCE LISTING (13 sequences)

SEQ ID NO: 1 - DNA, 747 bp, Homo sapiens
  source 1..747: mol_type = genomic DNA
atgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataa

SEQ ID NO: 2 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 3 - DNA, 6713 bp, synthetic construct
  source 1..6713: mol_type = other DNA
atcacctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagcttgcctatataagcagagctcgtttagtgaaccgtcagatcgcctggagacgccatccacgctgttttgacctccatagaagacaccgggaccgatccagcctccgcggtggcggcatcggtacacaagtttgtacaaaaaagcaggcttagccaccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataatacccagctttcttgtacaaagtggtatacccatacgatgttccagattacgcttagtaatgagaaatactcagaagatgtcactcaccatagtccgcagtgaaagataaatgatcgctgcgtttgatgagtttgtgttttagagctagaaatagcaagtaggccactgagcaagaactatgaatgagatctttagacatattgcggccgctttctccgcgatccagacatgataagatacattgatgagtttggacaaaccaaaactagaatgcagtgaaaaaaatgccttatttgtgaaatttgtgatgctattgccttatttgtaaccattataagctgcaataaacaagttatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccatgtctccgggagctgcatgtgtcagaggttttcaccgtcatcaccgaaacacgcgagttcgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttcacgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggatgagcgcccaatacgcaaaccgcctctcaccgcgcgttggccgattcattaatgcagctggcacgacaggtttcccgactggaaagcgggcagtgagcgcaacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaggtcgacttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttagcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagattcatgcgccaggttttctggtcatgcaccacgttcgtgggccctcaggaacctccacgtcagcggtaactgtgaagcccagtcgctcgtaaaaaggaagattccggggagcactggtttcgaggaacgccggcacgccagcccgttccgcggcctcaacaccaggaagaacgacagcgcttcccaaacctttaccttggtgatctgggctgacaccgacagtggcaagaaaccacgcaggctcctttggtctatgaggagccagcaagccctccatttgctgttgtgccgccaacctactccctgacagctcggccatccgcggacctatttccgcgaacactgcgccggcttcgacgctttccggagtagtccagaccgcaactgcggcaccatcgtccgcaacccaaactttaccaatgtccagacccacgcgcgtaagaaacagctcttgcaactcagttaccctctctatgtgtctgtccggatctacggtatggcgggtcgctggataatctgcaaatgctgcggcaagtgttcggacagcccgtggtacatcgtcacgggtagcgagcctaacagtgggcttatattccgtcatggggccggggttctcctccacgtcgccggcctgcttcagcaggctgaagttggtggcgccgctgcccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcacgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatcttatcgtcatcgtctttgtaatccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccactcgagtggaatt

SEQ ID NO: 4 - DNA, 3425 bp, synthetic construct
  source 1..3425: mol_type = other DNA
gcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctggagattcatgcgccaggttttctggtcatgcaccacgttcgtgggccctcaggaacctccacgtcagcggtaactgtgaagcccagtcgctcgtaaaaaggaagattccggggagcactggtttcgaggaacgccggcacgccagcccgttccgcggcctcaacaccaggaagaacgacagcgcttcccaaacctttaccttggtgatctgggctgacaccgacagtggcaagaaaccacgcaggctcctttggtctatgaggagccagcaagccctccatttgctgttgtgccgccaacctactccctgacagctcggccatccgcggacctatttccgcgaacactgcgccggcttcgacgctttccggagtagtccagaccgcaactgcggcaccatcgtccgcaacccaaactttaccaatgtccagacccacgcgcgtaagaaacagctcttgcaactcagttaccctctctatgtgtctgtccggatctacggtatggcgggtcgctggataatctgcaaatgctgcggcaagtgttcggacagcccgtggtacatcgtcacgggtagcgagcctaacagtgggcttatattccgtcatggggccggggttctcctccacgtcgccggcctgcttcagcaggctgaagttggtggcgccgctgcccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcacgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatcttatcgtcatcgtctttgtaatccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccactcgagtggaattatcacctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagcttgcctatataagcagagctcgtttagtgaaccgtcagatcgcctggagacgccatccacgctgttttgacctccatagaagacaccgggaccgatccagcctccgcggtggcggcatcggtacacaagtttgtacaaaaaagcaggcttagccaccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggcggagctccccgaggtcgctatggccccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataatacccagctttcttgtacaaagtggtatacccatacgatgttccagattacgcttagtaatgagaaatactcagaagatgtcactcaccatagtccgcagtgaaagataaatgatcgctgcgtttgatgagtttgtgttttagagctagaaatagcaagtaggccactgagcaagaactatgaatgagatctttagacatattgcggccgctttctccgcgatccagacatgataagatacattgatgagtttggacaaaccaaaactagaatgcagtgaaaaaaatgccttatttgtgaaatttgtgatgctattgccttatttgtaaccattataagctgcaataaacaagtt

SEQ ID NO: 5 - protein, 648 aa, Homo sapiens
MSRKASENVEYTLRSLSSLMGERRRKQPEPDAASAAGECSLLAAAESSTSLQSAGAGGGGVGDLERAARRQFQQDETPAFVYVVAVFSALGGFLFGYDTGVVSGAMLLLKRQLSLDALWQELLVSSTVGAAAVSALAGGALNGVFGRRAAILLASALFTAGSAVLAAANNKETLLAGRLVVGLGIGIASMTVPVYIAEVSPPNLRGRLVTINTLFITGGQFFASVVDGAFSYLQKDGWRYMLGLAAVPAVIQFFGFLFLPESPRWLIQKGQTQKARRILSQMRGNQTIDEEYDSIKNNIEEEEKEVGSAGPVICRMLSYPPTRRALIVGCGLQMFQQLSGINTIMYYSATILQMSGVEDDRLAIWLASVTAFTNFIFTLVGVWLVEKVGRRKLTFGSLAGTTVALIILALGFVLSAQVSPRITFKPIAPSGQNATCTRYSYCNECMLDPDCGFCYKMNKSTVIDSSCVPVNKASTNEAAWGRCENETKFKTEDIFWAYNFCPTPYSWTALLGLILYLVFFAPGMGPMPWTVNSEIYPLWARSTGNACSSGINWIFNVLVSLTFLHTAEYLTYYGAFFLYAGFAAVGLLFIYGCLPETKGKKLEEIESLFDNRLCTCGTSDSDEGRYIEYIRVKGSNYHLSDNDASDVE

SEQ ID NO: 6 - protein, 741 aa, Homo sapiens
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 7 - protein, 676 aa, Homo sapiens
MAKVNITRDLIRRQIKERGALSFERRYHVTDPFIRRLGLEAELQGHSGCVNCLEWNEKGDLLASGSDDQHTIVWDPLHHKKLLSMHTGHTANIFSVKFLPHAGDRILITGAADSKVHVHDLTVKETIHMFGDHTNRVKRIATAPMWPNTFWSAAEDGLIRQYDLRENSKHSEVLIDLTEYCGQLVEAKCLTVNPQDNNCLAVGASGPFVRLYDIRMIHNHRKSMKQSPSAGVHTFCDRQKPLPDGAAQYYVAGHLPVKLPDYNNRLRVLVATYVTFSPNGTELLVNMGGEQVYLFDLTYKQRPYTFLLPRKCHSSGVQNGKMSTNGVSNGVSNGLHLHSNGFRLPESRGHVSPQVELPPYLERVKQQANEAFACQQWTQAIQLYSKAVQRAPHNAMLYGNRAAAYMKRKWDGDHYDALRDCLKAISLNPCHLKAHFRLARCLFELKYVAEALECLDDFKGKFPEQAHSSACDALGRDITAALFSKNDGEEKKGPGGGAPVRLRSTSRKDSISEDEMVLRERSYDYQFRYCGHCNTTTDIKEANFFGSNAQYIVSGSDDGSFFIWEKETTNLVRVLQGDESIVNCLQPHPSYCFLATSGIDPVVRLWNPRPESEDLTGRVVEDMEGASQANQRRMNADPLEVMLLNMGYRITGLSSGGAGASDDEDSSEGQVQCRPS

SEQ ID NO: 8 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 9 - protein, 611 aa, Homo sapiens
MPRRKRNAGSSSDGTEDSDFSTDLEHTDSSESDGTSRRSARVTRSSARLSQSSQDSSPVRNLQSFGTEEPAYSTRRVTRSQQQPTPVTPKKYPLRQTRSSGSETEQVVDFSDRETKNTADHDESPPRTPTGNAPSSESDIDISSPNVSHDESIAKDMSLKDSGSDLSHRPKRRRFHESYNFNMKCPTPGCNSLGHLTGKHERHFSISGCPLYHNLSADECKVRAQSRDKQIEERMLSHRQDDNNRHATRHQAPTERQLRYKEKVAELRKKRNSGLSKEQKEKYMEHRQTYGNTREPLLENLTSEYDLDLFRRAQARASEDLEKLRLQGQITEGSNMIKTIAFGRYELDTWYHSPYPEEYARLGRLYMCEFCLKYMKSQTILRRHMAKCVWKHPPGDEIYRKGSISVFEVDGKKNKIYCQNLCLLAKLFLDHKTLYYDVEPFLFYVMTEADNTGCHLIGYFSKEKNSFLNYNVSCILTMPQYMRQGYGKMLIDFSYLLSKVEEKVGSPERPLSDLGLISYRSYWKEVLLRYLHNFQGKEISIKEISQETAVNPVDIVSTLQALQMLKYWKGKHLVLKRQDLIDEWIAKEAKRSNSNKTMDPSCLKWTPPKGT

SEQ ID NO: 10 - protein, 595 aa, Homo sapiens [sequence omitted]

SEQ ID NO: 11 - protein, 816 aa, Homo sapiens
MAEPLKEEDGEDGSAEPPGPVKAEPAHTAASVAAKNLALLKARSFDVTFDVGDEYEIIETIGNGAYGVVSSARRRLTGQQVAIKKIPNAFDVVTNAKRTLRELKILKHFKHDNIIAIKDILRPTVPYGEFKSVYVVLDLMESDLHQIIHSSQPLTLEHVRYFLYQLLRGLKYMHSAQVIHRDLKPSNLLVNENCELKIGDFGMARGLCTSPAEHQYFMTEYVATRWYRAPELMLSLHEYTQAIDLWSVGCIFGEMLARRQLFPGKNYVHQLQLIMMVLGTPSPAVIQAVGAERVRAYIQSLPPRQPVPWETVYPGADRQALSLLGRMLRFEPSARISAAAALRHPFLAKYHDPDDEPDCAPPFDFAFDREALTRERIKEAIVAEIEDFHARREGIRQQIRFQPSLQPVASEPGCPDVEMPSPWAPSGDCAMESPPPAPPPCPGPAPDTIDLTLQPPPPVSEPAPPKKDGAISDNTKAALKAALLKSLRSRLRDGPSAPLEAPEPRKPVTAQERQREREEKRRRRQERAKEREKRRQERERKERGAGASGGPSTDPLAGLVLSDNDRSLLERWTRMARPAAPALTSVPAPAPAPTPTPTPVQPTSPPPGPVAQPTGPQPQSAGSTSGPVPQPACPPPGPAPHPTGPPGPIPVPAPPQIATSTSLLAAQSLVPPPGLPGSSTPGVLPYFPPGLPPPDAGGAPQSSMSESPDVNLVTQQLSKSQVEDPLPPVFSGTPKGSGAGYGVGFDLEEFLNQSFDMGVADGPQDGQADSASLSASLLADWLEGHGMNPADIESLQREIQMDSPMLLADLPDLQDP

SEQ ID NO: 12 - protein, 1401 aa, Homo sapiens
MKSCGVSLATAAAAAAAFGDEEKKMAAGKASGESEEASPSLTAEEREALGGLDSRLFGFVRFHEDGARTKALLGKAVRCYESLILKAEGKVESDFFCQLGHFNLLLEDYPKALSAYQRYYSLQSDYWKNAAFLYGLGLVYFHYNAFQWAIKAFQEVLYVDPSFCRAKEIHLRLGLMFKVNTDYESSLKHFQLALVDCNPCTLSNAEIQFHIAHLYETQRKYHSAKEAYEQLLQTENLSAQVKATVLQQLGWMHHTVDLLGDKATKESYAIQYLQKSLEADPNSGQSWYFLGRCYSSIGKVQDAFISYRQSIDKSEASADTWCSIGVLYQQQNQPMDALQAYICAVQLDHGHAAAWMDLGTLYESCNQPQDAIKCYLNATRSKSCSNTSALAARIKYLQAQLCNLPQGSLQNKTKLLPSIEEAWSLPIPAELTSRQGAMNTAQQNTSDNWSGGHAVSHPPVQQQAHSWCLTPQKLQHLEQLRANRNNLNPAQKLMLEQLESQFVLMQQHQMRPTGVAQVRSTGIPNGPTADSSLPTNSVSGQQPQLALTRVPSVSQPGVRPACPGQPLANGPFSAGHVPCSTSRTLGSTDTILIGNNHITGSGSNGNVPYLQRNALTLPHNRTNLTSSAEEPWKNQLSNSTQGLHKGQSSHSAGPNGERPLSSTGPSQHLQAAGSGIQNQNGHPTLPSNSVTQGAALNHLSSHTATSGGQQGITLTKESKPSGNILTVPETSRHTGETPNSTASVEGLPNHVHQMTADAVCSPSHGDSKSPGLLSSDNPQLSALLMGKANNNVGTGTCDKVNNIHPAVHTKTDNSVASSPSSAISTATPSPKSTEQTTTNSVTSLNSPHSGLHTINGEGMEESQSPMKTDLLLVNHKPSPQIIPSMSVSIYPSSAEVLKACRNLGKNGLSNSSILLDKCPPPRPPSSPYPPLPKDKLNPPTPSIYLENKRDAFFPPLHQFCTNPNNPVTVIRGLAGALKLDLGLFSTKTLVEANNEHMVEVRTQLLQPADENWDPTGTKKIWHCESNRSHTTIAKYAQYQASSFQESLREENEKRSHHKDHSDSESTSSDNSGRRRKGPFKTIKFGTNIDLSDDKKWKLQLHELTKLPAFVRVVSAGNLLSHVGHTILGMNTVQLYMKVPGSRTPGHQENNNFCSVNINIGPGDCEWFVVPEGYWGVLNDFCEKNNLNFLMGSWWPNLEDLYEANVPVYRFIQRPGDLVWINAGTVHWVQAIGWCNNIAWNVGPLTACQYKLAVERYEWNKLQSVKSIVPMVHLSWNMARNIKVSDPKLFEMIKYCLLRTLKQCQTLREALIAAGKEIIWHGRTKEEPAHYCSICEVEVFDLLFVTNESNSRKTYIVHCQDCARKTSGNLENFVVLEQYKMEDLMQVYDQFTLAPPLPSASS

SEQ ID NO: 13 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

(19) United States
(12) Patent Application Publication
(10) Pub. No.: US 2025/0313599 A1
(43) Pub. Date: Oct. 9, 2025

(54) TREATMENTS FOR AGE-RELATED CELLULAR DYSFUNCTION

(71) Applicants: President and Fellows of Harvard College, Cambridge, MA (US); CENTRO DE INVESTIGACION COOPERATIVA EN BIOCIENCIAS (CIC bioGUNE), Derio (ES); University of Luxembourg, Esch-sur-Alzette (LU)

(72) Inventors: Alexandru Plesa, Cambridge, MA (US); Sascha Jung, Cambridge, MA (US); George M. Church, Cambridge, MA (US); Antonio Del Sol Mesa, Cambridge, MA (US); Helen Wang, Cambridge, MA (US); Michael Shadpour, Cambridge, MA (US)

(73) Assignees: President and Fellows of Harvard College, Cambridge, MA (US); CENTRO DE INVESTIGACION COOPERATIVA EN BIOCIENCIAS (CIC bioGUNE), Derio (ES); University of Luxembourg, Esch-sur-Alzette (LU)

(21) Appl. No.: 18/862,831

(22) PCT Filed: May 5, 2023
(86) PCT No.: PCT/US2023/066647

§ 371 Date: Nov. 4, 2024

Related U.S. Application Data
(60) Provisional application No. 63/338,967, filed on May 6, 2022.
(60) Provisional application No. 63/357,207, filed on Jun. 30, 2022.
(60) Provisional application No. 63/413,818, filed on Oct. 6, 2022.
(60) Provisional application No. 63/423,430, filed on Nov. 7, 2022.

(51) Int. Cl. C07K 14/47; A61K 38/17; A61K 48/00; C12N 5/077

(52) U.S. Cl. CPC C07K 14/47; A61K 38/1709; A61K 48/005; C12N 5/0656; C12N 2510/00

(57) ABSTRACT

The disclosure relates to a method of inducing cellular rejuvenation of a cell comprising contacting the cell with an effective amount of a SRSF1, SLC2A13, RNASEL, WDTC1, and/or NPM1 protein or a nucleic acid encoding the SRSF1, SLC2A13, RNASEL, WDTC1, and/or NPM1 protein. The disclosure also relates to a method of inducing cellular rejuvenation of a cell comprising contacting the cell with an effective amount of an inhibitor of KAT7, ESR1, MAPK7, KDM6A, and/or CTNNB1 protein expression or expression of a nucleic acid encoding a KAT7, ESR1, MAPK7, KDM6A, and/or CTNNB1 protein.

### CROSS-REFERENCE TO RELATED APPLICATIONS

This application claims the benefit under 35 U.S.C. § 119(e) of U.S. Provisional Application No. 63/338,967, filed May 6, 2022, U.S. Provisional Application No. 63/357,207, filed Jun. 30, 2022, U.S. Provisional Application No. 63/413,818, filed Oct. 6, 2022, and U.S. Provisional Application No. 63/423,430, filed Nov. 7, 2022, each of which is hereby incorporated by reference in its entirety.

### REFERENCE TO AN ELECTRONIC SEQUENCE LISTING

The contents of the electronic sequence listing (H049870765WO00-SEQ-HJD; Size: 29,862 bytes; and Date of Creation: Apr. 28, 2023) is hereby incorporated by reference in its entirety.

### BACKGROUND

Aging is characterized by a gradual loss of function occurring at the molecular, cellular, tissue and organismal levels. At the chromatin level, aging is associated with the progressive accumulation of epigenetic errors that eventually lead to aberrant gene regulation, stem cell exhaustion, senescence, and deregulated cell/tissue homeostasis. The technology of nuclear reprogramming to pluripotency, through over-expression of a small number of transcription factors, can revert both the age and the identity of any cell to that of an embryonic cell by driving epigenetic reprogramming. The undesirable erasure of cell identity is problematical for the development of rejuvenation therapies because of the resulting destruction of the structure, function and cell type distribution in tissues and organs.

### SUMMARY

Aspects of the present disclosure relate to a genetic intervention for reversing the age of cells, such as human dermal fibroblasts. In some embodiments, expression of the Serine and Arginine Rich Splicing Factor 1 (SRSF1) protein reverses the transcriptomic age of the fibroblast. In some embodiments, expression of the Solute Carrier Family 2 Member 13 (SLC2A13) protein reverses the transcriptomic age of the fibroblast. In some embodiments, expression of the 2-5A-dependent ribonuclease (RNASEL) protein reverses the transcriptomic age of the fibroblast. In some embodiments, expression of the WD and tetratricopeptide repeats protein 1 (WDTC1) protein reverses the transcriptomic age of the fibroblast. In some embodiments, expression of nucleophosmin (NPM1) protein reverses the transcriptomic age of the fibroblast.

In some embodiments, reversing the age of cells is accomplished by inhibiting the expression of one or more pro-aging proteins. The one or more pro-aging proteins may be selected from the group consisting of: KAT7, ESR1, MAPK7, KDM6A, and CTNNB1. In some embodiments, reversing the age of cells is accomplished by inhibiting the expression of KAT7. In some embodiments, reversing the age of cells is accomplished by inhibiting the expression of ESR1. In some embodiments, reversing the age of cells is accomplished by inhibiting the expression of MAPK7. In some embodiments, reversing the age of cells is accomplished by inhibiting the expression of KDM6A. In some embodiments, reversing the age of cells is accomplished by inhibiting the expression of CTNNB1.

Some aspects provide a method of inducing cellular rejuvenation of a cell comprising contacting the cell with an effective amount of a SRSF1 protein or a nucleic acid encoding the SRSF1 protein. Some aspects provide a method of inducing cellular rejuvenation of a cell comprising contacting the cell with an effective amount of a SLC2A13 protein or a nucleic acid encoding the SLC2A13 protein. Some aspects provide a method of inducing cellular rejuvenation of a cell comprising contacting the cell with an effective amount of a RNASEL protein or a nucleic acid encoding the RNASEL protein. Some aspects provide a method of inducing cellular rejuvenation of a cell comprising contacting the cell with an effective amount of a WDTC1 protein or a nucleic acid encoding the WDTC1 protein. Some aspects provide a method of inducing cellular rejuvenation of a cell comprising contacting the cell with an effective amount of an NMP1 protein or a nucleic acid encoding the NPM1 protein.

Some aspects provide a method of inducing cellular rejuvenation of a cell comprising contacting the cell with an effective amount of an inhibitor of expression of one or more pro-aging proteins. In some embodiments, the one or more pro-aging proteins is selected from the group consisting of: KAT7, ESR1, MAPK7, KDM6A, and CTNNB1. Some aspects provide a method of inducing cellular rejuvenation of a cell comprising contacting the cell with an effective amount of an inhibitor of expression of KAT7. Some aspects provide a method of inducing cellular rejuvenation of a cell comprising contacting the cell with an effective amount of an inhibitor of expression of ESR1. Some aspects provide a method of inducing cellular rejuvenation of a cell comprising contacting the cell with an effective amount of an inhibitor of expression of MAPK7. Some aspects provide a method of inducing cellular rejuvenation of a cell comprising contacting the cell with an effective amount of an inhibitor of expression of KDM6A. Some aspects provide a method of inducing cellular rejuvenation of a cell comprising contacting the cell with an effective amount of an inhibitor of expression of CTNNB1.

Other aspects provide a method of inducing cellular rejuvenation of a cell in a subject comprising administering to the subject an effective amount of a SRSF1 protein or a nucleic acid encoding the SRSF1 protein. Other aspects provide a method of inducing cellular rejuvenation of a cell in a subject comprising administering to the subject an effective amount of a SLC2A13 protein or a nucleic acid encoding the SLC2A13 protein. Other aspects provide a method of inducing cellular rejuvenation of a cell in a subject comprising administering to the subject an effective amount of a RNASEL protein or a nucleic acid encoding the RNASEL protein. Other aspects provide a method of inducing cellular rejuvenation of a cell in a subject comprising administering to the subject an effective amount of a WDTC1 protein or a nucleic acid encoding the WDTC1 protein. Other aspects provide a method of inducing cellular rejuvenation of a cell in a subject comprising administering to the subject an effective amount of an NPM1 protein or a nucleic acid encoding the NPM1 protein.

Other aspects provide a method of inducing cellular rejuvenation of a cell in a subject comprising administering to the subject an effective amount of an inhibitor of expression of one or more pro-aging proteins. In some embodiments, the one or more pro-aging proteins is selected from the group consisting of: KAT7, ESR1, MAPK7, KDM6A, and CTNNB1. Other aspects provide a method of inducing cellular rejuvenation of a cell in a subject comprising administering to the subject an effective amount of an inhibitor of expression of KAT7. Other aspects provide a method of inducing cellular rejuvenation of a cell in a subject comprising administering to the subject an effective amount of an inhibitor of expression of ESR1. Other aspects provide a method of inducing cellular rejuvenation of a cell in a subject comprising administering to the subject an effective amount of an inhibitor of expression of MAPK7. Other aspects provide a method of inducing cellular rejuvenation of a cell in a subject comprising administering to the subject an effective amount of an inhibitor of expression of KDM6A. Other aspects provide a method of inducing cellular rejuvenation of a cell in a subject comprising administering to the subject an effective amount of an inhibitor of expression of CTNNB1.

In some embodiments, the effective amount is sufficient to decrease cellular senescence of the cell, relative to a control. In some embodiments, the effective amount is sufficient to decrease cellular senescence by at least 20%, at least 30%, at least 40%, or at least 50%, relative to a control. In some embodiments, the effective amount is sufficient to decrease cellular senescence by at least 50%.

In some embodiments, the effective amount is sufficient to decrease senescence-associated β-galactosidase activity, relative to a control. For example, the effective amount may be sufficient to decrease the senescence-associated β-galactosidase activity by at least 25%, at least 30%, at least 35%, at least 40%, at least 45%, or at least 50%.

In some embodiments, the effective amount is sufficient to decrease proteasomal activity, relative to a control. For example, the effective amount may be sufficient to decrease the proteasomal activity by at least 25%, at least 30%, at least 35%, at least 40%, at least 45%, or at least 50%.

In some embodiments, the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years. In some embodiments, the effective amount is sufficient to induce an average cellular rejuvenation of about 5 years to about 50 years, or about 5 years to about 25 years. In some embodiments, the effective amount is sufficient to induce an average cellular rejuvenation of at least 25 years.

In some embodiments, the cells are selected from fibroblasts, hematopoietic stem cells, endothelial cells, chondrocytes, skeletal muscle stem cells, keratinocytes, mesenchymal stem cells and corneal epithelial cells. In some embodiments, the cells are fibroblasts, for example, human fibroblasts (e.g., from skin, bladder, lung, or the reproductive system). In some embodiments, the fibroblasts are human dermal fibroblasts. In some embodiments, the cells are stem cells, for example, hematopoietic stem cells (HSCs), such as human HSCs. In some embodiments, the cells are cardiomyocytes. In some embodiments, the cells are skeletal muscle stem cells.

In some embodiments, the nucleic acid comprises a heterologous promoter operably linked to the open reading frame. The heterologous promoter may be, for example, an inducible promoter.

In some embodiments, a method comprises delivering to cells the SRSF1 protein. In some embodiments, a method comprises delivering to cells the SLC2A13 protein. In some embodiments, a method comprises delivering to cells the RNASEL protein. In some embodiments, a method comprises delivering to cells the WDTC1 protein. In some embodiments, a method comprises delivering to cells the NPM1 protein.

In some embodiments, a method comprises delivering to cells an inhibitor of expression of one or more pro-aging proteins, for example, selected from the group consisting of: KAT7, ESR1, MAPK7, KDM6A, and CTNNB1. In some embodiments, a method comprises delivering to cells an inhibitor of expression of the KAT7 protein. In some embodiments, a method comprises delivering to cells an inhibitor of expression of the ESR1 protein. In some embodiments, a method comprises delivering to cells an inhibitor of expression of the MAPK7 protein. In some embodiments, a method comprises delivering to cells an inhibitor of expression of the KDM6A protein. In some embodiments, a method comprises delivering to cells an inhibitor of expression of the CTNNB1 protein.

In some embodiments, a method comprises delivering to cells the nucleic acid comprising an open reading frame encoding the SRSF1 protein. In some embodiments, a method comprises delivering to cells the nucleic acid comprising an open reading frame encoding the SLC2A13protein protein. In some embodiments, a method comprises delivering to cells the nucleic acid comprising an open reading frame encoding the RNASEL protein. In some embodiments, a method comprises delivering to cells the nucleic acid comprising an open reading frame encoding the WDTC1 protein. In some embodiments, a method comprises delivering to cells the nucleic acid comprising an open reading frame encoding the NPM1 protein.

In some embodiments, the nucleic acid is delivered on a non-viral vector (e.g., mRNA delivery via lipid nanoparticle (LNP), or electroporation). In other embodiments, the nucleic acid is delivered on a viral vector (e.g., adeno-associated viral (AAV) vector).

In some embodiments, the contacting comprises transfecting the cells.

Some aspects provide a method of inducing cellular rejuvenation of a cell comprising overexpressing in the cell an effective amount of a SRSF1 protein. In some embodiments, the method comprises activating expression or activity of endogenous SRSF1 protein at a level that is higher than a baseline level.

Some aspects provide a method of inducing cellular rejuvenation of a cell comprising overexpressing in the cell an effective amount of a SLC2A13 protein. In some embodiments, the method comprises activating expression or activity of endogenous SLC2A13 protein at a level that is higher than a baseline level.

Some aspects provide a method of inducing cellular rejuvenation of a cell comprising overexpressing in the cell an effective amount of a RNASEL protein. In some embodiments, the method comprises activating expression or activity of endogenous RNASEL protein at a level that is higher than a baseline level.

Some aspects provide a method of inducing cellular rejuvenation of a cell comprising overexpressing in the cell an effective amount of a WDTC1 protein. In some embodiments, the method comprises activating expression or activity of endogenous WDTC1 protein at a level that is higher than a baseline level.

Some aspects provide a method of inducing cellular rejuvenation of a cell comprising overexpressing in the cell an effective amount of an NPM1 protein. In some embodiments, the method comprises activating expression or activity of endogenous NPM1 protein at a level that is higher than a baseline level.

Some aspects provide a method of inducing cellular rejuvenation of a cell comprising delivering to the cell an effective amount of an inhibitor of expression of one or more pro-aging proteins. For example, the one or more pro-aging proteins may be selected from the group consisting of: KAT7, ESR1, MAPK7, KDM6A, and CTNNB1. Some aspects provide a method of inducing cellular rejuvenation of a cell comprising delivering to the cell an effective amount of an inhibitor of expression of KAT7. Some aspects provide a method of inducing cellular rejuvenation of a cell comprising delivering to the cell an effective amount of an inhibitor of expression of ESR1. Some aspects provide a method of inducing cellular rejuvenation of a cell comprising delivering to the cell an effective amount of an inhibitor of expression of MAPK7. Some aspects provide a method of inducing cellular rejuvenation of a cell comprising delivering to the cell an effective amount of an inhibitor of expression of KDM6A. Some aspects provide a method of inducing cellular rejuvenation of a cell comprising delivering to the cell an effective amount of an inhibitor of expression of CTNNB1.

In some embodiments, the method comprises reducing expression or activity of one or more endogenous pro-aging proteins at a level lower than a baseline level. In some embodiments, the method comprises reducing expression or activity of KAT7 at a level lower than a baseline level. In some embodiments, the method comprises reducing expression or activity of ESR1at a level lower than a baseline level. In some embodiments, the method comprises reducing expression or activity of ESR1at a level lower than a baseline level. In some embodiments, the method comprises reducing expression or activity of MAPK7at a level lower than a baseline level. In some embodiments, the method comprises reducing expression or activity of CTNNB1 at a level lower than a baseline level.

Some aspects provide a cell comprising an engineered nucleic acid encoding a SRSF1 protein.

In some embodiments, the cell is a fibroblast.

In some embodiments, the fibroblast is a human dermal fibroblast.

In some embodiments, the cell is a stem cell.

In some embodiments, the stem cell is selected from hematopoietic stem cells, skeletal muscle stem cells, and mesenchymal stem cells.

In some embodiments, the stem cell is a human induced pluripotent stem cell.

In some embodiments, the cell is selected from endothelial cells, chondrocytes, keratinocytes, and corneal epithelial cells.

In some embodiments, the cell expresses SRSF1 at a level that is higher than a baseline level.

### BRIEF DESCRIPTION OF THE DRAWINGS

FIGS. 1A-1E. Process-based transcriptomic clock reports on biological age of human fibroblasts. FIG. 1A: Schematic for transcriptomic clock training. Longitudinal transcriptomic data from human fibroblasts is used to train process-specific weak age predictors, which are then combined into an ensemble predictor. FIG. 1B: Aging score for different clock processes across the human lifespan. Score is calculated as a weighted average of process specific clock gene expression and normalized across all ages (n=133), such as the highest score is seen in the oldest samples. The processes are split between three clusters based on their trajectories. Histone methylation experiences an early logarithmic increase; junction organization, lipid transport, and sodium transport have a middle-age inflection point; actin polymerization, ERQC, translation initiation, and WNT signaling exhibit a late exponential increase with age. FIG. 1C: Validation of the transcriptomic clock on human dermal fibroblasts from independent datasets (n=12). The RNA clock maintains high accuracy (Pearson's r=0.93, p-value <2.2e-16; MAE=6.66) across different sequencing platforms. FIG. 1D: Response of RNA clock to different age-modulating in vitro interventions. Galactose has no effect on the age of the cells, unlike 2-deoxy-D-glucose (2DG), dexamethasone (DEX) treatment and contact inhibition (CI), which showed significant increases in the predicted age. Growth in hypoxia (3% O₂) showed partial reversal of the detrimental effect of contact inhibition. FIG. 1E: Response of RNA clock and DNA methylation (DNAm) clock (Horvath Skin and Blood5) to cellular reprogramming time course. The predicted age is scaled across the entire time course and exhibits a steeper and faster decrease in the RNA compared to the DNAm clock. The two clocks reach a consensus at the iPS stage suggesting that the rejuvenation effect of reprogramming peaks after 7 days of reprogramming, in line with previous studies

FIGS. 2A-2D. Age reversal screen identifies genes for cellular rejuvenation. FIG. 2A: Workflow of cDNA overexpression screen for rejuvenating interventions. 95 different plasmids containing the gene of interest in an overexpression cassette along with a piggyBac transposase expressing plasmid were electroporated into 3 different NHDF lines. The edited fibroblasts were induced using Dox (1 ug/mL) for 3 days and assayed for age-related changes using RNA-Seq and flow cytometry assays. FIG. 2B: Heatmap of gene induction over all the overexpression lines in the three different NHDF backgrounds (n=2). There is a consensus across the log 2FoldChange levels across the 3 different lines, and a range of induction values across all tested genes were observed. FIG. 2C: Z-score for the age effect, as measured by our RNA clock, in the overexpression screen across the three NHDF lines (n=2). Genes with a z-score lower than-1 (their age lowering effect is more than 1 standard deviation away from the mean), KAT7 (positive control for pro-aging effect), and BFP (negative control) are labeled. FIG. 2D: Staining data for representative gene hits and controls across the three assayed cellular processes in the M55 and M79 lines (n=3). Data is normalized with respect to the gene specific No Doxycycline control and the line specific gene control, and significance is tested using the two-tailed Student's t-test.

FIGS. 3A-3E. Variability of the aging phenotype and its response to perturbations. FIG. 3A: Total transcriptome UMAP on overexpression lines from age reversal screen. There is a strong clustering based on donor cell lines with only a few perturbations overcoming line-specific differences. FIG. 3B: Relationship between transcriptomic variability of interventions across cell lines and their induced differentially expressed clock genes. A strong inverse correlation between the mean UMAP distance of biological replicates of the same gene and the associated number of clock DEGs were observed, with OSKM, NOTCH1, SRSF1, and KDM6A having the largest effects. FIG. 3C: 3D landscape of all 480 NHDF transcriptomes from the large-scale screen, with the x-y axis representing the UMAP coordinates from a, while the Z-axis represents the predicted age, with the youngest samples having the largest values. FIG. 3D: Heatmap of differential (older age-younger age) process activity in 7 pairs of isogenic cell lines. Higher activity is associated with an older phenotype, while lower activity is representative of young cells. Four distinct groups were observed based on the nature of the process dysfunction: group I=ERQC and lipid transport, group II =WNT signaling and sodium transport, group III=histone methylation, group IV=translation initiation and sodium transport. FIG. 3E: Scaled age effect of SIRT1 overexpression on the clock processes across six lines (n=2). Negative age effects represent younger gene expression profiles. No coordinated effects across the cell lines were observed, but it was noticed that the responder lines (M65, M67, M68, M79) all had moderate rejuvenation in WNT signaling.

FIGS. 4A-4D. SRSF1 induces robust cellular rejuvenation. FIG. 4A: Predicted age effect of hit overexpression in validation experiments using 6 NHDF lines (n=2). Robust age reduction in the SRSF1 samples was observed, and a beneficial effect of SIRT1 on the transcriptome in 4 out of 6 lines. The other 3 genes had weak cell-line specific effects with the exception of WDTC1, which showed marked rejuvenation in the M69 line. FIG. 4B: Staining data for SRSF1 across three assayed cellular processes in the six cell lines (n=3). Data is normalized with respect to the uninduced samples and is reported as a relative effect to the induced control, and significance is tested using the two-tailed Student's t-test. A robust significant reduction in the SA-βGal and proteasomal activity was observed, but no coordinated effect on the mitochondrial membrane potential. FIG. 4C: Scratch assay time course, expressed as wound area scaled to the 0 h time point, for the M79 line after the induction of OKSM, SRSF1, or BFP. Compared to the BFP control, a faster wound area closure in the SRSF1 treated cells and a slower response in those that were induced with OSKM was observed. Data is averaged over replicates (n=3-5), error bars represent standard error of the mean, and significance with respect to the BFP control is tested using the two-tailed Student's t-test. FIG. 4D: Scaled age effect of SRSF1 overexpression on the clock processes across six lines (n=2). Negative age effects represent younger gene expression profiles. High correlation between the process effects across all 6 lines was observed, with a significant improvement in histone methylation and translation initiation.

FIG. 5. Normalized expression of SRSF1 in primary NHDFs of different age groups. Expression difference between young and old group is statistically significant (two-tailed t-test p=0.002).

FIG. 6A-6B. FIG. 6A: SA-βGal activity in young (n=5) and old (n=5) NHDF lines. While there is a clear effect in the old samples, more replicates are needed to reach statistical significance. FIG. 6B: Relative effect of SRSF1 mRNA transfection on SA-βGal activity. Results are normalized to the negative transfection control and presented as percent difference in SA-BGal activity for 10 different lines aged: 17, 22, 25, 29, 30, 65, 67, 68, 69, and 79 years old. Overall, the effect of SRSF1 mRNA transfection significantly reduced the senescence phenotype (two-tailed t-test p=0.004) in NHDFs.

FIG. 7A-7B. FIG. 7A: Collagen expression in young (n=5) and old (n=5) NHDF lines. There is a statistically significant decrease in collagen production in the old cells (two-tailed t-test p=0.02). FIG. 7B: Relative effect of SRSF1 mRNA transfection on collagen production. Results are normalized to untreated control and presented as percent difference in collagen staining for 10 different lines aged: 17, 22, 25, 29, 30, 65, 67, 68, 69, and 79 years old. Overall, the effect of SRSF1 mRNA transfection significantly increased collagen production (two-tailed t-test p<0.001) in NHDFs.

FIG. 8. Left: ROS levels upon H2O2 treatment in young (n=5) and old (n=5) NHDF lines. There is a statistically significant increase in ROS levels in the old cells (two-tailed t-test p<0.001). Right: ROS levels upon H2O2 treatment in old (n=5) NHDF lines transfected with control or SRSF1 mRNA. Overall, the effect of SRSF1 mRNA transfection significantly reduced ROS levels after H2O2 treatment (two-tailed t-test p=0.002) in old NHDFs.

FIG. 9. Left: Experimental design schematic for in vitro scratch assay. Right: Scratch assay time course, expressed as wound area scaled to the 0 h time point, for the M79 line after the induction of OKSM, SRSF1, or BFP. Compared to the BFP control, we observed a faster wound area closure in the SRSF1 treated cells and a slower response in those that were induced with OSKM. Data is averaged over replicates (n=3-5), error bars represent standard error of the mean, and significance with respect to the BFP control is tested using the two-tailed Student's t-test.

FIG. 10. Left: Experimental design schematic for in vivo wound healing assay. Right: Excisional wound area over time, scaled to the day 1 time point, for old-GFP, old-SRSF1 (ON-ON), and young-GFP mice. Data are shown as mean±standard error. Significance is tested using the two-tailed Student's t-test (*p<. 05, ** p<. 01) and tested relative to old-GFP (n=4).

FIG. 11. Representative images of wound areas for old-GFP, old-SRSF1 (ON-ON), and young-GFP mice at day 11 post-wounding.

FIG. 12. Survivorship of wild-type (N2) and raga-1 (ok386) worms+/−rsp-3 RNAi (P<0.001) and (p-values comparing wildtype N2 on RNAi versus raga-1 (ok386) on RNAi, 3 replicates).

FIG. 13A-13B. FIG. 13A: % HSC in 2-week in vitro culture of CD34+ cells from a young or old donor after nucleofection with SRSF1 or GFP mRNA. There is an increase in the percentage of HSCs in the cells that received SRSF1 mRNA, suggesting higher self-renewal capacity in the stem cell population. FIG. 13B: % NK cells in 2-week in vitro culture of CD34+ cells from a young or old donor after nucleofection with SRSF1 or GFP mRNA. There is a higher percentage of NK cells in the SRSF1 treated cells, suggesting an increased lymphoid output.

### DETAILED DESCRIPTION

Aging is a complex process that manifests itself through a progressive multifaceted functional decline. Unstable transcriptomic profiles have emerged as a hallmark of aging organisms, and their modulation through interventions such as reprogramming has been shown to reverse aging signatures and improve function. Furthermore, aging clocks use gene expression information at the epigenetic or transcriptomic level to predict the biological age of a cell, but they currently lack the interpretability necessary for assessing cellular function and developing specific perturbations for cellular rejuvenation. To address this, here gene-cellular process associations were integrated with RNA-Sequencing datasets to develop a functionally interpretable transcriptomic age predictor. The RNA clock showed high predictive accuracy (r=0.93) when applied to multiple datasets, as well as robust response to known age-modulating interventions such as reprogramming and cell stressors. Moreover, the clock was used as an integrative aging assay and performed a transcriptomic reprogramming screen for rejuvenation of primary human fibroblasts. Using the predictor's functional interpretability, four different aging phenotypes were uncovered based on the process dysfunctions, which influenced the response to most of the perturbations. Nevertheless, SRSF1 overexpression led to a robust transcriptomic age reversal and functional improvement. These findings propose a new paradigm for aging as a transcriptomic state and SRSF1 as a promising target for cell rejuvenation.

### Serine and Arginine Rich Splicing Factor 1 (SRSF1)

Aspects of the present disclosure relate, at least in part, to methods and compositions for contacting a cell with a Serine and Arginine Splicing Factor 1 (SRSF1) protein or a nucleic acid encoding the SRSF1 protein to induce cellular rejuvenation and/or reverse or inhibit cellular senescence. As used herein, “SRSF1” refers to a full-length or truncated SRSF1 protein, a fragment of the SRSF1 protein, or a nucleic acid encoding the protein. Thus, an SRSF1 protein may be a wild-type, naturally occurring protein or it may be a variant of a wild-type SRSF1 protein (e.g., having at least 80%, at least 85%, at least 90%, at least 95%, at least 96%, at least 97%, at least 98%, or at least 99% identity to a wild-type SRSF1 protein). The experiments described herein resulted in the identification of SRSF1 as a gene involved in the reversal of transcriptomic age of cells and the reduction of cellular senescence of cells, such as fibroblasts, from middle-aged and elderly donors. The SRSF1 gene encodes a member of the arginine/serine-rich splicing factor protein family. The encoded SRSF1 protein can either activate or repress splicing, depending on its phosphorylation state and its interaction partners. Multiple transcript variants have been found for this gene; and there is a pseudogene of this gene on chromosome 13. SRSF1 plays a role in preventing exon skipping, ensuring the accuracy of splicing and regulating alternative splicing. The following SRSF1 sequences may be used in accordance with any of the embodiments provided herein.

A non-limiting example of a human SRSF1 nucleic acid coding sequence is provided by SEQ ID NO: 1:

(SEQ ID NO: 1)
ATGTCGGGAG GTGGTGTGAT TCGTGGCCCC GCAGGGAACA
ACGATTGCCG CATCTACGTG GGTAACTTAC CTCCAGACAT
CCGAACCAAG GACATTGAGG ACGTGTTCTA CAAATACGGC
GCTATCCGCG ACATCGACCT CAAGAATCGC CGCGGGGGAC
CGCCCTTCGC CTTCGTTGAG TTCGAGGACC CGCGAGACGC
GGAAGACGCG GTGTATGGTC GCGACGGCTA TGATTACGAT
GGGTACCGTC TGCGGGTGGA GTTTCCTCGA AGCGGCCGTG
GAACAGGCCG AGGCGGCGGC GGGGGTGGAG GTGGCGGAGC
TCCCCGAGGT CGCTATGGCC CCCCATCCAG GCGGTCTGAA
AACAGAGTGG TTGTCTCTGG ACTGCCTCCA AGTGGAAGTT
GGCAGGATTT AAAGGATCAC ATGCGTGAAG CAGGTGATGT
ATGTTATGCT GATGTTTACC GAGATGGCAC TGGTGTCGTG
GAGTTTGTAC GGAAAGAAGA TATGACCTAT GCAGTTCGAA
AACTGGATAA CACTAAGTTT AGATCTCATG AGGGAGAAAC
TGCCTACATC CGGGTTAAAG TTGATGGGCC CAGAAGTCCA
AGTTATGGAA GATCTCGATC TCGAAGCCGT AGTCGTAGCA
GAAGCCGTAG CAGAAGCAAC AGCAGGAGTC GCAGTTACTC
CCCAAGGAGA AGCAGAGGAT CACCACGCTA TTCTCCCCGT
CATAGCAGAT CTCGCTCTCG TACATAA

A non-limiting example of a human SRSF1 protein sequence is provided by SEQ ID NO: 2, which corresponds to the sequence provided by UniProtKB Accession No. Q07955:

(SEQ ID NO: 2)
MSGGGVIRGP AGNNDCRIYV GNLPPDIRTK DIEDVFYKYG
AIRDIDLKNR RGGPPFAFVE FEDPRDAEDA VYGRDGYDYD
GYRLRVEFPR SGRGTGRGGG GGGGGGAPRG RYGPPSRRSE
NRVVVSGLPP SGSWQDLKDH MREAGDVCYA DVYRDGTGVV
EFVRKEDMTY AVRKLDNTKF RSHEGETAYI RVKVDGPRSP
SYGRSRSRSR SRSRSRSRSN SRSRSYSPRR SRGSPRYSPR
HSRSRSRT

### Solute Carrier Family 2 Member 13 (SLC2A13)

Aspects of the present disclosure relate, at least in part, to methods and compositions for contacting a cell with a Solute Carrier Family 2 Member 13 (SLC2A13) protein or a nucleic acid encoding the SLC2A13 protein to induce cellular rejuvenation and/or reverse or inhibit cellular senescence. As used herein, “SLC2A13” refers to a full-length or truncated SLC2A13 protein, a fragment of the SLC2A13 protein, or a nucleic acid encoding the protein. Thus, an SLC2A13 protein may be a wild-type, naturally occurring protein or it may be a variant of a wild-type SLC2A13 protein (e.g., having at least 80%, at least 85%, at least 90%, at least 95%, at least 96%, at least 97%, at least 98%, or at least 99% identity to a wild-type SLC2A13 protein). The experiments described herein also resulted in the identification of SLC2A13 as a gene involved in the reversal of transcriptomic age of cells and the reduction of cellular senescence of cells, such as fibroblasts, from middle-aged and elderly donors. SLC2A13 is involved in myo-inositol transport and positive regulation of amyloid-beta formation. The following SLC2A13 sequences may be used in accordance with any of the embodiments provided herein.

A non-limiting example of a human SLC2A13 protein sequence is provided by SEQ ID NO: 5, which corresponds to the sequence provided by UniProtKB Accession No. Q96QE2:

(SEQ ID NO: 5)
MSRKASENVE YTLRSLSSLM GERRRKQPEP DAASAAGECS
LLAAAESSTS LQSAGAGGGG VGDLERAARR QFQQDETPAF
VYVVAVFSAL GGFLFGYDTG VVSGAMLLLK RQLSLDALWQ
ELLVSSTVGA AAVSALAGGA LNGVFGRRAA ILLASALFTA
GSAVLAAANN KETLLAGRLV VGLGIGIASM TVPVYIAEVS
PPNLRGRLVT INTLFITGGQ FFASVVDGAF SYLQKDGWRY
MLGLAAVPAV IQFFGFLFLP ESPRWLIQKG QTQKARRILS
QMRGNQTIDE EYDSIKNNIE EEEKEVGSAG PVICRMLSYP
PTRRALIVGC GLQMFQQLSG INTIMYYSAT ILQMSGVEDD
RLAIWLASVT AFTNFIFTLV GVWLVEKVGR RKLTFGSLAG
TTVALIILAL GFVLSAQVSP RITFKPIAPS GQNATCTRYS
YCNECMLDPD CGFCYKMNKS TVIDSSCVPV NKASTNEAAW
GRCENETKFK TEDIFWAYNF CPTPYSWTAL LGLILYLVFF
APGMGPMPWT VNSEIYPLWA RSTGNACSSG INWIFNVLVS
LTFLHTAEYL TYYGAFFLYA GFAAVGLLFI YGCLPETKGK
KLEEIESLFD NRLCTCGTSD SDEGRYIEYI RVKGSNYHLS
DNDASDVE

### 2-5A-Dependent Ribonuclease (RNASEL)

Aspects of the present disclosure relate, at least in part, to methods and compositions for contacting a cell with a 2-5A-dependent ribonuclease (RNASEL) protein or a nucleic acid encoding the RNASEL protein to induce cellular rejuvenation and/or reverse or inhibit cellular senescence. As used herein, “RNASEL” refers to a full-length or truncated RNASEL protein, a fragment of the RNASEL protein, or a nucleic acid encoding the protein. Thus, an RNASEL protein may be a wild-type, naturally occurring protein or it may be a variant of a wild-type RNASEL protein (e.g., having at least 80%, at least 85%, at least 90%, at least 95%, at least 96%, at least 97%, at least 98%, or at least 99% identity to a wild-type RNASEL protein). The experiments described herein also resulted in the identification of RNASEL as a gene involved in the reversal of transcriptomic age of cells and the reduction of cellular senescence of cells, such as fibroblasts, from middle-aged and elderly donors. RNASEL is an endoribonuclease that functions in the interferon (IFN) antiviral response. RNASEL mediated apoptosis is the result of a JNK-dependent stress-response pathway leading to cytochrome c release from mitochondria and caspase-dependent apoptosis. The following RNASEL sequences may be used in accordance with any of the embodiments provided herein.

A non-limiting example of a human RNASEL protein sequence is provided by SEQ ID NO: 6, which corresponds to the sequence provided by UniProtKB Accession No. Q05823:

(SEQ ID NO: 6)
MESRDHNNPQ EGPTSSSGRR AAVEDNHLLI KAVQNEDVDL
VQQLLEGGAN VNFQEEEGGW TPLHNAVQMS REDIVELLLR
HGADPVLRKK NGATPFILAA IAGSVKLLKL FLSKGADVNE
CDFYGFTAFM EAAVYGKVKA LKFLYKRGAN VNLRRKTKED
QERLRKGGAT ALMDAAEKGH VEVLKILLDE MGADVNACDN
MGRNALIHAL LSSDDSDVEA ITHLLLDHGA DVNVRGERGK
TPLILAVEKK HLGLVQRLLE QEHIEINDTD SDGKTALLLA
VELKLKKIAE LLCKRGASTD CGDLVMTARR NYDHSLVKVL
LSHGAKEDFH PPAEDWKPQS SHWGAALKDL HRIYRPMIGK
LKFFIDEKYK IADTSEGGIY LGFYEKQEVA VKTFCEGSPR
AQREVSCLQS SRENSHLVTF YGSESHRGHL FVCVTLCEQT
LEACLDVHRG EDVENEEDEF ARNVLSSIFK AVQELHLSCG
YTHQDLQPQN ILIDSKKAAH LADFDKSIKW AGDPQEVKRD
LEDLGRLVLY VVKKGSISFE DLKAQSNEEV VQLSPDEETK
DLIHRLFHPG EHVRDCLSDL LGHPFFWTWE SRYRTLRNVG
NESDIKTRKS ESEILRLLQP GPSEHSKSFD KWTTKINECV
MKKMNKFYEK RGNFYQNTVG DLLKFIRNLG EHIDEEKHKK
MKLKIGDPSL YFQKTFPDLV IYVYTKLQNT EYRKHFPQTH
SPNKPQCDGA GGASGLASPG C

### Wd and Tetratricopeptide Repeats Protein 1 (WdTC1)

Aspects of the present disclosure relate, at least in part, to methods and compositions for contacting a cell with a WD and tetratricopeptide repeats protein 1 (WDTC1) protein or a nucleic acid encoding the WDTC1 protein to induce cellular rejuvenation and/or reverse or inhibit cellular senescence. As used herein, “WDTC1” refers to a full-length or truncated WDTC1 protein, a fragment of the WDTC1 protein, or a nucleic acid encoding the protein. Thus, an WDTC1 protein may be a wild-type, naturally occurring protein or it may be a variant of a wild-type WDTC1 protein (e.g., having at least 80%, at least 85%, at least 90%, at least 95%, at least 96%, at least 97%, at least 98%, or at least 99% identity to a wild-type WDTC1 protein). The experiments described herein also resulted in the identification of WDTC1 as a gene involved in the reversal of transcriptomic age of cells and the reduction of cellular senescence of cells, such as fibroblasts, from middle-aged and elderly donors. WDTC1 is involved in the pathway protein ubiquitination and is predicted to enable enzyme inhibitor activity; histone binding activity; and histone deacetylase binding activity. The following WDTC1 sequences may be used in accordance with any of the embodiments provided herein.

A non-limiting example of a human WDTC1 protein sequence is provided by SEQ ID NO: 7, which corresponds to the sequence provided by UniProtKB Accession No. Q8N5D0-4:

(SEQ ID NO: 7)
MAKVNITRDL IRRQIKERGA LSFERRYHVT DPFIRRIGLE
AELQGHSGCV NCLEWNEKGD LLASGSDDQH TIVWDPLHHK
KLLSMHTGHT ANIFSVKFLP HAGDRILITG AADSKVHVHD
LTVKETIHMF GDHTNRVKRI ATAPMWPNTF WSAAEDGLIR
QYDLRENSKH SEVLIDLTEY CGQLVEAKCL TVNPQDNNCL
AVGASGPFVR LYDIRMIHNH RKSMKQSPSA GVHTFCDRQK
PLPDGAAQYY VAGHLPVKLP DYNNRLRVLV ATYVTFSPNG
TELLVNMGGE QVYLFDLTYK QRPYTFLLPR KCHSSGVQNG
KMSTNGVSNG VSNGLHLHSN GFRLPESRGH VSPQVELPPY
LERVKQQANE AFACQQWTQA IQLYSKAVQR APHNAMLYGN
RAAAYMKRKW DGDHYDALRD CLKAISLNPC HLKAHFRLAR
CLFELKYVAE ALECLDDFKG KFPEQAHSSA CDALGRDITA
ALFSKNDGEE KKGPGGGAPV RLRSTSRKDS ISEDEMVLRE
RSYDYQFRYC GHCNTTTDIK EANFFGSNAQ YIVSGSDDGS
FFIWEKETTN LVRVLQGDES IVNCLQPHPS YCFLATSGID
PVVRLWNPRP ESEDLTGRVV EDMEGASQAN QRRMNADPLE
VMLLNMGYRI TGLSSGGAGA SDDEDSSEGQ VQCRPS

### Nucleophosmin 1 (NPM1)

Aspects of the present disclosure relate, at least in part, to methods and compositions for contacting a cell with a Nucleophosmin 1 (NPM1) protein or a nucleic acid encoding the NPM1 protein to induce cellular rejuvenation and/or reverse or inhibit cellular senescence. As used herein, “NPM1” refers to a full-length or truncated NPM1 protein, a fragment of the NPM1 protein, or a nucleic acid encoding the protein. Thus, an NPM1 protein may be a wild-type, naturally occurring protein, or it may be a variant of a wild-type NPM1 protein (e.g., having at least 80%, at least 85%, at least 90%, at least 95%, at least 96%, at least 97%, at least 98%, or at least 99% identity to a wild-type NPM1 protein). The experiments described herein also resulted in the identification of NPM1 as a gene involved in the reversal of transcriptomic age of cells and the reduction of cellular senescence of cells, such as fibroblasts, from middle-aged and elderly donors. NPM1 is involved in the pathway protein ubiquitination and is predicted to enable enzyme inhibitor activity; histone binding activity; and histone deacetylase binding activity. The following NPM1 sequences may be used in accordance with any of the embodiments provided herein.

A non-limiting example of a human NPM1 protein sequence is provided by SEQ ID NO: 8, which corresponds to the sequence provided by UniProtKB Accession No. P06748:

(SEQ ID NO: 8)
MEDSMDMDMS PLRPQNYLFG CELKADKDYH FKVDNDENEH
QLSLRTVSLG AGAKDELHIV EAEAMNYEGS PIKVTLATLK
MSVQPTVSLG GFEITPPVVL RLKCGSGPVH ISGQHLVAVE
EDAESEDEEE EDVKLLSISG KRSAPGGGSK VPQKKVKLAA
DEDDDDDDEE DDDEDDDDDD FDDEEAEEKA PVKKSIRDTP
AKNAQKSNON GKDSKPSSTP RSKGQESFKK QEKTPKTPKG
PSSVEDIKAK MQASIEKGGS LPKVEAKFIN YVKNCFRMTD
QEAIQDLWQW RKSL

### Histone Acetyltransferase KAT7 (KAT7)

Aspects of the present disclosure relate, at least in part, to methods and compositions for contacting a cell with an inhibitor of a Histone acetyltransferase KAT7 (KAT7) protein, or an inhibitor of a nucleic acid encoding the KAT7 protein, to reduce KAT7 protein and/or nucleic acid expression levels in the cell to induce cellular rejuvenation and/or reverse or inhibit cellular senescence. The inhibitor may be a direct inhibitor or an indirect inhibitor of KAT7. In some embodiments, the inhibitor of KAT7 is a protein-based inhibitor, such as an antibody. In some embodiments, the inhibitor of KAT7 is a programmable gene editing system (e.g., including a Transcription activator-like effector nucleases (TALEN), a Zinc Finger Nuclease (ZFN), or a CRISPR-based system (e.g., a CRISPR-Cas9 system, a CRISPRi system, or a CRISPRoff system)). In some embodiments, the inhibitor is of KAT7 an RNA interference (RNAi) molecule (e.g., an shRNA, an siRNA, or an miRNA). In some embodiments, the inhibitor is a small molecule drug inhibitor (having a molecular weight of less than or equal to 1000 Da). As used herein, “KAT7” refers to a full-length or truncated KAT7 protein, a fragment of the KAT7 protein, or a nucleic acid encoding the protein. Thus, a KAT7 protein may be a wild-type, naturally occurring protein or it may be a variant of a wild-type KAT7 protein (e.g., having at least 80%, at least 85%, at least 90%, at least 95%, at least 96%, at least 97%, at least 98%, or at least 99% identity to a wild-type KAT7 protein). The experiments described herein also resulted in the identification of KAT7 as a gene involved in the reversal of transcriptomic age of cells and the reduction of cellular senescence of cells, such as fibroblasts, from middle-aged and elderly donors. KAT7 is involved in the pathway protein ubiquitination and is predicted to enable enzyme inhibitor activity; histone binding activity; and histone deacetylase binding activity. The following KAT7 sequences may be used in accordance with any of the embodiments provided herein.

A non-limiting example of a human KAT7 protein sequence is provided by SEQ ID NO: 9, which corresponds to the sequence provided by UniProtKB Accession No. 095251:

(SEQ ID NO: 9)
MPRRKRNAGS SSDGTEDSDF STDLEHTDSS ESDGTSRRSA
RVTRSSARLS QSSQDSSPVR NLQSFGTEEP AYSTRRVTRS
QQQPTPVTPK KYPLRQTRSS GSETEQVVDF SDRETKNTAD
HDESPPRTPT GNAPSSESDI DISSPNVSHD ESIAKDMSLK
DSGSDLSHRP KRRRFHESYN FNMKCPTPGC NSLGHLTGKH
ERHFSISGCP LYHNLSADEC KVRAQSRDKQ IEERMLSHRQ
DDNNRHATRH QAPTERQLRY KEKVAELRKK RNSGLSKEQK
EKYMEHRQTY GNTREPLLEN LTSEYDLDLF RRAQARASED
LEKLRLQGQI TEGSNMIKTI AFGRYELDTW YHSPYPEEYA
RLGRLYMCEF CLKYMKSQTI LRRHMAKCVW KHPPGDEIYR
KGSISVFEVD GKKNKIYCQN LCLLAKLFLD HKTLYYDVEP
FLFYVMTEAD NTGCHLIGYF SKEKNSFLNY NVSCILTMPQ
YMRQGYGKML IDFSYLLSKV EEKVGSPERP LSDLGLISYR
SYWKEVLLRY LHNFQGKEIS IKEISQETAV NPVDIVSTLQ
ALQMLKYWKG KHLVLKRQDL IDEWIAKEAK RSNSNKTMDP
SCLKWTPPKG T

### Estrogen Receptor (ESR1)

Aspects of the present disclosure relate, at least in part, to methods and compositions for contacting a cell with an inhibitor of a Estrogen receptor (ESR1) protein or a nucleic acid encoding the ESR1 protein to reduce ESR1 protein and/or nucleic acid expression levels in the cell to induce cellular rejuvenation and/or reverse or inhibit cellular senescence. The inhibitor may be a direct inhibitor or an indirect inhibitor of ESR1. In some embodiments, the inhibitor of ESR1 is a protein-based inhibitor, such as an antibody. In some embodiments, the inhibitor of ESR1 is a programmable gene editing system (e.g., including a Transcription activator-like effector nucleases (TALEN), a Zinc Finger Nuclease (ZFN), or a CRISPR-based system (e.g., a CRISPR-Cas9 system, a CRISPRi system, or a CRISPRoff system)). In some embodiments, the inhibitor is of ESR1 an RNA interference (RNAi) molecule (e.g., an shRNA, an siRNA, or an miRNA). In some embodiments, the inhibitor is a small molecule drug inhibitor (having a molecular weight of less than or equal to 1000 Da). As used herein, “ESR1” refers to a full-length or truncated ESR1 protein, a fragment of the ESR1 protein, or a nucleic acid encoding the protein. Thus, a ESR1 protein may be a wild-type, naturally occurring protein or it may be a variant of a wild-type ESR1 protein (e.g., having at least 80%, at least 85%, at least 90%, at least 95%, at least 96%, at least 97%, at least 98%, or at least 99% identity to a wild-type ESR1 protein). The experiments described herein also resulted in the identification of ESR1 as a gene involved in the reversal of transcriptomic age of cells and the reduction of cellular senescence of cells, such as fibroblasts, from middle-aged and elderly donors. ESR1 is involved in the pathway protein ubiquitination and is predicted to enable enzyme inhibitor activity; histone binding activity; and histone deacetylase binding activity. The following ESR1 sequences may be used in accordance with any of the embodiments provided herein.

A non-limiting example of a human ESR1 protein sequence is provided by SEQ ID NO: 10, which corresponds to the sequence provided by UniProtKB Accession No. P03372:

(SEQ ID NO: 10)
MTMTLHTKAS GMALLHQIQG NELEPLNRPQ LKIPLERPLG
EVYLDSSKPA VYNYPEGAAY EFNAAAAANA QVYGQTGLPY
GPGSEAAAFG SNGLGGFPPL NSVSPSPLML LHPPPQLSPF
LQPHGQQVPY YLENEPSGYT VREAGPPAFY RPNSDNRRQG
GRERLASTND KGSMAMESAK ETRYCAVCND YASGYHYGVW
SCEGCKAFFK RSIQGHNDYM CPATNQCTID KNRRKSCQAC
RLRKCYEVGM MKGGIRKDRR GGRMLKHKRQ RDDGEGRGEV
GSAGDMRAAN LWPSPLMIKR SKKNSLALSL TADQMVSALL
DAEPPILYSE YDPTRPFSEA SMMGLLTNLA DRELVHMINW
AKRVPGFVDL TLHDQVHLLE CAWLEILMIG LVWRSMEHPG
KLLFAPNLLL DRNQGKCVEG MVEIFDMLLA TSSRFRMMNL
QGEEFVCLKS IILLNSGVYT FLSSTLKSLE EKDHIHRVLD
KITDTLIHLM AKAGLTLQQQ HQRLAQLLLI LSHIRHMSNK
GMEHLYSMKC KNVVPLYDLL LEMLDAHRLH APTSRGGASV
EETDQSHLAT AGSTSSHSLQ KYYITGEAEG FPATV

### Mitogen-Activated Protein Kinase 7 (MAPK7)

Aspects of the present disclosure relate, at least in part, to methods and compositions for contacting a cell with an inhibitor of a Mitogen-activated protein kinase 7 (MAPK7) protein or a nucleic acid encoding the MAPK7 protein to reduce MAPK7 protein and/or nucleic acid expression levels in the cell to induce cellular rejuvenation and/or reverse or inhibit cellular senescence. The inhibitor may be a direct inhibitor or an indirect inhibitor of MAPK7. In some embodiments, the inhibitor of MAPK7 is a protein-based inhibitor, such as an antibody. In some embodiments, the inhibitor of MAPK7 is a programmable gene editing system (e.g., including a Transcription activator-like effector nucleases (TALEN), a Zinc Finger Nuclease (ZFN), or a CRISPR-based system (e.g., a CRISPR-Cas9 system, a CRISPRi system, or a CRISPRoff system)). In some embodiments, the inhibitor is of MAPK7 an RNA interference (RNAi) molecule (e.g., an shRNA, an siRNA, or an miRNA). In some embodiments, the inhibitor is a small molecule drug inhibitor (having a molecular weight of less than or equal to 1000 Da). As used herein, “MAPK7” refers to a full-length or truncated MAPK7 protein, a fragment of the MAPK7 protein, or a nucleic acid encoding the protein. Thus, a MAPK7 protein may be a wild-type, naturally occurring protein or it may be a variant of a wild-type MAPK7 protein (e.g., having at least 80%, at least 85%, at least 90%, at least 95%, at least 96%, at least 97%, at least 98%, or at least 99% identity to a wild-type MAPK7 protein). The experiments described herein also resulted in the identification of MAPK7 as a gene involved in the reversal of transcriptomic age of cells and the reduction of cellular senescence of cells, such as fibroblasts, from middle-aged and elderly donors. MAPK7 is involved in the pathway protein ubiquitination and is predicted to enable enzyme inhibitor activity; histone binding activity; and histone deacetylase binding activity. The following MAPK7 sequences may be used in accordance with any of the embodiments provided herein.

A non-limiting example of a human MAPK7 protein sequence is provided by SEQ ID NO: 11, which corresponds to the sequence provided by UniProtKB Accession No. Q13164:

(SEQ ID NO: 11)
MAEPLKEEDG EDGSAEPPGP VKAEPAHTAA SVAAKNLALL
KARSFDVTFD VGDEYEIIET IGNGAYGVVS SARRRLTGQQ
VAIKKIPNAF DVVTNAKRTL RELKILKHFK HDNIIAIKDI
LRPTVPYGEF KSVYVVLDLM ESDLHQIIHS SQPLTLEHVR
YFLYQLLRGL KYMHSAQVIH RDLKPSNLLV NENCELKIGD
FGMARGLCTS PAEHQYFMTE YVATRWYRAP ELMLSLHEYT
QAIDLWSVGC IFGEMLARRQ LFPGKNYVHQ LQLIMMVLGT
PSPAVIQAVG AERVRAYIQS LPPRQPVPWE TVYPGADRQA
LSLLGRMLRF EPSARISAAA ALRHPFLAKY HDPDDEPDCA
PPFDFAFDRE ALTRERIKEA IVAEIEDFHA RREGIRQQIR
FQPSLQPVAS EPGCPDVEMP SPWAPSGDCA MESPPPAPPP
CPGPAPDTID LTLQPPPPVS EPAPPKKDGA ISDNTKAALK
AALLKSLRSR LRDGPSAPLE APEPRKPVTA QERQREREEK
RRRRQERAKE REKRRQERER KERGAGASGG PSTDPLAGLV
LSDNDRSLLE RWTRMARPAA PALTSVPAPA PAPTPTPTPV
QPTSPPPGPV AQPTGPQPQS AGSTSGPVPQ PACPPPGPAP
HPTGPPGPIP VPAPPQIATS TSLLAAQSLV PPPGLPGSST
PGVLPYFPPG LPPPDAGGAP QSSMSESPDV NLVTQQLSKS
QVEDPLPPVF SGTPKGSGAG YGVGFDLEEF LNQSFDMGVA
DGPQDGQADS ASLSASLLAD WLEGHGMNPA DIESLQREIQ
MDSPMLLADL PDLQDP

### Lysine-Specific Demethylase 6A (KDM6A)

Aspects of the present disclosure relate, at least in part, to methods and compositions for contacting a cell with an inhibitor of a Lysine-specific demethylase 6A (KDM6A) protein or a nucleic acid encoding the KDM6A protein to reduce KDM6A protein and/or nucleic acid expression levels in the cell to induce cellular rejuvenation and/or reverse or inhibit cellular senescence. The inhibitor may be a direct inhibitor or an indirect inhibitor of KDM6A. In some embodiments, the inhibitor of KDM6A is a protein-based inhibitor, such as an antibody. In some embodiments, the inhibitor of KDM6A is a programmable gene editing system (e.g., including a Transcription activator-like effector nucleases (TALEN), a Zinc Finger Nuclease (ZFN), or a CRISPR-based system (e.g., a CRISPR-Cas9 system, a CRISPRi system, or a CRISPRoff system)). In some embodiments, the inhibitor is of KDM6A an RNA interference (RNAi) molecule (e.g., an shRNA, an siRNA, or an miRNA). In some embodiments, the inhibitor is a small molecule drug inhibitor (having a molecular weight of less than or equal to 1000 Da). As used herein, “KDM6A” refers to a full-length or truncated KDM6A protein, a fragment of the KDM6A protein, or a nucleic acid encoding the protein. Thus, a KDM6A protein may be a wild-type, naturally occurring protein or it may be a variant of a wild-type KDM6A protein (e.g., having at least 80%, at least 85%, at least 90%, at least 95%, at least 96%, at least 97%, at least 98%, or at least 99% identity to a wild-type KDM6A protein). The experiments described herein also resulted in the identification of KDM6A as a gene involved in the reversal of transcriptomic age of cells and the reduction of cellular senescence of cells, such as fibroblasts, from middle-aged and elderly donors. KDM6A is involved in the pathway protein ubiquitination and is predicted to enable enzyme inhibitor activity; histone binding activity; and histone deacetylase binding activity. The following KDM6A sequences may be used in accordance with any of the embodiments provided herein.

A non-limiting example of a human KDM6A protein sequence is provided by SEQ ID NO: 12, which corresponds to the sequence provided by UniProtKB Accession No. 015550:

(SEQ ID NO: 12)
MKSCGVSLAT AAAAAAAFGD EEKKMAAGKA SGESEEASPS
LTAEEREALG GLDSRLFGFV RFHEDGARTK ALLGKAVRCY
ESLILKAEGK VESDFFCQLG HFNLLLEDYP KALSAYQRYY
SLQSDYWKNA AFLYGLGLVY FHYNAFQWAI KAFQEVLYVD
PSFCRAKEIH LRLGLMFKVN TDYESSLKHF QLALVDCNPC
TLSNAEIQFH IAHLYETQRK YHSAKEAYEQ LLQTENLSAQ
VKATVLQQLG WMHHTVDLLG DKATKESYAI QYLQKSLEAD
PNSGQSWYFL GRCYSSIGKV QDAFISYRQS IDKSEASADT
WCSIGVLYQQ QNQPMDALQA YICAVQLDHG HAAAWMDLGT
LYESCNQPQD AIKCYLNATR SKSCSNTSAL AARIKYLQAQ
LCNLPQGSLQ NKTKLLPSIE EAWSLPIPAE LTSRQGAMNT
AQQNTSDNWS GGHAVSHPPV QQQAHSWCLT PQKLQHLEQL
RANRNNLNPA QKLMLEQLES QFVLMQQHQM RPTGVAQVRS
TGIPNGPTAD SSLPTNSVSG QQPQLALTRV PSVSQPGVRP
ACPGQPLANG PFSAGHVPCS TSRTLGSTDT ILIGNNHITG
SGSNGNVPYL QRNALTLPHN RTNLTSSAEE PWKNQLSNST
QGLHKGQSSH SAGPNGERPL SSTGPSQHLQ AAGSGIQNQN
GHPTLPSNSV TQGAALNHLS SHTATSGGQQ GITLTKESKP
SGNILTVPET SRHTGETPNS TASVEGLPNH VHQMTADAVC
SPSHGDSKSP GLLSSDNPQL SALLMGKANN NVGTGTCDKV
NNIHPAVHTK TDNSVASSPS SAISTATPSP KSTEQTTTNS
VTSLNSPHSG LHTINGEGME ESQSPMKTDL LLVNHKPSPQ
IIPSMSVSIY PSSAEVLKAC RNLGKNGLSN SSILLDKCPP
PRPPSSPYPP LPKDKLNPPT PSIYLENKRD AFFPPLHQFC
TNPNNPVTVI RGLAGALKLD LGLFSTKTLV EANNEHMVEV
RTQLLQPADE NWDPTGTKKI WHCESNRSHT TIAKYAQYQA
SSFQESLREE NEKRSHHKDH SDSESTSSDN SGRRRKGPFK
TIKFGTNIDL SDDKKWKLQL HELTKLPAFV RVVSAGNLLS
HVGHTILGMN TVQLYMKVPG SRTPGHQENN NFCSVNINIG
PGDCEWFVVP EGYWGVLNDF CEKNNLNFLM GSWWPNLEDL
YEANVPVYRF IQRPGDLVWI NAGTVHWVQA IGWCNNIAWN
VGPLTACQYK LAVERYEWNK LQSVKSIVPM VHLSWNMARN
IKVSDPKLFE MIKYCLLRTL KQCQTLREAL IAAGKEIIWH
GRTKEEPAHY CSICEVEVFD LLFVTNESNS RKTYIVHCQD
CARKTSGNLE NFVVLEQYKM EDLMQVYDQF TLAPPLPSAS
S

### Catenin Beta-1 (CTNNB1)

Aspects of the present disclosure relate, at least in part, to methods and compositions for contacting a cell with an inhibitor of a Catenin beta-1 (CTNNB1) protein or a nucleic acid encoding the CTNNB1 protein to reduce CTNNB1 protein and/or nucleic acid expression levels in the cell to induce cellular rejuvenation and/or reverse or inhibit cellular senescence. The inhibitor may be a direct inhibitor or an indirect inhibitor of CTNNB1. In some embodiments, the inhibitor of CTNNB1 is a protein-based inhibitor, such as an antibody. In some embodiments, the inhibitor of CTNNB1 is a programmable gene editing system (e.g., including a Transcription activator-like effector nucleases (TALEN), a Zinc Finger Nuclease (ZFN), or a CRISPR-based system (e.g., a CRISPR-Cas9 system, a CRISPRi system, or a CRISPRoff system)). In some embodiments, the inhibitor is of CTNNB1 an RNA interference (RNAi) molecule (e.g., an shRNA, an siRNA, or an miRNA). In some embodiments, the inhibitor is a small molecule drug inhibitor (having a molecular weight of less than or equal to 1000 Da). As used herein, “CTNNB1” refers to a full-length or truncated CTNNB1 protein, a fragment of the CTNNB1 protein, or a nucleic acid encoding the protein. Thus, a CTNNB1 protein may be a wild-type, naturally occurring protein or it may be a variant of a wild-type CTNNB1 protein (e.g., having at least 80%, at least 85%, at least 90%, at least 95%, at least 96%, at least 97%, at least 98%, or at least 99% identity to a wild-type CTNNB1 protein). The experiments described herein also resulted in the identification of CTNNB1 as a gene involved in the reversal of transcriptomic age of cells and the reduction of cellular senescence of cells, such as fibroblasts, from middle-aged and elderly donors. CTNNB1 is involved in the pathway protein ubiquitination and is predicted to enable enzyme inhibitor activity; histone binding activity; and histone deacetylase binding activity. The following CTNNB1 sequences may be used in accordance with any of the embodiments provided herein.

A non-limiting example of a human CTNNB1 protein sequence is provided by SEQ ID NO: 13, which corresponds to the sequence provided by UniProtKB Accession No. P35222:

(SEQ ID NO: 13)
MATQADLMEL DMAMEPDRKA AVSHWQQQSY LDSGIHSGAT
TTAPSLSGKG NPEEEDVDTS QVLYEWEQGF SQSFTQEQVA
DIDGQYAMTR AQRVRAAMFP ETLDEGMQIP STQFDAAHPT
NVQRLAEPSQ MLKHAVVNLI NYQDDAELAT RAIPELTKLL
NDEDQVVVNK AAVMVHQLSK KEASRHAIMR SPQMVSAIVR
TMQNTNDVET ARCTAGTLHN LSHHREGLLA IFKSGGIPAL
VKMLGSPVDS VLFYAITTLH NLLLHQEGAK MAVRLAGGLQ
KMVALLNKTN VKFLAITTDC LQILAYGNQE SKLIILASGG
PQALVNIMRT YTYEKLLWTT SRVLKVLSVC SSNKPAIVEA
GGMQALGLHL TDPSQRLVQN CLWTLRNLSD AATKQEGMEG
LLGTLVQLLG SDDINVVTCA AGILSNLTCN NYKNKMMVCQ
VGGIEALVRT VLRAGDREDI TEPAICALRH LTSRHQEAEM
AQNAVRLHYG LPVVVKLLHP PSHWPLIKAT VGLIRNLALC
PANHAPLREQ GAIPRLVQLL VRAHQDTQRR TSMGGTQQQF
VEGVRMEEIV EGCTGALHIL ARDVHNRIVI RGLNTIPLFV
QLLYSPIENI QRVAAGVLCE LAQDKEAAEA IEAEGATAPL
TELLHSRNEG VATYAAAVLF RMSEDKPQDY KKRLSVELTS
SLFRTEPMAW NETADLGLDI GAQGEPLGYR QDDPSYRSFH
SGGYGQDALG MDPMMEHEMG GHHPGADYPV DGLPDLGHAQ
DLMDGLPPGD SNQLAWFDTD L

### Proteins

Aspects of the present disclosure relate, at least in part, to proteins that have been found to induce cellular rejuvenation and/or reverse or inhibit cellular senescence. The term “protein,” as used herein refers to a primary amino acid structure, a secondary amino acid structure, a natively unfolded amino acid structure, a folded tertiary amino acid structure, or a folded quaternary amino acid structure. In some embodiments, the protein is synthesized chemically. In some embodiments, the protein is translated from a nucleic acid structure. In some embodiments, the amino acid structure of the protein is mutated to include substitutions or deletions of specific amino acid residues.

Aspects of the present disclosure relate, at least in part, to proteins that have been found to induce aging, reduce cellular rejuvenation and/or accelerate cellular senescence. In some embodiments, inhibiting expression of said proteins induces cellular rejuvenation and/or reverses or inhibits cellular senescence. In some embodiments, said proteins are inhibited by a protein-based inhibitor, such as an antibody. In some embodiments, said proteins are inhibited by a programmable gene editing system (e.g., including a Transcription activator-like effector nucleases (TALEN), a Zinc Finger Nuclease (ZFN), or a CRISPR-based system (e.g., a CRISPR-Cas9 system, a CRISPRi system, or a CRISPRoff system)). In some embodiments, said proteins are inhibited by an RNA interference (RNAi) molecule (e.g., an shRNA, an siRNA, or an miRNA). In some embodiments, said proteins are inhibited by a small molecule drug inhibitor (having a molecular weight of less than or equal to 1000 Da).

The cells of the present disclosure, in some embodiments, comprise proteins encoded by engineered nucleic acids. The term “protein” encompasses full length functional SRSF1 proteins as well as full-length or truncated functional variants of a protein, unless stated otherwise. Thus, the term “protein” encompasses full length functional SRSF1, SLC2A13, RNASEL, WDTC1, NPM1, KAT7, ESR1, MAPK7, KDM6A, and CTNNB1 proteins as well as full-length or truncated functional variants of the SRSF1, SLC2A13, RNASEL, WDTC1, NPM1, KAT7, ESR1, MAPK7, KDM6A, and CTNNB1 protein, unless stated otherwise. Thus, in some embodiments, an SRSF1 protein comprises the sequence of SEQ ID NO: 2 or is encoded by a nucleic acid comprising a protein coding sequence of SEQ ID NO: 1. In other embodiments, an SRSF1 protein comprises a sequence having at least 70% identity to the sequence of SEQ ID NO: 2 or is encoded by a nucleic acid comprising a sequence having at least 70% identity to a protein coding sequence of SEQ ID NO: 1. In some embodiments, an SLC2A13 protein comprises the sequence of SEQ ID NO: 5. In some embodiments, an RNASEL protein comprises the sequence of SEQ ID NO: 6. In some embodiments, an WDTC1 protein comprises the sequence of SEQ ID NO: 7. In some embodiments, an NPM1 protein comprises the sequence of SEQ ID NO: 8. In some embodiments, a KAT7 protein comprises the sequence of SEQ ID NO: 9. In some embodiments, an ESR1 protein comprises the sequence of SEQ ID NO: 10. In some embodiments, a MAPK7 protein comprises the sequence of SEQ ID NO: 11. In some embodiments, a KDM6A protein comprises the sequence of SEQ ID NO: 12. In some embodiments, a CTNNB1 protein comprises the sequence of SEQ ID NO: 13.

In some embodiments, the amino acid structure of a protein described herein has at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identity to the amino acid sequence of any one of SEQ ID NOs: 2, 5, 6, 7, 8, 9, 10, 11, 12, or 13 (e.g., determined by global alignment).

### Functional Variants

The term “identity” or “sequence identity” (used interchangeable herein) refers to a relationship between the amino acid sequences of two or more peptides or polypeptides, as determined by comparing the sequences. Identity also refers to the degree of sequence relatedness between or among sequences as determined by the number of matches between strings of two or more amino acid residues. Identity measures the percent of identical matches between the smaller of two or more sequences with gap alignments (if any) addressed by a particular mathematical model or computer program. Identity of related peptides can be readily calculated by known methods. “Percent (%) identity” as it applies to peptide sequences is defined as the percentage of amino acid residues of a first sequence that is identical with the amino acid residues of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. In some embodiments, computer programs including Clustal Omega (Sievers et al., Mol Syst Biol. 2011 Oct. 11; 7:539) may be used for sequence alignment. In some embodiments, computer programs including BLAST®, NBLAST®, XBLAST® or Gapped BLAST® may be used for sequence alignment. In some embodiments, percent identify is determined by aligning a sequence to a reference sequence.

In some embodiments, an SRSF1 protein comprises a sequence having at least 70% identity to the sequence of SEQ ID NO: 2 and maintains the functions described herein (e.g., capable of inducing cellular rejuvenation of a cell, for example, reducing the senescence-associated β-galactosidase activity, and/or reducing the proteasomal activity of a cell). For example, a functional SRSF1 protein may comprise a sequence having at least 75%, at least 80%, at least 85%, at least 90%, at least 95%, at least 96%, at least 97%, at least 98%, or at least 99% identity to the sequence of SEQ ID NO: 2.

In some embodiments, a functional SRSF1 protein is encoded by a nucleic acid comprising a sequence having at least 70% identity to a protein coding sequence of SEQ ID NO: 1. For example, a functional SRSF1 protein may be encoded by a nucleic acid comprising a sequence having at least 75%, at least 80%, at least 85%, at least 90%, at least 95%, at least 96%, at least 97%, at least 98%, or at least 99% identity to a protein coding sequence of SEQ ID NO: 1.

In some embodiments, an SLC2A13 protein comprises a sequence having at least 70% identity to the sequence of SEQ ID NO: 5 and maintains the functions described herein (e.g., capable of inducing cellular rejuvenation of a cell, for example, reducing the senescence-associated β-galactosidase activity, and/or reducing the proteasomal activity of a cell). For example, a functional SLC2A13 protein may comprise a sequence having at least 75%, at least 80%, at least 85%, at least 90%, at least 95%, at least 96%, at least 97%, at least 98%, or at least 99% identity to the sequence of SEQ ID NO: 5.

In some embodiments, an RNASEL protein comprises a sequence having at least 70% identity to the sequence of SEQ ID NO: 6 and maintains the functions described herein (e.g., capable of inducing cellular rejuvenation of a cell, for example, reducing the senescence-associated β-galactosidase activity, and/or reducing the proteasomal activity of a cell). For example, a functional RNASEL protein may comprise a sequence having at least 75%, at least 80%, at least 85%, at least 90%, at least 95%, at least 96%, at least 97%, at least 98%, or at least 99% identity to the sequence of SEQ ID NO: 6.

In some embodiments, an WDTC1 protein comprises a sequence having at least 70% identity to the sequence of SEQ ID NO: 7 and maintains the functions described herein (e.g., capable of inducing cellular rejuvenation of a cell, for example, reducing the senescence-associated β-galactosidase activity, and/or reducing the proteasomal activity of a cell). For example, a functional WDTC1 protein may comprise a sequence having at least 75%, at least 80%, at least 85%, at least 90%, at least 95%, at least 96%, at least 97%, at least 98%, or at least 99% identity to the sequence of SEQ ID NO: 7.

In some embodiments, an NPM1 protein comprises a sequence having at least 70% identity to the sequence of SEQ ID NO: 8 and maintains the functions described herein (e.g., capable of inducing cellular rejuvenation of a cell, for example, reducing the senescence-associated β-galactosidase activity, and/or reducing the proteasomal activity of a cell). For example, a functional NPM1 protein may comprise a sequence having at least 75%, at least 80%, at least 85%, at least 90%, at least 95%, at least 96%, at least 97%, at least 98%, or at least 99% identity to the sequence of SEQ ID NO: 8.

### Nucleic Acids

Aspects of the present disclosure relate, at least in part, to nucleic acids that encode the proteins described herein. In some embodiments, the nucleic acids are engineered. In some embodiments, the nucleic acids are modified to increase expression levels. In some embodiments, the nucleic acids are mutated. In some embodiments, the nucleic acids are modified by a substitution, insertion, or deletion mutation. In some embodiments, the nucleic acids are modified by truncation. In some embodiments, a truncated nucleic acid encodes a functional protein. In some embodiments, the nucleic acids are fused to a signal sequence. In some embodiments, the nucleic acids are operably linked to a promoter.

The cells of the present disclosure, in some embodiments, comprise engineered nucleic acids. For example, an engineered nucleic acid may encode a protein having at least 70%, at least 75%, at least 80%, at least 85%, at least 90%, at least 95%, at least 96%, at least 97%, at least 98%, at least 99%, or 100% identity to a functional SRSF1 protein comprising the sequence of SEQ ID NO: 2. In some embodiments, an engineered nucleic acid encodes a protein having at least 70%, at least 75%, at least 80%, at least 85%, at least 90%, at least 95%, at least 96%, at least 97%, at least 98%, at least 99%, or 100% identity to a functional SLC2A13 protein comprising the sequence of SEQ ID NO: 5. In some embodiments, an engineered nucleic acid encodes a protein having at least 70%, at least 75%, at least 80%, at least 85%, at least 90%, at least 95%, at least 96%, at least 97%, at least 98%, at least 99%, or 100% identity to a functional RNASEL protein comprising the sequence of SEQ ID NO: 6. In some embodiments, an engineered nucleic acid encodes a protein having at least 70%, at least 75%, at least 80%, at least 85%, at least 90%, at least 95%, at least 96%, at least 97%, at least 98%, at least 99%, or 100% identity to a functional WDTC1 protein comprising the sequence of SEQ ID NO: 7. In some embodiments, an engineered nucleic acid encodes a protein having at least 70%, at least 75%, at least 80%, at least 85%, at least 90%, at least 95%, at least 96%, at least 97%, at least 98%, at least 99%, or 100% identity to a functional NPM1 protein comprising the sequence of SEQ ID NO: 8.

An engineered nucleic acid is a polynucleotide (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. The MegaGate molecular cloning method may also be used. MegaGate is a toxin-less Gateway technology that eliminates the ccdb toxin used in Gateway recombinase cloning and instead utilizes meganuclease-mediated digestion to eliminate background vectors during cloning (see, e.g., Kramme C. et al. STAR Protoc. 2021 Oct. 22; 2 (4): 100907, incorporated herein by reference). Other methods of producing engineered polynucleotides may be used in accordance with the present disclosure.

In some embodiments, an engineered nucleic acid comprises a promoter operably linked to an open reading frame. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked.

In some embodiments, a promoter is an inducible promoter. An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). In some embodiments, the inducible promoter is a tetracycline-inducible promoter. In some embodiments, the inducible promoter is a doxycycline-inducible promoter. In other embodiments, a promoter is a constitutive promoter (active in vivo, unregulated).

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

Vectors used for delivery of an engineered nucleic acids include viral vectors and non-viral vectors. Non-limiting examples of viral vectors include retrovirus, adenovirus, adeno-associated virus (AAV), and herpes simplex virus. Non-limiting examples of non-viral vectors include minicircles, plasmids, bacterial artificial chromosomes (BACs), and yeast artificial chromosomes. Transposon-based systems, such as the piggyBac™ system (e.g., Chen et al. Nature Communications. 2020; 11 (1): 3446), may be used as a vector system to deliver an engineered nucleic acid. Other non-limiting examples include nanoparticle-based systems, such as lipid nanoparticles.

### Cellular Rejuvenation

Some aspects provide methods of inducing cellular rejuvenation of a cell, for example, to counteract the effects of aging. Aging in mammals has been summarized and categorized into nine “hallmarks” of aging: genomic instability, telomere attrition, epigenetic alterations, loss of proteostasis, deregulated nutrient sensing, mitochondrial dysfunction, cellular senescence, stem cell exhaustion, and altered intracellular communication (López-Otín C et al. Cell 2013; 153:1194-1217). Cellular rejuvenation is a process that not only delays aging but reverts it, leading to a younger cell. Cellular rejuvenation can decrease or eliminate age-accumulated damage and aging hallmarks collected during the life of a cell. Thus, “inducing cellular rejuvenation” refers to a process (method) that initiates the reversal of a hallmark of aging. Induction of cellular rejuvenation may be assessed, for example, by assessing the transcriptomic profile, gene expression of one or more nuclear and/or epigenetic markers, proteolytic activity, mitochondria health and/or function, expression of one or more SASP cytokines, or the methylation landscape of the cell, compared to a control (e.g., a reference value, for example, obtained from a young cell or an aged cell).

Aspects of the present disclosure relate to a method of inducing cellular rejuvenation of a cell comprising contacting the cell with an effective amount of a Serine and Arginine Rich Splicing Factor 1 (SRSF1) protein or a nucleic acid encoding the SRSF1 protein, a Solute Carrier Family Member 13 (SLC2A13) protein or a nucleic acid encoding the SLC2A13 protein, a 2-5A-dependent ribonuclease (RNASEL) protein or a nucleic acid encoding the RNASEL protein, a WD and tetratricopeptide repeats protein 1 (WDTC1) protein or a nucleic acid encoding the WDTC1 protein, or an NPM1 protein or a nucleic acid encoding the NPM1 protein. In some embodiments, the present disclosure relates to a method of inducing cellular rejuvenation of a cell comprising contacting the cell with an effective amount of a Serine and Arginine Rich Splicing Factor 1 (SRSF1) protein or a nucleic acid encoding the SRSF1 protein.

Aspects of the present disclosure relate to a method of inducing cellular rejuvenation of a cell in a subject comprising administering to the subject an effective amount of a Serine and Arginine Rich Splicing Factor 1 (SRSF1) protein or a nucleic acid encoding the SRSF1 protein, a Solute Carrier Family Member 13 (SLC2A13) protein or a nucleic acid encoding the SLC2A13 protein, a 2-5A-dependent ribonuclease (RNASEL) protein or a nucleic acid encoding the RNASEL protein, a WD and tetratricopeptide repeats protein 1 (WDTC1) protein or a nucleic acid encoding the WDTC1 protein, or an NPM1 protein or a nucleic acid encoding the NPM1 protein. In some embodiments, the present disclosure relates to a method of inducing cellular rejuvenation of a cell in a subject comprising administering to the subject an effective amount of a SRSF1 protein or a nucleic acid encoding the SRSF1 protein.

Aspects of the present disclosure relate to a method of inducing cellular rejuvenation of a cell comprising contacting the cell with an effective amount of an inhibitor of KAT7 protein expression or expression of a nucleic acid encoding the KAT7 protein, ESR1 protein expression or expression of a nucleic acid encoding the ESR1 protein, MAPK7 protein expression or expression of a nucleic acid encoding the MAPK7 protein, KDM6A protein expression or expression of a nucleic acid encoding the KDM6A protein, or CTNNB1 protein expression or expression of a nucleic acid encoding the CTNNB1 protein.

Aspects of the present disclosure relate to a method of inducing cellular rejuvenation of a cell in a subject comprising administering to the subject an effective amount of an inhibitor of KAT7 protein expression or expression of a nucleic acid encoding the KAT7 protein, ESR1 protein expression or expression of a nucleic acid encoding the ESR1 protein, MAPK7 protein expression or expression of a nucleic acid encoding the MAPK7 protein, KDM6A protein expression or expression of a nucleic acid encoding the KDM6A protein, or CTNNB1 protein expression or expression of a nucleic acid encoding the CTNNB1 protein.

Aspects of the present disclosure relate to a method of inducing cellular rejuvenation of a cell in a subject comprising administering to the subject an effective amount of one or more anti-aging proteins and an effective amount of an inhibitor of one or more pro-aging proteins. In some embodiments, the one or more anti-aging genes is selected from the group consisting of: SRSF1, SLC2A13, RNASEL, WDTC1, and NPM1. In some embodiments, the one or more pro-aging proteins is selected from the group consisting of: KAT7, ESR1, MAPK7, KDM6A, and CTNNB1.

In some embodiments, the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years. In some embodiments, the effective amount is sufficient to induce an average cellular rejuvenation of at least 25 years.

Aspects of the present disclosure relate to a method of inducing cellular rejuvenation of a cell comprising overexpressing in the cell an effective amount of a SRSF1 protein. In some embodiments, the method comprises activating expression or activity of endogenous SRSF1 protein at a level that is higher than a baseline level.

A “rejuvenated cell” is an aged cell that has been transiently or stably transfected with a protein or a nucleic acid encoding the protein such that the cell has a transcriptomic profile of a younger cell while still retaining one or more cell identity markers. Thus, a “rejuvenated cell” is an aged cell that has been transiently or stably transfected with an SRSF1 protein or a nucleic acid encoding an SRSF1 protein such that the cell has a transcriptomic profile of a younger cell while still retaining one or more cell identity markers. A “rejuvenated cell” may also be an aged cell that has been transiently or stably transfected with an SLC2A13, RNASEL, WDTC1, and/or NPM1 protein or a nucleic acid encoding an SLC2A13, RNASEL, WDTC1, and/or NPM1 protein such that the cell has a transcriptomic profile of a younger cell while still retaining one or more cell identity markers.

A transcriptomic profile refers to the set of all RNA molecules in one cell or a population of cells. It is sometimes used to refer to all RNAs, or just mRNA, depending on the particular experiment. It differs from the exome in that it includes only those RNA molecules found in a specified cell population, and usually includes the amount or concentration of each RNA molecule in addition to the molecular identities. Methods of obtaining a transcriptomic profile include DNA microarrays and next-generation sequencing technologies such as RNA-Seq. Transcription can also be studied at the level of individual cells by single-cell transcriptomics. There are two general methods of inferring transcriptome sequences. One approach maps sequence reads onto a reference genome, either of the organism itself (whose transcriptome is being studied) or of a closely related species. The other approach, de novo transcriptome assembly, uses software to infer transcripts directly from short sequence reads.

In some embodiments, a transcriptomic profile of a rejuvenated cell becomes more similar to a transcriptomic profile of a young cell. For example, the transcriptomic profile of a rejuvenated cell may comprise an increase or decrease in gene expression (e.g., toward the expected levels in young cells) of one or more genes selected from DVL2, GAS7, PAX6, SLC6A13, VEZT, TMSB10, CYFIP2, KMT2C, EIF4B, CTNNA2, DHX29, LRP6, PFN2, ASIC4, WNT8B, MLLT10, CDH17, GSK3B, EIF2AK1, CETP, FXYD5, SLC9A1, PLEKHG2, EZR, FOXRED2, EIF3D, DAAM1, UGGT2, NUMBL, CYTH2, CBLL1, HSPB1, SFRP4, EIF4H, KDM4C, RAPGEF1, RNF43, ASIC2, EIF4G2, ASIC1, LIN7A, SCNN1A, FAF2, WNT5A, AUP1, KDM3A, EDEM3, WDR77, FBXO2, SSX2IP, ZMIZ2, RASSF8, NRK, RAP2A, VASP, EIF2S2, PRMT1, KDM4B, CGNL1, ASH2L, APOC1, EIF2S3, DIAPH1, KREMEN2, EIF5A, WASF3, SARAF, MEN1, EIF2S1, CTIF, VAV3, WNT2B, LRP4, OS9, WNT10A, KAT7, SCN2A, UGGT1, GYPC, ABI1, FCHSD2, SLC5A6, TET1, SHROOM3, LEF1, WTIP, CARM1, SETDB1, SMYD2, SCN1A, SETD7, ERLIN2, MAL2, PARD3, EIF4EBP2, TCF7L2, SCN3A, APCDD1, DKK2, USP25, TIAM1, FZD1, EIF5B, SLC5A11, LRP5, WNT4, PYGO2, EIF4E3, NFASC, PRDM9, SOX17, DACT1, FRAT1, SCNN1G, CTNNB1, CDH2, RNF139, ATF7IP, JMJD1C, EIF2AK3, JUP, FZD4, SGF29, MYOID, FZD8, EIF3K, KREMEN1, AMER1, UBE2G2, MORF4L1, BCAP31, FNBP1, TET3, FZD9, EVL, AJAP1, PCBP2, SLC9A8, VPS13A, CES1, L1CAM, RNF5, HOTAIR, GET4, EIF4EBP3, and BRK1.

In some embodiments, a rejuvenated cell exhibits increased gene expression of one or more nuclear and/or epigenetic markers compared to a control (e.g., a reference value). For example, the marker may be selected from HP 1 gamma, H3K9me3, lamina support protein LAP2alpha, and SIRT1 protein.

In some embodiments, a rejuvenated cell exhibits increased proteolytic activity compared to a control. For example, the increased proteolytic activity may be measured as increased cell autophagosome formation, increased chymotrypsin-like proteasome activity, or a combination thereof.

In some embodiments, a rejuvenated cell exhibits improved mitochondria health and/or function compared to a control. For example, the improved mitochondria health and function may be measured as increased mitochondria membrane potential, decreased reactive oxygen species (ROS), or a combination thereof.

In some embodiments, a rejuvenated cell exhibits decreased expression of one or more SASP cytokines compared to a control. For example, the SASP cytokines comprise one or more of IL18, IL1A, GROA, IL22, and IL9.

In some embodiments, a rejuvenated cell exhibits reversal of the methylation landscape. Reversal of the methylation landscape may be measured by Horvath clock estimation, for example.

In some embodiments, the control is a young cell or an aged cell or a reference value obtained from a young cell or an aged cell.

In some embodiments, induction of cellular rejuvenation leads to a reduction or inhibition of cellular senescence. For example, senescence-associated β-galactosidase activity of a cell may be decreased. As another example, proteasomal activity of a cell may be decreased.

Herein, an “effective amount” of a SRSF1 protein (or other protein such as SLC2A13, RNASEL, WDTC1, or NPM1) or a nucleic acid encoding the SRSF1 protein (or other protein such as SLC2A13, RNASEL, WDTC1, or NPM1) is an amount sufficient to initiate the reversal of a hallmark of aging, for example, a younger transcriptomic profile (more similar to a younger cell), increased gene expression of one or more nuclear and/or epigenetic markers, increased proteolytic activity, improved mitochondria health and/or function, decreased expression of one or more SASP cytokines, or reversal of the methylation landscape of the cell, compared to a control (e.g., a reference value, for example, obtained from a young cell or an aged cell). An “effective amount” of a SRSF1 protein (or other protein such as SLC2A13, RNASEL, WDTC1, or NPM1) or a nucleic acid encoding the SRSF1 protein (or other protein such as SLC2A13, RNASEL, WDTC1, or NPM1) also include an amount sufficient to decrease SA-β-gal activity and/or proteasomal activity.

Herein, an “effective amount” of an inhibitor of expression of a pro-aging protein (e.g., KAT7, ESR1, MAPK7, KDM6A, and CTNNB1) is an amount sufficient to initiate the reversal of a hallmark of aging, for example, a younger transcriptomic profile (more similar to a younger cell), increased gene expression of one or more nuclear and/or epigenetic markers, increased proteolytic activity, improved mitochondria health and/or function, decreased expression of one or more SASP cytokines, or reversal of the methylation landscape of the cell, compared to a control (e.g., a reference value, for example, obtained from a young cell or an aged cell). An “effective amount” of an inhibitor of expression of a pro-aging protein also includes an amount sufficient to decrease SA-β-gal activity and/or proteasomal activity.

### Cellular Senescence

Cellular senescence is the disruption of cell proliferation and function. During cellular senescence, there is a loss of the ability of the cell to proliferate, although the cell continues to remain viable and metabolically active.

Many cell types undergo cellular senescence following a large number of cycles of cell division. This barrier to further proliferation following many cycles of cell division has been termed replicative senescence. Replicative senescence is thought to be due to shortening of the cell's telomeres with each successive cell division, causing cells to reach a point (their so-called “Hayflick limit”) at which a DNA damage response is triggered, leading ultimately to induction of proliferation arrest and cellular senescence. Cellular senescence can also be induced in the absence of telomere loss or dysfunction. DNA damage may take the form of chromosomal dysfunction such as aneuploidy arising from unequal chromosome segregation during mitosis, DNA strand breaks, or chemical modification of DNA. Cellular senescence may also be induced by a DNA damage response (DDR) which may or may not reflect actual DNA damage.

Cellular senescence, in some embodiments, is characterized by, and may be induced by, changes in chromatin organization that induce changes in gene expression, such as for example, the “senescence-associated secretory phenotype” (“SASP”) in which senescent cells secrete inflammatory cytokines and mitokines that can damage or alter the surrounding tissue. Thus, an SASP is an array of diverse cytokines, chemokines, growth factors, and proteases that are a characteristic feature of senescent cells. Senescent cells are stable, non-dividing cells that are still metabolically active and exhibit the upregulation of a wide range of genes including those that encode secreted proteins, such as inflammatory cytokines, chemokines, extracellular matrix remodeling factors, and growth factors. These secreted proteins function physiologically in the tissue microenvironment, in which they could propagate the stress response and communicate with neighboring cells. This SASP phenotype uncovers the paracrine function of senescent cells, and is an important characteristic that distinguishes senescent cells from non-senescent, cell cycle-arrested cells, such as quiescent cells and terminally differentiated cells. SASP cytokines are cytokines produced specifically by senescent cells to create the senescence-associated secretory phenotype. These cytokines include but are not limited to IL18, IL1A, GROA, IL22, and IL9.

Studies have indicated that cellular senescence is associated with age-related conditions, including thinning of the epidermis, skin wrinkling, hair loss and greying hair, reduction in muscle thickness and muscle strength, increased incidence of inflammation, metabolic disturbances, loss of endurance, and age-associated diseases. In addition, cellular senescence is believed to contribute to difficulties associated with wound healing.

Accordingly, preventing cells from undergoing cellular senescence, or reversing cellular senescence in cells which have undergone cellular senescence, would be advantageous to treat various age-related conditions, would healing, and cosmetic applications.

There are several assays used by researchers for detecting senescence. The colorimetric substrate for β-gal, 5-bromo-4-chloro-3-indolyl-β-D-galactopyranoside, known as x-gal has long been used to detect metabolic activity in cells in vitro. On hydrolysis by β-gal, x-gal is converted into a blue precipitate that can be detected using microscopy. While the x-gal assay is viewed as the “gold standard” method, it is limited in that it is a colorimetric assay. C12FDG is a fluorescent alternative to x-gal. If also functions as a β-gal substrate, but has the drawback of leaking out of the cell within a short period of time. In flow cytometry, a combination of antibody markers such as p16ARF and p21 may be used. An alternative is CellEvent™ Senescence Green Reagent. It offers a sensitive, fluorescent substrate for β-gal that can be used for the detection of senescent cells in flow cytometry assays and imaging applications. It offers the advantage of not only being a fluorescent substrate for β-gal, but does not leak out of cells with time due to its ability to covalently bind to intracellular proteins.

Aspects of the present disclosure are related to contacting a cell with an effective amount of a SRSF1 protein (or other protein such as SLC2A13, RNASEL, WDTC1, or NPM1) or a nucleic acid encoding the SRSF1 protein (or other protein such as SLC2A13, RNASEL, WDTC1, or NPM1), wherein the effective amount is sufficient to decrease cellular senescence of the cell, relative to a control cell. In some embodiments, the effective amount is sufficient to decrease cellular senescence of the cell by at least 20%, at least 30%, at least 40%, or at least 50%. In some embodiments, the effective amount is sufficient to decrease cellular senescence of the cell by at least 50%. In some embodiments, the effective amount is sufficient to decrease cellular senescence of the cell by at least 50%. In some embodiments, the effective amount is sufficient to decrease senescence-associated β-galactosidase activity of the cell, relative to a control. In some embodiments, the effective amount is sufficient to decrease the senescence-associated β-galactosidase activity of the cell by at least 25%, at least 30%, at least 35%, at least 40%, at least 45%, or at least 50%. In some embodiments, the effective amount is sufficient to decrease proteasomal activity of the cell, relative to a control. In some embodiments, the effective amount is sufficient to decrease the proteasomal activity of the cell by at least 25%, at least 30%, at least 40%, at least 45%, or at least 50%.

Aspects of the present disclosure are related to contacting a cell with an effective amount of an inhibitor of expression of one or more pro-aging genes (e.g., KAT7, ESR1, MAPK7, KDM6A, and CTNNB1), wherein the effective amount is sufficient to decrease cellular senescence of the cell, relative to a control cell. In some embodiments, the effective amount is sufficient to decrease cellular senescence of the cell by at least 20%, at least 30%, at least 40%, or at least 50%. In some embodiments, the effective amount is sufficient to decrease cellular senescence of the cell by at least 50%. In some embodiments, the effective amount is sufficient to decrease cellular senescence of the cell by at least 50%. In some embodiments, the effective amount is sufficient to decrease senescence-associated β-galactosidase activity of the cell, relative to a control. In some embodiments, the effective amount is sufficient to decrease the senescence-associated β-galactosidase activity of the cell by at least 25%, at least 30%, at least 35%, at least 40%, at least 45%, or at least 50%. In some embodiments, the effective amount is sufficient to decrease proteasomal activity of the cell, relative to a control. In some embodiments, the effective amount is sufficient to decrease the proteasomal activity of the cell by at least 25%, at least 30%, at least 40%, at least 45%, or at least 50%.

### Senescence-Associated Beta-Galactosidase Activities

Some aspects provide a method for inducing cellular rejuvenation of a cell comprising contacting the cell with an effective amount of a SRSF1 protein (or other protein such as SLC2A13, RNASEL, WDTC1, or NPM1) or a nucleic acid encoding the SRSF1 protein (or other protein such as SLC2A13, RNASEL, WDTC1, NPM1), wherein the effective amount is sufficient to decrease senescence-associated beta-galactosidase (SA-βgal) activity. SA-βgal activity, detectable at pH 6.0, permits the identification of senescent cells in culture and mammalian tissues. SA-βgal activity may be assessed using, for example, a cytochemical protocol suitable for the histochemical detection of individual senescent cells both in culture and tissue biopsies. As another example, a method based on the alkalinization of lysosomes, followed by the use of 5-dodecanoylaminofluorescein di-β-D-galactopyranoside (C12FDG), a fluorogenic substrate for βgal activity, may be used. See, e.g., Debacq-Chainiaux, F et al. Nature Protocols 2009; 4:1798-1806 for exemplary protocols. The cytochemical method is applicable to tissue sections and requires simple reagents and equipment. The fluorescence-based methods have the advantages of being more quantitative and sensitive.

In some embodiments, the effective amount is sufficient to decrease the SA-βgal activity by at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, or at least 100% relative to a control. In some embodiments, the effective amount is sufficient to decrease the SA-βgal activity of the cell by about 50% to about 100%.

Some aspects provide a method for inducing cellular rejuvenation of a cell comprising contacting the cell with an effective amount of an inhibitor of expression of one or more pro-aging proteins (e.g., KAT7, ESR1, MAPK7, KDM6A, and CTNNB1) or expression of one or more nucleic acids encoding the one or more pro-aging proteins, wherein the effective amount is sufficient to decrease senescence-associated beta-galactosidase (SA-βgal) activity. SA-βgal activity, detectable at pH 6.0, permits the identification of senescent cells in culture and mammalian tissues. SA-βgal activity may be assessed using, for example, a cytochemical protocol suitable for the histochemical detection of individual senescent cells both in culture and tissue biopsies. As another example, a method based on the alkalinization of lysosomes, followed by the use of 5-dodecanoylaminofluorescein di-β-D-galactopyranoside (C12FDG), a fluorogenic substrate for βgal activity, may be used. See, e.g., Debacq-Chainiaux, F et al. Nature Protocols 2009; 4:1798-1806 for exemplary protocols. The cytochemical method is applicable to tissue sections and requires simple reagents and equipment. The fluorescence-based methods have the advantages of being more quantitative and sensitive.

In some embodiments, the effective amount is sufficient to decrease the SA-βgal activity by at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, or at least 100% relative to a control. In some embodiments, the effective amount is sufficient to decrease the SA-βgal activity of the cell by about 50% to about 100%.

### Proteasomal Activity

Some aspects provide a method for inducing cellular rejuvenation of a cell comprising contacting the cell with an effective amount of a SRSF1 protein (or other protein such as SLC2A13, RNASEL, WDTC1, or NPM1) or a nucleic acid encoding the SRSF1 protein (or other protein such as SLC2A13, RNASEL, WDTC1, or NPM1), wherein the effective amount is sufficient to decrease the proteasomal activity of the cell. Proteasome activity refers to the degradation of unneeded or damaged proteins by the proteasome, a protein complex, through proteolysis, a chemical reaction that breaks peptide bonds. Chymotrypsin-like proteasome activity is a distinct catalytic activity of the proteasome. In some embodiments, proteasomal activity is measured using fluorescently-labeled peptides. In some embodiments, proteasomal activity is determined by measuring the release of a fluorophore from a peptide substrate. In some embodiments, proteasomal activity is measured by a staining assay. In some embodiments, a staining assay measures cellular features associated with aging. In some embodiments, a staining assay measures the activity of a 20S proteasome. In some embodiments, a staining assay measures that activity of a 26S proteasome.

In some embodiments, the effective amount is sufficient to decrease the proteasomal activity of the cell by at least 25%, at least 30%, at least 35%, at least 40%, at least 45%, or at least 50%, relative to a control. In some embodiments, the effective amount is sufficient to decrease the proteasomal activity of the cells by about 50% to about 100%.

Some aspects provide a method for inducing cellular rejuvenation of a cell comprising contacting the cell with an effective amount of an inhibitor of expression of one or more pro-aging proteins (e.g., KAT7, ESR1, MAPK7, KDM6A, and CTNNB1) or expression of one or more nucleic acids encoding the one or more pro-aging proteins, wherein the effective amount is sufficient to decrease the proteasomal activity of the cell. Proteasome activity refers to the degradation of unneeded or damaged proteins by the proteasome, a protein complex, through proteolysis, a chemical reaction that breaks peptide bonds. Chymotrypsin-like proteasome activity is a distinct catalytic activity of the proteasome. In some embodiments, proteasomal activity is measured using fluorescently-labeled peptides. In some embodiments, proteasomal activity is determined by measuring the release of a fluorophore from a peptide substrate. In some embodiments, proteasomal activity is measured by a staining assay. In some embodiments, a staining assay measures cellular features associated with aging. In some embodiments, a staining assay measures the activity of a 20S proteasome. In some embodiments, a staining assay measures that activity of a 26S proteasome.

In some embodiments, the effective amount is sufficient to decrease the proteasomal activity of the cell by at least 25%, at least 30%, at least 35%, at least 40%, at least 45%, or at least 50%, relative to a control. In some embodiments, the effective amount is sufficient to decrease the proteasomal activity of the cells by about 50% to about 100%.

### Rejuvenated Cells and Methods of Production

Some aspects of the present disclosure are related to methods of inducing rejuvenation of a cell (one or more cells) by overexpression of a SRSF1 protein. Other aspects are related to methods of inducing rejuvenation of a cell (one or more cells) by overexpression of a SLC2A13 protein. Yet other aspects are related to methods of inducing rejuvenation of a cell (one or more cells) by overexpression of a RNASEL protein. Still other aspects are related to methods of inducing rejuvenation of a cell (one or more cells) by overexpression of a WDTC1 protein. Other aspects are related to methods of inducing rejuvenation of a cell (one or more cells) by overexpression of an NPM1 protein. Some aspects are related to methods of inducing rejuvenation of a cell (one or more cells) by contacting the cell with an inhibitor of expression of one or more pro-aging proteins (e.g., KAT7, ESR1, MAPK7, KDM6A, and CTNNB1) or one or more nucleic acids encoding the one or more pro-aging proteins. In some embodiments, functionality is restored in the cell. Functionalities include, for example, mitochondrial function, proteolytic activity, heterochromatin levels, histone methylation, nuclear lamina polypeptides, cytokine secretion, or senescence. In some embodiments, an SRSF1 protein or a nucleic acid (DNA or RNA, e.g., RNA) encoding SRSF1 protein can be used to rejuvenate a variety of cell types, including fibroblasts, hematopoietic stem cells, endothelial cells, chondrocytes, skeletal muscle stem cells, keratinocytes, mesenchymal stem cells and corneal epithelial cells, optionally while retaining cell identity. In some embodiments, an SLC2A13 protein or a nucleic acid (DNA or RNA, e.g., RNA) encoding SLC2A13 protein can be used to rejuvenate a variety of cell types, including fibroblasts, hematopoietic stem cells, endothelial cells, chondrocytes, skeletal muscle stem cells, keratinocytes, mesenchymal stem cells and corneal epithelial cells, optionally while, optionally retaining cell identity. In some embodiments, an RNASEL protein or a nucleic acid (DNA or RNA, e.g., RNA) encoding RNASEL protein can be used to rejuvenate a variety of cell types, including fibroblasts, hematopoietic stem cells, endothelial cells, chondrocytes, skeletal muscle stem cells, keratinocytes, mesenchymal stem cells and corneal epithelial cells while, optionally retaining cell identity. In some embodiments, a WDTC1 protein or a nucleic acid (DNA or RNA, e.g., RNA) encoding WDTC1 protein can be used to rejuvenate a variety of cell types, including fibroblasts, hematopoietic stem cells, endothelial cells, chondrocytes, skeletal muscle stem cells, keratinocytes, mesenchymal stem cells and corneal epithelial cells while, optionally retaining cell identity. In some embodiments, an NPM1 protein or a nucleic acid (DNA or RNA, e.g., RNA) encoding an NPM1 protein can be used to rejuvenate a variety of cell types, including fibroblasts, hematopoietic stem cells, endothelial cells, chondrocytes, skeletal muscle stem cells, keratinocytes, mesenchymal stem cells and corneal epithelial cells while, optionally retaining cell identity.

Some aspects provide methods of inducing rejuvenation of a cell that comprise transfecting a cell with a gene associated with inducing rejuvenation of a cell, thereby inducing rejuvenation of a cell. In some embodiments, a rejuvenated cell is produced.

In some embodiments, a rejuvenated cell had a phenotype or activity profile similar to a young cell. The phenotype or activity profile includes one or more of the transcriptomic profile, gene expression of one or more nuclear and/or epigenetic markers, proteolytic activity, mitochondrial health and function, SASP cytokine expression, and methylation landscape.

In some embodiments, a rejuvenated cell has a transcriptomic profile that is more similar to the transcriptomic profile of young cells. In some embodiments, the transcriptomic profile of a rejuvenated cell includes an increase or decrease in gene expression (e.g., toward the expected levels in young cells) of one or more genes selected from DVL2, GAS7, PAX6, SLC6A13, VEZT, TMSB10, CYFIP2, KMT2C, EIF4B, CTNNA2, DHX29, LRP6, PFN2, ASIC4, WNT8B, MLLT10, CDH17, GSK3B, EIF2AK1, CETP, FXYD5, SLC9A1, PLEKHG2, EZR, FOXRED2, EIF3D, DAAM1, UGGT2, NUMBL, CYTH2, CBLL1, HSPB1, SFRP4, EIF4H, KDM4C, RAPGEF1, RNF43, ASIC2, EIF4G2, ASIC1, LIN7A, SCNN1A, FAF2, WNT5A, AUP1, KDM3A, EDEM3, WDR77, FBXO2, SSX2IP, ZMIZ2, RASSF8, NRK, RAP2A, VASP, EIF2S2, PRMT1, KDM4B, CGNL1, ASH2L, APOC1, EIF2S3, DIAPH1, KREMEN2, EIF5A, WASF3, SARAF, MEN1, EIF2S1, CTIF, VAV3, WNT2B, LRP4, OS9, WNT10A, KAT7, SCN2A, UGGT1, GYPC, ABI1, FCHSD2, SLC5A6, TET1, SHROOM3, LEF1, WTIP, CARM1, SETDB1, SMYD2, SCN1A, SETD7, ERLIN2, MAL2, PARD3, EIF4EBP2, TCF7L2, SCN3A, APCDD1, DKK2, USP25, TIAM1, FZD1, EIF5B, SLC5A11, LRP5, WNT4, PYGO2, EIF4E3, NFASC, PRDM9, SOX17, DACT1, FRAT1, SCNN1G, CTNNB1, CDH2, RNF139, ATF7IP, JMJD1C, EIF2AK3, JUP, FZD4, SGF29, MYO1D, FZD8, EIF3K, KREMEN1, AMER1, UBE2G2, MORF4L1, BCAP31, FNBP1, TET3, FZD9, EVL, AJAP1, PCBP2, SLC9A8, VPS13A, CES1, L1CAM, RNF5, HOTAIR, GET4, EIF4EBP3, and BRK1.

In some embodiments, a rejuvenated cell exhibits increased gene expression of one or more nuclear and/or epigenetic markers compared to a control (e.g., a reference value). In some embodiments, the one or more nuclear and/or epigenetic markers is selected from HP 1 gamma, H3K9me3, lamina support protein LAP2alpha, and SIRT1 protein. In some embodiments, the rejuvenated cell exhibits increased gene expression of HP 1 gamma. In some embodiments, the rejuvenated cell exhibits increased gene expression of H3K9me3. In some embodiments, the rejuvenated cell exhibits increased gene expression of lamina support protein LAP2alpha. In some embodiments, the rejuvenated cell exhibits increased gene expression of SIRT1 protein. In some embodiments, the rejuvenated cell exhibits increased gene expression of HP 1 gamma, H3K9me3, lamina support protein LAP2alpha, and SIRT1 protein.

In some embodiments, a rejuvenated cell has a proteolytic activity that is more similar to the proteolytic activity of young cells. In some embodiments, the proteolytic activity is measured as increased cell autophagosome formation, increased chymotrypsin-like proteasome activity, or a combination thereof. In some embodiments, the proteolytic activity is measured as increased cell autophagosome formation. An autophagosome is a spherical structure with double layer membranes. It is a key structure in macroautophagy, the intracellular degradation system for cytoplasmic contents (e.g., abnormal intracellular proteins, excess or damaged organelles) and also for invading microorganisms. After formation, autophagosomes deliver cytoplasmic components to the lysosomes. The outer membrane of an autophagosome fuses with a lysosome to form an autolysosome. The lysosome's hydrolases degrade the autophagosome-delivered contents and its inner membrane. In some embodiments, the proteolytic activity is measured as increased chymotrypsin-like proteasome activity. In some embodiments, the proteolytic activity is measured as increased cell autophagosome formation and increased chymotrypsin-like proteasome activity.

In some embodiments, the rejuvenated cell exhibits improved mitochondria health and function compared to a control (e.g., a reference value). In some embodiments, improved mitochondria health and function is measured as increased mitochondria membrane potential, decreased reactive oxygen species (ROS), or a combination thereof. In some embodiments, improved mitochondria health and function is measured as increased mitochondria membrane potential. In some embodiments, improved mitochondria health and function is measured as decreased reactive oxygen species (ROS). In some embodiments, improved mitochondria health and function is measured as increased mitochondria membrane potential and decreased reactive oxygen species (ROS).

In some embodiments, a rejuvenated cell exhibits decreased expression of one or more SASP cytokines compared to a control (e.g., a reference value). In some embodiments, the one or more SASP cytokines include IL18, IL1A, GROA, IL22, and IL9. In some embodiments, the rejuvenated cell exhibits decreased expression of IL18. In some embodiments, the rejuvenated cell exhibits decreased expression of IL1A. In some embodiments, the rejuvenated cell exhibits decreased expression of GROA. In some embodiments, the rejuvenated cell exhibits decreased expression of IL22. In some embodiments, the rejuvenated cell exhibits decreased expression of IL9. In some embodiments, the rejuvenated cell exhibits decreased expression of IL18, IL1A, GROA, IL22, and IL9.

In some embodiments, a rejuvenated cell exhibits reversal of the methylation landscape. In some embodiments, the reversal of the methylation landscape is measured by Horvath clock estimation.

In some embodiments, a reference value is obtained from a young cell or an aged cell. It should be understood that any “increase” or “decrease” (e.g., reduce or reduction) of a characteristic and/or function exhibited by a cell is relative to or compared to a control, such as a reference value (e.g., obtained from a young cell or an aged cell).

### Cell Transfection Methods

Aspects of the present disclosure are related to contacting a cell with an effective amount of an SRSF1 protein (or an SLC2A13, RNASEL, WDTC1, or NPM1 protein) or a nucleic acid (e.g., DNA) encoding a SRSF1 protein (or an SLC2A13, RNASEL, WDTC1, or NPM1 protein). In some embodiments, the method comprises delivering to cells the SRSF1 protein. In some embodiments, the method comprises delivering to cells the nucleic acid comprising an open reading frame encoding the SRSF1 protein. In some embodiments, the nucleic acid is delivered on a non-viral vector (e.g., plasmid) or a viral vector (e.g., rAAV vector). In some embodiments, the contacting comprises transfecting the cells (e.g., using electroporation or a chemical transfection agent).

Transfection refers to the uptake of exogenous (e.g., engineered) nucleic acids (e.g., DNA or RNA) by a cell. A cell has been “transfected” when an exogenous nucleic acid has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more engineered nucleic acid into cells. The term refers to both stable and transient uptake of the nucleic acid (e.g., DNA or RNA). For example, transfection can be used for transient uptake of mRNA encoding SRSF1, SLC2A13, RNASEL, WDTC1, and/or NPM1 into cells in need of rejuvenation.

In some embodiments, cells are transfected with an engineered nucleic acid by nucleofection. The term “nucleofection,” as used herein, refers to an electroporation-based transfection method which enables transfer of nucleic acids, such as DNA and RNA, into cells by applying a specific voltage and using specific reagents. See, e.g., Distler et al. (2005) Exp Dermatol, 14 (4): 315-20.

In embodiments, transfecting cells with SRSF1 protein (or an SLC2A13, RNASEL, WDTC1, or NPM1 protein) or a nucleic acid encoding a SRSF1 protein (or an SLC2A13, RNASEL, WDTC1, or NPM1 protein) may be accomplished by a transfection method selected from electroporation, nucleofection, lipofectamine and LT-1 mediated transfection, dextran-mediated transfection, calcium phosphate precipitation, polybrene mediated transfection, encapsulation of the nucleic acid (e.g., mRNA) in liposomes, and direct microinjection. In some embodiments, transfecting cells with protein or nucleic acid is accomplished by lipofectamine and LT-1 mediated transfection. In some embodiments, transfecting cells with protein or nucleic acid is accomplished by dextran-mediated transfection. In some embodiments, transfecting cells with protein or nucleic acid is accomplished by calcium phosphate precipitation. In some embodiments, transfecting cells with protein or nucleic acid is accomplished by polybrene mediated transfection. In some embodiments, transfecting cells with protein or nucleic acid is accomplished by electroporation. In some embodiments, transfecting cells with protein or nucleic acid is accomplished by encapsulation of the mRNAs in liposomes. In some embodiments, transfecting cells with protein or nucleic acid is accomplished by direct microinjection.

In some embodiments, the cell expresses SRSF1 at a level that is higher than a baseline level.

Methods of Inhibiting Protein and/or Nucleic Acid Expression

Aspects of the present disclosure relate, at least in part, to the identification of proteins associated with inhibiting cellular rejuvenation and/or accelerating cellular senescence. In some embodiments, these proteins are termed “pro-aging” proteins. In some embodiments, a pro-aging protein is KAT7. In some embodiments, a pro-aging protein is ESR1. In some embodiments, a pro-aging protein is MAPK7. In some embodiments, a pro-aging protein is KDM6a. In some embodiments, a pro-aging protein is CTNNB1.

Aspects of the present disclosure relate, at least in part, to methods of inhibiting expression of pro-aging protein or expression of nucleic acids encoding pro-aging proteins. In some embodiments, expression of pro-aging proteins or expression of nucleic acids encoding pro-aging proteins is accomplished using a Clustered Regularly Interspaced Short Palindromic Repeats (CRISPR) system. In some embodiments, expression of pro-aging proteins or expression of nucleic acids encoding pro-aging proteins is accomplished using a CRISPR-Cas9 system. In some embodiments, expression of pro-aging proteins or expression of nucleic acids encoding pro-aging proteins is accomplished using a CRISPR-interference (CRISPRi) system. In some embodiments, inhibition of expression of pro-aging proteins or expression of nucleic acids encoding pro-aging proteins is accomplished using a CRISPRoff system. In some embodiments, inhibition of expression of pro-aging proteins or expression of nucleic acids encoding pro-aging proteins is accomplished using an shRNA. In some embodiments, inhibition of expression of pro-aging proteins or expression of nucleic acids encoding pro-aging proteins is accomplished using an siRNA. In some embodiments, inhibition of expression of pro-aging proteins or expression of nucleic acids encoding pro-aging proteins is accomplished using an miRNA. In some embodiments, inhibition of expression of pro-aging proteins or expression of nucleic acids encoding pro-aging proteins is accomplished using a small molecule inhibitor. In some embodiments, inhibition of expression of pro-aging proteins or expression of nucleic acids encoding pro-aging proteins is accomplished using an antibody.

In some embodiments, the CRISPR system is a genome editing system. In some embodiments, a CRISPR guide RNA (gRNA) is coupled to a CRISPR endonuclease. In some embodiments, the gRNA guides the endonuclease to a site in the genome. In some embodiments, the endonuclease cleaves the genomic DNA. In some embodiments, cleaving genomic DNA inhibits expression of the gene. In some embodiments, inhibiting expression of the gene inhibits expression of the protein encoded by the gene. In some embodiments, the endonuclease is a Cas9 endonuclease. In some embodiments, the endonuclease is a Cas12a endonuclease. In some embodiments, the endonuclease is a Cas13 endonuclease. In some embodiments, the CRISPR system comprises a gRNA and an endonuclease. In some embodiments, the CRISPR-Cas9 system comprises a gRNA and a Cas9 endonuclease. See, e.g., Doudna J. A., Charpentier E. Science 346, 6213 (2014), the entire contents of which are hereby incorporated by reference.

In some embodiments, the CRISPR system is a CRISPRi system. In some embodiments, a CRISPRi system comprises a gRNA and a nuclease-dead endonuclease. In some embodiments, the nuclease-dead endonuclease is a nuclease-dead Cas9 (dCas9). In some embodiments, a nuclease-dead endonuclease cannot cleave DNA. In some embodiments, the nuclease-dead endonuclease blocks transcription of a target gene. In some embodiments, the target gene is a gene determined by the gRNA. In some embodiments, the nuclease-dead endonuclease blocks transcription by steric hindrance. In some embodiments, CRISPRi reduces expression of the target gene and the protein encoded by the target gene, compared to a control. See, e.g., Larson H. M., et al. Nature Protocols 8, 2180-2196 (2013), the entire contents of which are hereby incorporated by reference.

In some embodiments, the CRISPR system is a CRISPRoff system. In some embodiments, a CRISPRoff system comprises a gRNA and a nuclease-dead endonuclease. In some embodiments, the nuclease-dead endonuclease is a nuclease-dead Cas9 (dCas9). In some embodiments, a nuclease-dead endonuclease cannot cleave DNA. In some embodiments, the CRISPRoff system initiates methylation at a target site in the genome. In some embodiments, methylation blocks transcription of a target gene. In some embodiments, CRISPRoff reduces expression of the target gene and the protein encoded by the target gene, compared to a control. See, e.g., Nunez J. K., et al. Cell 184, 2503-2519 (2021), the entire contents of which are hereby incorporated by reference.

In some embodiments, inhibition of expression of pro-aging proteins or expression of nucleic acids encoding pro-aging proteins is accomplished using a small hairpin RNA (shRNA). In some embodiments, shRNA is inserted into a cell and converted to a hairpin RNA structure. In some embodiments, the shRNA binds to mRNA and blocks mRNA translation. In some embodiments, the mRNA is degraded after binding to the shRNA. In some embodiments, inhibition of expression of pro-aging proteins or expression of nucleic acids encoding pro-aging proteins is accomplished using an siRNA. In some embodiments, siRNA is a RNA duplex that is designed to target a specific mRNA sequence. In some embodiments, siRNA binds to mRNA and facilitates degradation of the mRNA. In some embodiments, inhibition of expression of pro-aging proteins or expression of nucleic acids encoding pro-aging proteins is accomplished using a small molecule inhibitor. In some embodiments, a small molecule inhibitor targets signaling molecules. In some embodiments, a small molecule inhibitor blocks transcription. In some embodiments, a small molecule inhibitor blocks translation. In some embodiments, a small molecule inhibitor targets a DNA-binding domain.

As will be understood by a person having ordinary skill in the art, any of the inhibition methods described herein can be delivered to a cell using any one of the delivery systems described herein, including, but not limited to, a viral or non-viral vector.

### Cell Types

The methods, in some aspects, are used to induce cellular rejuvenation in cells. In some embodiments, the methods provided herein may be applied to any type of cell in need of rejuvenation. Cells may be intact live cells, naturally occurring or modified. A cell may be isolated from other cells, mixed with other cells in a culture, or within a tissue (partial or intact) or an organism. The methods described herein can be performed, for example, on a sample comprising a single cell, a population of cells, or a tissue or organ comprising cells. The methods can also be used to deliver nucleic acids or proteins to cells in vivo. The cells chosen for rejuvenation, in some embodiments, depends on the desired therapeutic effect for treating an age-related disease or condition.

In some embodiments, a cell is a mammalian cell (e.g., cell derived from a mammalian subject suitable for transplantation into the same or a different subject). In some embodiments, a cell is a human cell. In some embodiments, a cell is from an elderly subject.

A cell may be xenogeneic, autologous, or allogeneic. A cell can be a primary cell obtained directly from a mammalian subject. The cell may also be a cell derived from the culture and expansion of a cell obtained from a subject. In some embodiments, the cell has been genetically engineered to express SRSF1 protein and/or a nucleic acid encoding SRSF1 protein. In some embodiments, the cell has been genetically engineered to express SLC2A13 protein and/or a nucleic acid encoding SLC2A13 protein. In some embodiments, the cell has been genetically engineered to express RNASEL protein and/or a nucleic acid encoding RNASEL protein. In some embodiments, the cell has been genetically engineered to express WDTC1 protein and/or a nucleic acid encoding WDTC1 protein. In some embodiments, the cell has been genetically engineered to express an NPM1 protein and/or a nucleic acid encoding an NPM1 protein.

The methods, in some aspects, comprising contacting (e.g., transfecting) a cell with a therapeutically effective amount of a SRSF1 protein or a nucleic acid encoding the SRSF1 protein. The methods, in other aspects, comprising contacting (e.g., transfecting) a cell with a therapeutically effective amount of a SLC2A13 protein or a nucleic acid encoding the SLC2A13 protein. The methods, in yet other aspects, comprising contacting (e.g., transfecting) a cell with a therapeutically effective amount of a RNASEL protein or a nucleic acid encoding the RNASEL protein. The methods, in still other aspects, comprising contacting (e.g., transfecting) a cell with a therapeutically effective amount of a WDTC1 protein or a nucleic acid encoding the WDTC1 protein. The methods, in still other aspects, comprise contacting (e.g., transfecting) a cell with a therapeutically effective amount of an NPM1 protein or a nucleic acid encoding the protein. In some embodiments, a cell is selected from fibroblasts, hematopoietic stem cells, endothelial cells, chondrocytes, skeletal muscle stem cells, keratinocytes, mesenchymal stem cells and corneal epithelial cells. In some embodiments, a cell is a fibroblast. In some embodiments, a cell is a hematopoietic stem cell. In some embodiments, a cell is an endothelial cell. In some embodiments, a cell is a chondrocyte. In some embodiments, a cell is a skeletal muscle stem cell. In some embodiments, a cell is a keratinocyte. In some embodiments, a cell is a mesenchymal stem cell. In some embodiments, a cell is a corneal epithelial cell. In some embodiments, a cell is a cardiomyocyte.

A fibroblast is a type of cell that contributes to the formation of connective tissue, a fibrous cellular material that supports and connects other tissues or organs in the body. Fibroblasts secrete collagen proteins that help maintain the structural framework of tissues. Dermal fibroblasts are the main cell type present in skin connective tissue (dermis). Fibroblasts interact with epidermal cells during hair development and in interfollicular skin. Moreover, they play an essential role during cutaneous wound healing and in bioengineering of skin. Detailed procedures for establishing and maintaining primary cultures of adult human dermal fibroblasts are known (see, e.g., Kisiel et al. Methods Mol Biol. 2019; 1993:71-78).

In some embodiments, a rejuvenated fibroblast exhibits a transcriptomic profile similar to a transcriptomic profile of young fibroblasts. In some embodiments, a rejuvenated fibroblast exhibits an increased gene expression of one or more nuclear and/or epigenetic markers compared to a control (e.g., a reference value) as described above. In some embodiments, the rejuvenated fibroblasts have a proteolytic activity that is more similar to the proteolytic activity of young cells as described above. In some embodiments, a rejuvenated fibroblast exhibits improved mitochondria health and function compared to a control (e.g., a reference value) as described above. In some embodiments, a rejuvenated fibroblast exhibits a reversal of the methylation landscape.

In some embodiments, a rejuvenated endothelial cell exhibits a transcriptomic profile similar to a transcriptomic profile of young endothelial cells. In some embodiments, a rejuvenated endothelial cell exhibits increased gene expression of one or more nuclear and/or epigenetic markers compared to a control (e.g., a reference value) as described above. In some embodiments, rejuvenated endothelial cells have a proteolytic activity that is more similar to the proteolytic activity of young cells as described above. In some embodiments, a rejuvenated endothelial cell exhibits improved mitochondria health and function compared to a control (e.g., a reference value) as described above. In some embodiments, a rejuvenated endothelial cell exhibits a reversal of the methylation landscape.

In some embodiments, a rejuvenated chondrocyte exhibits reduced expression of inflammatory factors and/or and increased ATP and collagen metabolism. In some embodiments, the inflammatory factors include RANKL, iNOS2, IL6, IFNa, MCP3 and MIP1A. In some embodiments, a rejuvenated chondrocyte exhibits reduced expression of RANKL. In some embodiments, a rejuvenated chondrocyte exhibits reduced expression of iNOS2. In some embodiments, a rejuvenated chondrocyte exhibits reduced expression of IL6. In some embodiments, a rejuvenated chondrocyte exhibits reduced expression of IFNa. In some embodiments, a rejuvenated chondrocyte exhibits reduced expression of MCP3. In some embodiments, a rejuvenated chondrocyte exhibits reduced expression of MIP1A. In some embodiments, a rejuvenated chondrocyte exhibits reduced expression of RANKL, iNOS2, IL6, IFNa, MCP3 and MIP1A. In some embodiments, a rejuvenated chondrocyte exhibits increased ATP and collagen metabolism. In some embodiments, ATP and collagen metabolism is measured by one or more of increased ATP levels, decreased ROS and increased SOD2 expression, increased COL2A1 expression and overall proliferation by the chondrocyte. In some embodiments, ATP and collagen metabolism is measured by increased ATP levels. In some embodiments, ATP and collagen metabolism is measured by decreased ROS and increased SOD2 expression. In some embodiments, ATP and collagen metabolism is measured by increased COL2A1 expression and overall proliferation by the chondrocyte.

In some embodiments, a rejuvenated skeletal muscle stem cell exhibits higher proliferative capacity, enhanced ability to differentiate into myoblasts and muscle fibers, restored lower kinetics of activation from quiescence, ability to rejuvenate the muscular microniche, restore youthful force in the muscle, or a combination thereof.

In some embodiments, a rejuvenated keratinocytes exhibit higher proliferative capacity, reduced inflammatory phenotype, lower RNAKL and INOS2 expression, reduced expression of cytokines MIP1 A, IL6, IFNa, MCP3, increased ATP, increased levels of SOD2 and COL2A1 expression.

In some embodiments, a rejuvenated mesenchymal stem cell exhibits reduction in senescence parameters, increased cell proliferation, and/or a decrease in ROS levels. In some embodiments, a rejuvenated mesenchymal stem cell exhibits reduction in senescence parameters. In some embodiments, the senescence parameters include pl6 expression, p21 expression and positive SA Gal staining. In some embodiments, a rejuvenated mesenchymal stem cell exhibits increased cell proliferation. In some embodiments, a rejuvenated mesenchymal stem cell exhibits a decrease in ROS levels. In some embodiments, a rejuvenated mesenchymal stem cell exhibits reduction in senescence parameters, increased cell proliferation, and a decrease in ROS levels.

In some embodiments, a rejuvenated corneal epithelial cell exhibits a reduction in senescence parameters. In some embodiments, the senescence parameters include one or more of expression of p21, expression of p16, mitochondria biogenesis PGC1a, and expression of inflammatory factor IL8. In some embodiments, the senescence parameters include p21. In some embodiments, the senescence parameters include expression of p16. In some embodiments, the senescence parameters include mitochondria biogenesis PGC1a. In some embodiments, the senescence parameters include expression of inflammatory factor IL8. In some embodiments, the senescence parameters include one expression of p21, expression of p16, mitochondria biogenesis PGC1a, and expression of inflammatory factor IL8.

In some embodiments, a rejuvenated cardiomyocyte exhibits a reduction in senescence parameters. In some embodiments, the senescence parameters include expression of p16INK4a, or cyclin-dependent kinase inhibitors (CDKIs), such as p21Cip1 and p27Kip1, and the activation of the DNA damage response pathway. In some embodiments, the senescence parameters include expression of p16INK4a. In some embodiments, the senescence parameters include expression of CDKIs. In some embodiments, the senescence parameters include activation of the DNA damage response pathway.

In some embodiments, the cell is a stem cell. In some embodiments, the cell is a hematopoietic stem cell. A stem cell is a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells (iPSCs) are a type of pluripotent stem cell derived from adult cells that have been reprogrammed into an embryonic-like pluripotent state. Induced pluripotent stem cells can be derived, for example, from adult somatic cells such as skin or blood cells.

### Therapies

Aspects of the present disclosure relate to methods and compositions for delivering or administering any one of the proteins or nucleic acids described herein to a subject in need thereof.

### Subjects

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

A subject, in some embodiments, is a human subject. In some embodiments, a subject is a young adult. A young adult subject is between the ages of 18 and 44 years old (including 18 and 44 years old).

In some embodiments, a subject is a middle-aged subject. A middle-aged subject may be between the ages of 45 and 65 years old (including 45 and 65 years old). In some embodiments, a middle-aged subject is between the ages of 50 and 65 years old or between the ages of 55 and 65 years old.

In some embodiments, a subject is an elderly subject. An elderly subject may be older than 65 years old. In some embodiments, an elderly subject is between the ages of 70 and 85 years old or between the ages of 75 and 85 years old.

In some embodiments, a subject is at least 50 years old. In some embodiments, a subject is at least 55 years old. In some embodiments, a subject is at least 60 years old. In some embodiments, a subject is at least 65 years old. In some embodiments, a subject is at least 70 years old. In some embodiments, a subject is at least 75 years old.

### Formulations

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit. Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

The proteins and nucleic acids described herein may be formulated for a particular route of administration, which may depend, for example, on an intended therapy. In some embodiments, an SRSF1 protein (or SLC2A13, RNASEL, WDTC1, or NPM1 protein) or a nucleic acid encoding the protein may be formulated for topical delivery. In some embodiments, an SRSF1 protein (or SLC2A13, RNASEL, WDTC1, or NPM1 protein) or a nucleic acid encoding the protein is formulated for subcutaneous delivery. In some embodiments, an SRSF1 protein (or SLC2A13, RNASEL, WDTC1, or NPM1 protein) or a nucleic acid encoding the protein is formulated for intravenous delivery. In some embodiments, an SRSF1 protein (or SLC2A13, RNASEL, WDTC1, or NPM1 protein) or a nucleic acid encoding the protein is formulated for intramuscular delivery. The formulation, in some embodiments, includes an mRNA encoding an SRSF1 protein (or SLC2A13, RNASEL, WDTC1, or NPM1 protein) and a lipid nanoparticle (LNP) or other lipid-based delivery system. An SRSF1 protein (or SLC2A13, RNASEL, WDTC1, or NPM1 protein) or a nucleic acid encoding the protein, in other embodiments, is formulated for delivery via electroporation.

A protein, nucleic acid, or inhibitor of a protein and/or nucleic acid may be formulated, in some embodiments, with a pharmaceutically acceptable excipient, which an excipient that causes no significant adverse toxicological effects to a subject, such as a human subject.

### Routes of Administration

The route of administration of the proteins, nucleic acids, and inhibitors or protein and/or nucleic acid expression described herein may vary depending on how they are formulated. Non-limiting examples of routes of administration include, topical, oral, nasal, intravenous, intramuscular, subcutaneous, and intraperitoneal.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The term “administer,” “administering,” or “administration” refers to implanting, absorbing, ingesting, injecting, inhaling, or otherwise introducing a compound described herein, or a composition thereof, in or on a subject.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

### Additional Embodiments Relating to SLC2A13

1. A method of inducing cellular rejuvenation of a cell comprising contacting the cell with an effective amount of an SLC2A13 protein or a nucleic acid encoding the SLC2A13 protein.
2. A method of inducing cellular rejuvenation of a cell in a subject comprising administering to the subject an effective amount of an SLC2A13 protein or a nucleic acid encoding the SLC2A13 protein.
3. The method of embodiment 1 or 2, wherein the effective amount is sufficient to decrease cellular senescence of the cell, relative to a control.
4. The method of embodiment 3, wherein the effective amount is sufficient to decrease cellular senescence of the cell by at least 20%, at least 30%, at least 40%, or at least 50%.
5. The method of embodiment 4, wherein the effective amount is sufficient to decrease cellular senescence of the cell by at least 50%.
6. The method of any one of embodiments 3-5, wherein the effective amount is sufficient to decrease senescence-associated β-galactosidase activity of the cell, relative to a control.
7. The method of embodiment 6, wherein the effective amount is sufficient to decrease the senescence-associated β-galactosidase activity of the cell by at least 25%, at least 30%, t least 35%, at least 40%, at least 45%, or at least 50%.
8. The method of any one of embodiments 3-7, wherein the effective amount is sufficient to decrease proteasomal activity of the cell, relative to a control.
9. The method of embodiment 8, wherein the effective amount is sufficient to decrease the proteasomal activity of the cell by at least 25%, at least 30%, at least 40%, at least 45%, or at least 50%.
10. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
11. The method of embodiment 10, wherein the effective amount is sufficient to induce an average cellular rejuvenation of at least 25 years.
12. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
13. The method of embodiment 12, wherein the cells are fibroblasts.
14. The method of embodiment 13, wherein the fibroblasts are human dermal fibroblasts.
15. The method of any one of the preceding embodiments, wherein the nucleic acid comprises a heterologous promoter operably linked to the open reading frame.
16. The method of embodiment 15, wherein the heterologous promoter is an inducible promoter.
17. The method of any one of the preceding embodiments, comprising delivering to cells the SLC2A13 protein.
18. The method of any one of the preceding embodiments, comprising delivering to cells the nucleic acid comprising an open reading frame encoding the SLC2A13 protein.
19. The method of any one of the preceding embodiments, wherein the nucleic acid is delivered on a non-viral vector or a viral vector.
20. The method of any one of the preceding embodiments, wherein the contacting comprises transfecting the cells.
21. A method of inducing cellular rejuvenation of a cell comprising overexpressing in the cell an effective amount of an SLC2A13 protein.
22. The method of embodiment 21, wherein the method comprises activating expression or activity of endogenous SLC2A13 protein at a level that is higher than a baseline level.

### Additional Embodiments Relating to RNASEL

1. A method of inducing cellular rejuvenation of a cell comprising contacting the cell with an effective amount of an RNASEL protein or a nucleic acid encoding the RNASEL protein.
2. A method of inducing cellular rejuvenation of a cell in a subject comprising administering to the subject an effective amount of an RNASEL protein or a nucleic acid encoding the RNASEL protein.
3. The method of embodiment 1 or 2, wherein the effective amount is sufficient to decrease cellular senescence of the cell, relative to a control.
4. The method of embodiment 3, wherein the effective amount is sufficient to decrease cellular senescence of the cell by at least 20%, at least 30%, at least 40%, or at least 50%.
5. The method of embodiment 4, wherein the effective amount is sufficient to decrease cellular senescence of the cell by at least 50%.
6. The method of any one of embodiments 3-5, wherein the effective amount is sufficient to decrease senescence-associated β-galactosidase activity of the cell, relative to a control.
7. The method of embodiment 6, wherein the effective amount is sufficient to decrease the senescence-associated β-galactosidase activity of the cell by at least 25%, at least 30%, t least 35%, at least 40%, at least 45%, or at least 50%.
8. The method of any one of embodiments 3-7, wherein the effective amount is sufficient to decrease proteasomal activity of the cell, relative to a control.
9. The method of embodiment 8, wherein the effective amount is sufficient to decrease the proteasomal activity of the cell by at least 25%, at least 30%, at least 40%, at least 45%, or at least 50%.
10. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
11. The method of embodiment 10, wherein the effective amount is sufficient to induce an average cellular rejuvenation of at least 25 years.
12. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
13. The method of embodiment 12, wherein the cells are fibroblasts.
14. The method of embodiment 13, wherein the fibroblasts are human dermal fibroblasts.
15. The method of any one of the preceding embodiments, wherein the nucleic acid comprises a heterologous promoter operably linked to the open reading frame.
16. The method of embodiment 15, wherein the heterologous promoter is an inducible promoter.
17. The method of any one of the preceding embodiments, comprising delivering to cells the RNASEL protein.
18. The method of any one of the preceding embodiments, comprising delivering to cells the nucleic acid comprising an open reading frame encoding the RNASEL protein.
19. The method of any one of the preceding embodiments, wherein the nucleic acid is delivered on a non-viral vector or a viral vector.
20. The method of any one of the preceding embodiments, wherein the contacting comprises transfecting the cells.
21. A method of inducing cellular rejuvenation of a cell comprising overexpressing in the cell an effective amount of an RNASEL protein.
22. The method of embodiment 21, wherein the method comprises activating expression or activity of endogenous RNASEL protein at a level that is higher than a baseline level.

### Additional Embodiments Relating to WDTC1

1. A method of inducing cellular rejuvenation of a cell comprising contacting the cell with an effective amount of an WDTC1 protein or a nucleic acid encoding the WDTC1 protein.
2. A method of inducing cellular rejuvenation of a cell in a subject comprising administering to the subject an effective amount of an WDTC1 protein or a nucleic acid encoding the WDTC1 protein.
3. The method of embodiment 1 or 2, wherein the effective amount is sufficient to decrease cellular senescence of the cell, relative to a control.
4. The method of embodiment 3, wherein the effective amount is sufficient to decrease cellular senescence of the cell by at least 20%, at least 30%, at least 40%, or at least 50%.
5. The method of embodiment 4, wherein the effective amount is sufficient to decrease cellular senescence of the cell by at least 50%.
6. The method of any one of embodiments 3-5, wherein the effective amount is sufficient to decrease senescence-associated β-galactosidase activity of the cell, relative to a control.
7. The method of embodiment 6, wherein the effective amount is sufficient to decrease the senescence-associated β-galactosidase activity of the cell by at least 25%, at least 30%, t least 35%, at least 40%, at least 45%, or at least 50%.
8. The method of any one of embodiments 3-7, wherein the effective amount is sufficient to decrease proteasomal activity of the cell, relative to a control.
9. The method of embodiment 8, wherein the effective amount is sufficient to decrease the proteasomal activity of the cell by at least 25%, at least 30%, at least 40%, at least 45%, or at least 50%.
10. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
11. The method of embodiment 10, wherein the effective amount is sufficient to induce an average cellular rejuvenation of at least 25 years.
12. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
13. The method of embodiment 12, wherein the cells are fibroblasts.
14. The method of embodiment 13, wherein the fibroblasts are human dermal fibroblasts.
15. The method of any one of the preceding embodiments, wherein the nucleic acid comprises a heterologous promoter operably linked to the open reading frame.
16. The method of embodiment 15, wherein the heterologous promoter is an inducible promoter.
17. The method of any one of the preceding embodiments, comprising delivering to cells the WDTC1 protein.
18. The method of any one of the preceding embodiments, comprising delivering to cells the nucleic acid comprising an open reading frame encoding the WDTC1 protein.
19. The method of any one of the preceding embodiments, wherein the nucleic acid is delivered on a non-viral vector or a viral vector.
20. The method of any one of the preceding embodiments, wherein the contacting comprises transfecting the cells.
21. A method of inducing cellular rejuvenation of a cell comprising overexpressing in the cell an effective amount of an WDTC1 protein.
22. The method of embodiment 21, wherein the method comprises activating expression or activity of endogenous WDTC1 protein at a level that is higher than a baseline level.

### Additional Embodiments Relating to NPM1

1. A method of inducing cellular rejuvenation of a cell comprising contacting the cell with an effective amount of an NPM1 protein or a nucleic acid encoding the NPM1 protein.
2. A method of inducing cellular rejuvenation of a cell in a subject comprising administering to the subject an effective amount of an NPM1 protein or a nucleic acid encoding the NPM1 protein.
3. The method of embodiment 1 or 2, wherein the effective amount is sufficient to decrease cellular senescence of the cell, relative to a control.
4. The method of embodiment 3, wherein the effective amount is sufficient to decrease cellular senescence of the cell by at least 20%, at least 30%, at least 40%, or at least 50%.
5. The method of embodiment 4, wherein the effective amount is sufficient to decrease cellular senescence of the cell by at least 50%.
6. The method of any one of embodiments 3-5, wherein the effective amount is sufficient to decrease senescence-associated β-galactosidase activity of the cell, relative to a control.
7. The method of embodiment 6, wherein the effective amount is sufficient to decrease the senescence-associated β-galactosidase activity of the cell by at least 25%, at least 30%, t least 35%, at least 40%, at least 45%, or at least 50%.
8. The method of any one of embodiments 3-7, wherein the effective amount is sufficient to decrease proteasomal activity of the cell, relative to a control.
9. The method of embodiment 8, wherein the effective amount is sufficient to decrease the proteasomal activity of the cell by at least 25%, at least 30%, at least 40%, at least 45%, or at least 50%.
10. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
11. The method of embodiment 10, wherein the effective amount is sufficient to induce an average cellular rejuvenation of at least 25 years.
12. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
13. The method of embodiment 12, wherein the cells are fibroblasts.
14. The method of embodiment 13, wherein the fibroblasts are human dermal fibroblasts.
15. The method of any one of the preceding embodiments, wherein the nucleic acid comprises a heterologous promoter operably linked to the open reading frame.
16. The method of embodiment 15, wherein the heterologous promoter is an inducible promoter.
17. The method of any one of the preceding embodiments, comprising delivering to cells the NPM1 protein.
18. The method of any one of the preceding embodiments, comprising delivering to cells the nucleic acid comprising an open reading frame encoding the NPM1 protein.
19. The method of any one of the preceding embodiments, wherein the nucleic acid is delivered on a non-viral vector or a viral vector.
20. The method of any one of the preceding embodiments, wherein the contacting comprises transfecting the cells.
21. A method of inducing cellular rejuvenation of a cell comprising overexpressing in the cell an effective amount of an NPM1 protein.
22. The method of embodiment 21, wherein the method comprises activating expression or activity of endogenous NPM1 protein at a level that is higher than a baseline level.

### Additional Embodiments Relating to Anti-Aging Proteins

1. A method of inducing cellular rejuvenation of a cell comprising contacting the cell with an effective amount of one or more anti-aging proteins or one or more nucleic acids encoding the one or more anti-aging proteins.
2. A method of inducing cellular rejuvenation of a cell in a subject comprising administering to the subject an effective amount of one or more anti-aging proteins or one or more nucleic acids encoding the one or more anti-aging proteins.
3. The method of embodiment 1 or 2, wherein the one or more anti-aging proteins is selected from the group consisting of: SRSF1, SLC2A13, RNASEL, WDTC1, and NPM1.
4. The method of any one of the preceding embodiments, wherein the effective amount is sufficient to decrease cellular senescence of the cell, relative to a control.
5. The method of embodiment 4, wherein the effective amount is sufficient to decrease cellular senescence of the cell by at least 20%, at least 30%, at least 40%, or at least 50%.
6. The method of embodiment 5, wherein the effective amount is sufficient to decrease cellular senescence of the cell by at least 50%.
7. The method of any one of embodiments 4-6, wherein the effective amount is sufficient to decrease senescence-associated β-galactosidase activity of the cell, relative to a control.
8. The method of embodiment 7, wherein the effective amount is sufficient to decrease the senescence-associated β-galactosidase activity of the cell by at least 25%, at least 30%, t least 35%, at least 40%, at least 45%, or at least 50%.
9. The method of any one of embodiments 4-8, wherein the effective amount is sufficient to decrease proteasomal activity of the cell, relative to a control.
10. The method of embodiment 9, wherein the effective amount is sufficient to decrease the proteasomal activity of the cell by at least 25%, at least 30%, at least 40%, at least 45%, or at least 50%.
11. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
12. The method of embodiment 11, wherein the effective amount is sufficient to induce an average cellular rejuvenation of at least 25 years.
13. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
14. The method of embodiment 13, wherein the cells are fibroblasts.
15. The method of embodiment 14, wherein the fibroblasts are human dermal fibroblasts.
16. The method of any one of the preceding embodiments, wherein the one or more nucleic acids comprise one or more heterologous promoters operably linked to one or more open reading frames.
17. The method of embodiment 16, wherein the one or more heterologous promoters are inducible promoters.
18. The method of any one of the preceding embodiments, comprising delivering to cells the one or more anti-aging proteins.
19. The method of any one of the preceding embodiments, comprising delivering to cells the one or more nucleic acids comprising one or more open reading frames encoding the one or more anti-aging proteins.
20. The method of any one of the preceding embodiments, wherein the one or more nucleic acid is delivered on a non-viral vector or a viral vector.
21. The method of any one of the preceding embodiments, wherein the contacting comprises transfecting the cells.
22. A method of inducing cellular rejuvenation of a cell comprising overexpressing in the cell an effective amount of one or more anti-aging proteins.
23. The method of embodiment 22, wherein the method comprises activating expression or activity of the endogenous one or more anti-aging proteins at a level that is higher than a baseline level.

### Additional Embodiments Relating to KAT7

1. A method of inducing cellular rejuvenation of a cell comprising contacting the cell with an effective amount of an inhibitor of KAT7 protein expression or expression of a nucleic acid encoding the KAT7 protein.
2. A method of inducing cellular rejuvenation of a cell in a subject comprising administering to the subject an effective amount of an inhibitor of KAT7 protein expression or expression of a nucleic acid encoding the KAT7 protein.
3. The method of embodiment 1 or 2, wherein the inhibitor is a CRSPR-Cas9 system, a CRISPRi system, a CRISPRoff system, an shRNA, an siRNA, or a small molecule inhibitor.
4. The method of any one of the preceding embodiments, wherein the effective amount is sufficient to decrease cellular senescence of the cell, relative to a control.
5. The method of embodiment 4, wherein the effective amount is sufficient to decrease cellular senescence of the cell by at least 20%, at least 30%, at least 40%, or at least 50%.
6. The method of embodiment 5, wherein the effective amount is sufficient to decrease cellular senescence of the cell by at least 50%.
7. The method of any one of embodiments 4-6, wherein the effective amount is sufficient to decrease senescence-associated β-galactosidase activity of the cell, relative to a control.
8. The method of embodiment 7, wherein the effective amount is sufficient to decrease the senescence-associated β-galactosidase activity of the cell by at least 25%, at least 30%, t least 35%, at least 40%, at least 45%, or at least 50%.
9. The method of any one of embodiments 4-8, wherein the effective amount is sufficient to decrease proteasomal activity of the cell, relative to a control.
10. The method of embodiment 9, wherein the effective amount is sufficient to decrease the proteasomal activity of the cell by at least 25%, at least 30%, at least 40%, at least 45%, or at least 50%.
11. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
12. The method of embodiment 11, wherein the effective amount is sufficient to induce an average cellular rejuvenation of at least 25 years.
13. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
14. The method of embodiment 13, wherein the cells are fibroblasts.
15. The method of embodiment 14, wherein the fibroblasts are human dermal fibroblasts.
16. The method of any one of the preceding embodiments, wherein the nucleic acid comprises a heterologous promoter operably linked to the open reading frame.
17. The method of embodiment 16, wherein the heterologous promoter is an inducible promoter.
18. The method of any one of the preceding embodiments, comprising delivering to cells the inhibitor of KAT7 protein expression.
19. The method of any one of the preceding embodiments, comprising delivering to cells the inhibitor of expression of the nucleic acid that encodes the KAT7 protein.
20. The method of any one of the preceding embodiments, wherein the inhibitor is delivered on a non-viral vector or a viral vector.
21. The method of any one of the preceding embodiments, wherein the contacting comprises transfecting the cells.
22. The method of any one of the preceding embodiments, wherein the method comprises reducing expression or activity of endogenous KAT7 protein at a level that is lower than a baseline level.

### Additional Embodiments Relating to ESR1

1. A method of inducing cellular rejuvenation of a cell comprising contacting the cell with an effective amount of an inhibitor of ESR1 protein expression or expression of a nucleic acid encoding the ESR1 protein.
2. A method of inducing cellular rejuvenation of a cell in a subject comprising administering to the subject an effective amount of an inhibitor of ESR1 protein expression or expression of a nucleic acid encoding the ESR1 protein.
3. The method of embodiment 1 or 2, wherein the inhibitor is a CRSPR-Cas9 system, a CRISPRi system, a CRISPRoff system, an shRNA, an siRNA, or a small molecule inhibitor.
4. The method of any one of the preceding embodiments, wherein the effective amount is sufficient to decrease cellular senescence of the cell, relative to a control.
5. The method of embodiment 4, wherein the effective amount is sufficient to decrease cellular senescence of the cell by at least 20%, at least 30%, at least 40%, or at least 50%.
6. The method of embodiment 5, wherein the effective amount is sufficient to decrease cellular senescence of the cell by at least 50%.
7. The method of any one of embodiments 4-6, wherein the effective amount is sufficient to decrease senescence-associated β-galactosidase activity of the cell, relative to a control.
8. The method of embodiment 7, wherein the effective amount is sufficient to decrease the senescence-associated β-galactosidase activity of the cell by at least 25%, at least 30%, t least 35%, at least 40%, at least 45%, or at least 50%.
9. The method of any one of embodiments 4-8, wherein the effective amount is sufficient to decrease proteasomal activity of the cell, relative to a control.
10. The method of embodiment 9, wherein the effective amount is sufficient to decrease the proteasomal activity of the cell by at least 25%, at least 30%, at least 40%, at least 45%, or at least 50%.
11. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
12. The method of embodiment 11, wherein the effective amount is sufficient to induce an average cellular rejuvenation of at least 25 years.
13. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
14. The method of embodiment 13, wherein the cells are fibroblasts.
15. The method of embodiment 14, wherein the fibroblasts are human dermal fibroblasts.
16. The method of any one of the preceding embodiments, wherein the nucleic acid comprises a heterologous promoter operably linked to the open reading frame.
17. The method of embodiment 16, wherein the heterologous promoter is an inducible promoter.
18. The method of any one of the preceding embodiments, comprising delivering to cells the inhibitor of ESR1 protein expression.
19. The method of any one of the preceding embodiments, comprising delivering to cells the inhibitor of expression of the nucleic acid that encodes the ESR1 protein.
20. The method of any one of the preceding embodiments, wherein the inhibitor is delivered on a non-viral vector or a viral vector.
21. The method of any one of the preceding embodiments, wherein the contacting comprises transfecting the cells.
22. The method of any one of the preceding embodiments, wherein the method comprises reducing expression or activity of endogenous ESR1 protein at a level that is lower than a baseline level.

### Additional Embodiments Relating to MAPK7

1. A method of inducing cellular rejuvenation of a cell comprising contacting the cell with an effective amount of an inhibitor of MAPK7 protein expression or expression of a nucleic acid encoding the MAPK7 protein.
2. A method of inducing cellular rejuvenation of a cell in a subject comprising administering to the subject an effective amount of an inhibitor of MAPK7 protein expression or expression of a nucleic acid encoding the MAPK7 protein.
3. The method of embodiment 1 or 2, wherein the inhibitor is a CRSPR-Cas9 system, a CRISPRi system, a CRISPRoff system, an shRNA, an siRNA, or a small molecule inhibitor.
4. The method of any one of the preceding embodiments, wherein the effective amount is sufficient to decrease cellular senescence of the cell, relative to a control.
5. The method of embodiment 4, wherein the effective amount is sufficient to decrease cellular senescence of the cell by at least 20%, at least 30%, at least 40%, or at least 50%.
6. The method of embodiment 5, wherein the effective amount is sufficient to decrease cellular senescence of the cell by at least 50%.
7. The method of any one of embodiments 4-6, wherein the effective amount is sufficient to decrease senescence-associated β-galactosidase activity of the cell, relative to a control.
8. The method of embodiment 7, wherein the effective amount is sufficient to decrease the senescence-associated β-galactosidase activity of the cell by at least 25%, at least 30%, t least 35%, at least 40%, at least 45%, or at least 50%.
9. The method of any one of embodiments 4-8, wherein the effective amount is sufficient to decrease proteasomal activity of the cell, relative to a control.
10. The method of embodiment 9, wherein the effective amount is sufficient to decrease the proteasomal activity of the cell by at least 25%, at least 30%, at least 40%, at least 45%, or at least 50%.
11. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
12. The method of embodiment 11, wherein the effective amount is sufficient to induce an average cellular rejuvenation of at least 25 years.
13. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
14. The method of embodiment 13, wherein the cells are fibroblasts.
15. The method of embodiment 14, wherein the fibroblasts are human dermal fibroblasts.
16. The method of any one of the preceding embodiments, wherein the nucleic acid comprises a heterologous promoter operably linked to the open reading frame.
17. The method of embodiment 16, wherein the heterologous promoter is an inducible promoter.
18. The method of any one of the preceding embodiments, comprising delivering to cells the inhibitor of MAPK7 protein expression.
19. The method of any one of the preceding embodiments, comprising delivering to cells the inhibitor of expression of the nucleic acid that encodes the MAPK7 protein.
20. The method of any one of the preceding embodiments, wherein the inhibitor is delivered on a non-viral vector or a viral vector.
21. The method of any one of the preceding embodiments, wherein the contacting comprises transfecting the cells.
22. The method of any one of the preceding embodiments, wherein the method comprises reducing expression or activity of endogenous MAPK7 protein at a level that is lower than a baseline level.

### Additional Embodiments Relating to KDM6A

1. A method of inducing cellular rejuvenation of a cell comprising contacting the cell with an effective amount of an inhibitor of KDM6A protein expression or expression of a nucleic acid encoding the KDM6A protein.
2. A method of inducing cellular rejuvenation of a cell in a subject comprising administering to the subject an effective amount of an inhibitor of KDM6A protein expression or expression of a nucleic acid encoding the KDM6A protein.
3. The method of embodiment 1 or 2, wherein the inhibitor is a CRSPR-Cas9 system, a CRISPRi system, a CRISPRoff system, an shRNA, an siRNA, or a small molecule inhibitor.
4. The method of any one of the preceding embodiments, wherein the effective amount is sufficient to decrease cellular senescence of the cell, relative to a control.
5. The method of embodiment 4, wherein the effective amount is sufficient to decrease cellular senescence of the cell by at least 20%, at least 30%, at least 40%, or at least 50%.
6. The method of embodiment 5, wherein the effective amount is sufficient to decrease cellular senescence of the cell by at least 50%.
7. The method of any one of embodiments 4-6, wherein the effective amount is sufficient to decrease senescence-associated-galactosidase activity of the cell, relative to a control.
8. The method of embodiment 7, wherein the effective amount is sufficient to decrease the senescence-associated β-galactosidase activity of the cell by at least 25%, at least 30%, t least 35%, at least 40%, at least 45%, or at least 50%.
9. The method of any one of embodiments 4-8, wherein the effective amount is sufficient to decrease proteasomal activity of the cell, relative to a control.
10. The method of embodiment 9, wherein the effective amount is sufficient to decrease the proteasomal activity of the cell by at least 25%, at least 30%, at least 40%, at least 45%, or at least 50%.
11. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
12. The method of embodiment 11, wherein the effective amount is sufficient to induce an average cellular rejuvenation of at least 25 years.
13. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
14. The method of embodiment 13, wherein the cells are fibroblasts.
15. The method of embodiment 14, wherein the fibroblasts are human dermal fibroblasts.
16. The method of any one of the preceding embodiments, wherein the nucleic acid comprises a heterologous promoter operably linked to the open reading frame.
17. The method of embodiment 16, wherein the heterologous promoter is an inducible promoter.
18. The method of any one of the preceding embodiments, comprising delivering to cells the inhibitor of KDM6A protein expression.
19. The method of any one of the preceding embodiments, comprising delivering to cells the inhibitor of expression of the nucleic acid that encodes the KDM6A protein.
20. The method of any one of the preceding embodiments, wherein the inhibitor is delivered on a non-viral vector or a viral vector.
21. The method of any one of the preceding embodiments, wherein the contacting comprises transfecting the cells.
22. The method of any one of the preceding embodiments, wherein the method comprises reducing expression or activity of endogenous KDM6A protein at a level that is lower than a baseline level.

### Additional Embodiments Relating to CTNNB1

1. A method of inducing cellular rejuvenation of a cell comprising contacting the cell with an effective amount of an inhibitor of CTNNB1 protein expression or expression of a nucleic acid encoding the CTNNB1 protein.
2. A method of inducing cellular rejuvenation of a cell in a subject comprising administering to the subject an effective amount of an inhibitor of CTNNB1 protein expression or expression of a nucleic acid encoding the CTNNB1 protein.
3. The method of embodiment 1 or 2, wherein the inhibitor is a CRSPR-Cas9 system, a CRISPRi system, a CRISPRoff system, an shRNA, an siRNA, or a small molecule inhibitor.
4. The method of any one of the preceding embodiments, wherein the effective amount is sufficient to decrease cellular senescence of the cell, relative to a control.
5. The method of embodiment 4, wherein the effective amount is sufficient to decrease cellular senescence of the cell by at least 20%, at least 30%, at least 40%, or at least 50%.
6. The method of embodiment 5, wherein the effective amount is sufficient to decrease cellular senescence of the cell by at least 50%.
7. The method of any one of embodiments 4-6, wherein the effective amount is sufficient to decrease senescence-associated β-galactosidase activity of the cell, relative to a control.
8. The method of embodiment 7, wherein the effective amount is sufficient to decrease the senescence-associated β-galactosidase activity of the cell by at least 25%, at least 30%, t least 35%, at least 40%, at least 45%, or at least 50%.
9. The method of any one of embodiments 4-8, wherein the effective amount is sufficient to decrease proteasomal activity of the cell, relative to a control.
10. The method of embodiment 9, wherein the effective amount is sufficient to decrease the proteasomal activity of the cell by at least 25%, at least 30%, at least 40%, at least 45%, or at least 50%.
11. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
12. The method of embodiment 11, wherein the effective amount is sufficient to induce an average cellular rejuvenation of at least 25 years.
13. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
14. The method of embodiment 13, wherein the cells are fibroblasts.
15. The method of embodiment 14, wherein the fibroblasts are human dermal fibroblasts.
16. The method of any one of the preceding embodiments, wherein the nucleic acid comprises a heterologous promoter operably linked to the open reading frame.
17. The method of embodiment 16, wherein the heterologous promoter is an inducible promoter.
18. The method of any one of the preceding embodiments, comprising delivering to cells the inhibitor of CTNNB1 protein expression.
19. The method of any one of the preceding embodiments, comprising delivering to cells the inhibitor of expression of the nucleic acid that encodes the CTNNB1 protein.
20. The method of any one of the preceding embodiments, wherein the inhibitor is delivered on a non-viral vector or a viral vector.
21. The method of any one of the preceding embodiments, wherein the contacting comprises transfecting the cells.
22. The method of any one of the preceding embodiments, wherein the method comprises reducing expression or activity of endogenous CTNNB1 protein at a level that is lower than a baseline level.

### Additional Embodiments Relating to Pro-Aging Proteins

1. A method of inducing cellular rejuvenation of a cell comprising contacting the cell with an effective amount of an inhibitor of expression of one or more pro-aging proteins or expression of one or more nucleic acids encoding the one or more pro-aging proteins.
2. A method of inducing cellular rejuvenation of a cell in a subject comprising administering to the subject an effective amount of an inhibitor of expression of one or more pro-aging proteins or expression of one or more nucleic acids encoding the one or more pro-aging proteins.
3. The method of embodiment 1 or 2, wherein the one or more pro-aging proteins are selected from the group consisting of: KAT7, ESR1, MAPK7, KDM6A, and CTNNB1.
4. The method of any one of the preceding embodiments, wherein the inhibitor is a CRSPR-Cas9 system, a CRISPRi system, a CRISPRoff system, an shRNA, an siRNA, or a small molecule inhibitor.
5. The method of any one of the preceding embodiments, wherein the effective amount is sufficient to decrease cellular senescence of the cell, relative to a control.
6. The method of embodiment 5, wherein the effective amount is sufficient to decrease cellular senescence of the cell by at least 20%, at least 30%, at least 40%, or at least 50%.
7. The method of embodiment 6, wherein the effective amount is sufficient to decrease cellular senescence of the cell by at least 50%.
8. The method of any one of embodiments 5-7, wherein the effective amount is sufficient to decrease senescence-associated β-galactosidase activity of the cell, relative to a control.
9. The method of embodiment 8, wherein the effective amount is sufficient to decrease the senescence-associated β-galactosidase activity of the cell by at least 25%, at least 30%, t least 35%, at least 40%, at least 45%, or at least 50%.
10. The method of any one of embodiments 5-9, wherein the effective amount is sufficient to decrease proteasomal activity of the cell, relative to a control.
11. The method of embodiment 10, wherein the effective amount is sufficient to decrease the proteasomal activity of the cell by at least 25%, at least 30%, at least 40%, at least 45%, or at least 50%.
12. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
13. The method of embodiment 12, wherein the effective amount is sufficient to induce an average cellular rejuvenation of at least 25 years.
14. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
15. The method of embodiment 14, wherein the cells are fibroblasts.
16. The method of embodiment 15, wherein the fibroblasts are human dermal fibroblasts.
17. The method of any one of the preceding embodiments, wherein the one or more nucleic acids comprises one or more heterologous promoters operably linked to one or more open reading frames.
18. The method of embodiment 17, wherein the one or more heterologous promoters are inducible promoters.
19. The method of any one of the preceding embodiments, comprising delivering to cells the inhibitor of expression of the one or more pro-aging proteins.
20. The method of any one of the preceding embodiments, comprising delivering to cells the inhibitor of expression of the one or more nucleic acids that encode the one or more pro-aging proteins.
21. The method of any one of the preceding embodiments, wherein the inhibitor is delivered on a non-viral vector or a viral vector.
22. The method of any one of the preceding embodiments, wherein the contacting comprises transfecting the cells.
23. The method of any one of the preceding embodiments, wherein the method comprises reducing expression or activity of endogenous one or more pro-aging proteins at a level that is lower than a baseline level.

### Additional Embodiments Relating to Anti-Aging and Pro-Aging Proteins

1. A method of inducing cellular rejuvenation of a cell comprising:
(i) contacting the cell with an effective amount of one or more anti-aging proteins or one or more nucleic acids encoding the one or more anti-aging proteins; and/or
(ii) contacting the cell with an effective amount of an inhibitor of expression of one or more pro-aging proteins or expression of one or more nucleic acids encoding the one or more pro-aging proteins.
2. A method of inducing cellular rejuvenation of a cell in a subject comprising:
(i) administering to the subject an effective amount of one or more anti-aging proteins or one or more nucleic acids encoding the one or more anti-aging proteins; and/or
   (ii) administering to the subject an effective amount of an inhibitor of expression of one or more pro-aging proteins or expression of one or more nucleic acids encoding the one or more pro-aging proteins.
3. The method of embodiment 1 or 2, wherein the one or more anti-aging proteins is selected from the group consisting of: SRSF1, SLC2A13, RNASEL, WDTC1, and NPM1.
4. The method of embodiment 1 or 2, wherein the one or more pro-aging proteins are selected from the group consisting of: KAT7, ESR1, MAPK7, KDM6A, and CTNNB1.
5. The method of any one of the preceding embodiments, wherein the inhibitor is a CRSPR-Cas9 system, a CRISPRi system, a CRISPRoff system, an shRNA, an siRNA, or a small molecule inhibitor.
6. The method of any one of the preceding embodiments, wherein the effective amount sufficient to decrease cellular senescence of the cell, relative to a control.

7. The method of embodiment 6, wherein the effective amount is sufficient to decrease cellular senescence of the cell by at least 20%, at least 30%, at least 40%, or at least 50%.
8. The method of embodiment 7, wherein the effective amount is sufficient to decrease cellular senescence of the cell by at least 50%.
9. The method of any one of embodiments 6-8, wherein the effective amount is sufficient to decrease senescence-associated β-galactosidase activity of the cell, relative to a control.
10. The method of embodiment 9, wherein the effective amount is sufficient to decrease the senescence-associated β-galactosidase activity of the cell by at least 25%, at least 30%, t least 35%, at least 40%, at least 45%, or at least 50%.
11. The method of any one of embodiments 6-10, wherein the effective amount is sufficient to decrease proteasomal activity of the cell, relative to a control.
12. The method of embodiment 11, wherein the effective amount is sufficient to decrease the proteasomal activity of the cell by at least 25%, at least 30%, at least 40%, at least 45%, or at least 50%.
13. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
14. The method of embodiment 13, wherein the effective amount is sufficient to induce an average cellular rejuvenation of at least 25 years.
15. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
16. The method of embodiment 15, wherein the cells are fibroblasts.
17. The method of embodiment 16, wherein the fibroblasts are human dermal fibroblasts.
18. The method of any one of the preceding embodiments, wherein the one or more nucleic acids comprise one or more heterologous promoters operably linked to one or more open reading frames.
19. The method of embodiment 18, wherein the one or more heterologous promoters are inducible promoters.
20. The method of any one of the preceding embodiments, comprising delivering to cells the one or more anti-aging proteins.
21. The method of any one of the preceding embodiments, comprising delivering to cells the inhibitor of expression of the one or more pro-aging proteins.
22. The method of any one of the preceding embodiments, comprising delivering to cells the one or more nucleic acids comprising one or more open reading frames encoding the one or more anti-aging proteins.
23. The method of any one of the preceding embodiments, comprising delivering to cells the inhibitor of expression of the one or more nucleic acids that encode the one or more pro-aging proteins.
24. The method of any one of the preceding embodiments, wherein the one or more nucleic acids is delivered on a non-viral vector or a viral vector.
25. The method of any one of the preceding embodiments, wherein the contacting comprises transfecting the cells.
26. A method of inducing cellular rejuvenation of a cell comprising overexpressing in the cell an effective amount of one or more anti-aging proteins.
27. The method of embodiment 26, wherein the method comprises activating expression or activity of the endogenous one or more anti-aging proteins at a level that is higher than a baseline level.
28. The method of any one of the preceding embodiments, wherein the method comprises reducing expression or activity of endogenous one or more pro-aging proteins at a level that is lower than a baseline level.

### Additional Embodiments

1. A method of inducing cellular rejuvenation of a cell comprising contacting the cell with an effective amount of a protein of Table 3 or a nucleic acid encoding the protein of Table 3.
2. A method of inducing cellular rejuvenation of a cell in a subject comprising administering to the subject an effective amount of a protein of Table 3 or a nucleic acid encoding the protein of Table 3.
3. The method of embodiment 1 or 2, wherein the effective amount is sufficient to decrease cellular senescence of the cell, relative to a control.
4. The method of embodiment 3, wherein the effective amount is sufficient to decrease cellular senescence of the cell by at least 20%, at least 30%, at least 40%, or at least 50%.
5. The method of embodiment 4, wherein the effective amount is sufficient to decrease cellular senescence of the cell by at least 50%.
6. The method of any one of embodiments 3-5, wherein the effective amount is sufficient to decrease senescence-associated β-galactosidase activity of the cell, relative to a control.
7. The method of embodiment 6, wherein the effective amount is sufficient to decrease the senescence-associated β-galactosidase activity of the cell by at least 25%, at least 30%, t least 35%, at least 40%, at least 45%, or at least 50%.
8. The method of any one of embodiments 3-7, wherein the effective amount is sufficient to decrease proteasomal activity of the cell, relative to a control.
9. The method of embodiment 8, wherein the effective amount is sufficient to decrease the proteasomal activity of the cell by at least 25%, at least 30%, at least 40%, at least 45%, or at least 50%.
10. The method of any one of the preceding embodiments, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
11. The method of embodiment 10, wherein the effective amount is sufficient to induce an average cellular rejuvenation of at least 25 years.
12. The method of any one of the preceding embodiments, wherein the cells are selected from fibroblasts, hematopoietic stem cells, endothelial cells, chondrocytes, skeletal muscle stem cells, keratinocytes, mesenchymal stem cells and corneal epithelial cells.
13. The method of embodiment 12, wherein the cells are fibroblasts.
14. The method of embodiment 13, wherein the fibroblasts are human dermal fibroblasts.
15. The method of any one of the preceding embodiments, wherein the nucleic acid comprises a heterologous promoter operably linked to the open reading frame.
16. The method of embodiment 15, wherein the heterologous promoter is an inducible promoter.
17. The method of any one of the preceding embodiments, comprising delivering to cells the protein of Table 3.
18. The method of any one of the preceding embodiments, comprising delivering to cells the nucleic acid comprising an open reading frame encoding the protein of Table 3.
19. The method of any one of the preceding embodiments, wherein the nucleic acid is delivered on a non-viral vector or a viral vector.
20. The method of any one of the preceding embodiments, wherein the contacting comprises transfecting the cells.
21. A method of inducing cellular rejuvenation of a cell comprising overexpressing in the cell an effective amount of a protein of Table 3.
22. The method of embodiment 21, wherein the method comprises activating expression or activity of endogenous protein of Table 3 at a level that is higher than a baseline level.

### EXAMPLES

The transcriptome is a key determinant of the cell phenotype and regulates its identity and function. There is a growing body of evidence that supports a systems-level view of cell engineering, whereby exogenous signals in the form of gene perturbations can induce desired cell states. For example, overexpressing the four Yamanaka factors was shown to not only dedifferentiate a somatic cell to a pluripotent state, but also reverse the age-related functional decline in old cells, thereby supporting a view of aging as a transcriptomic state.

Throughout aging, there is an increased instability in gene expression, which serves as the basis for numerous aging clocks trained at either the epigenetic or transcriptomic level. However, little is known about the functional relevance of DNA methylation sites in epigenetic clocks⁵, and the current RNA clocks use difficult to interpret age classifiers or offer limited insight into the aging process. This lack of interpretability precludes the use of these clocks as proxies for cellular function in the study of aging.

The focus of this study was to explore transcriptomic reprogramming as an approach to cellular rejuvenations. ATranscriptomic Interpretable Multi-process Ensemble (TIME) predictor was first developed that can accurately measure the age of human fibroblasts and respond to known biological interventions. Then, the TIME predictor was used to perform a cDNA overexpression screen for identifying rejuvenation perturbations in aged human cells. Leveraging the functional interpretability of the clock, transcriptomic differences in the aging phenotype of cells from different old donors are described and the effect of the gene perturbations on key cellular processes in aging was analyzed. Lastly, SRSF1 was discovered as a novel age-modulating gene, whose overexpression reprogrammed the transcriptome to a more youthful state through the differential splicing of genes involved in histone methylation and translation initiation.

### Example 1

### Process-Based Transcriptomic Aging Clock

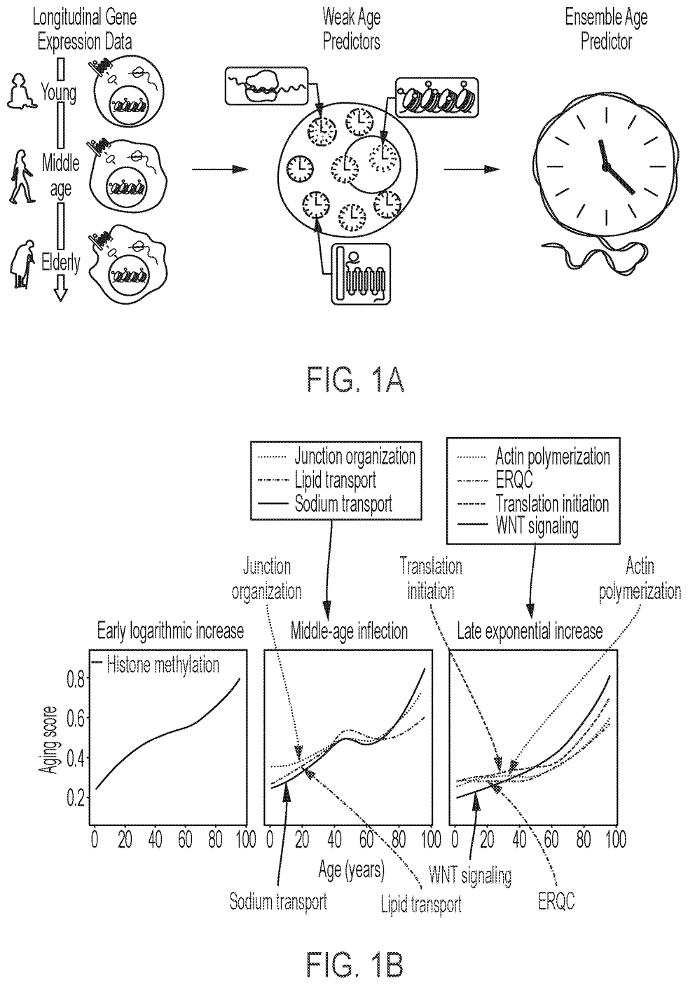

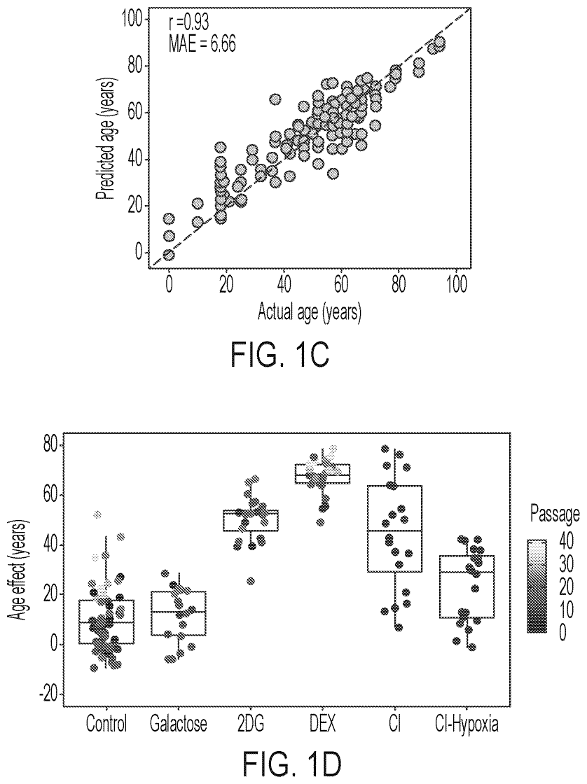

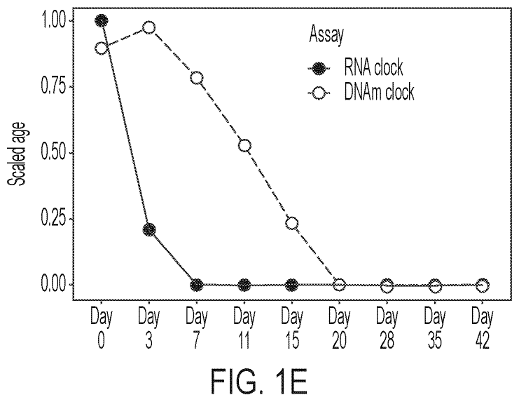

Using a published RNA-sequencing (RNA-Seq) dataset from primary normal human dermal fibroblasts (NHDF) aged 1-96 years old, along with sequencing data from similar fibroblasts, a machine learning predictor was trained on the chronological age of the sample donor. This approach included a layer of functional interpretability by sub-setting the transcriptome into cellular processes using the Molecular Biology of the Cell Ontology. To develop the clock, process-specific weak age predictors were trained using a generalized linear model (GLM) for identifying the cellular processes most predictive of age (FIG. 1A). This approach stably selected 8 processes (WNT signaling, Histone methylation, Junction organization, Translation initiation, Actin polymerization, Lipid transport, ER quality control (ERQC), and Sodium transport) with varying relative contributions to age prediction (data not shown). Furthermore, the expression levels for the genes in these 8 processes were used to train an ensemble predictor, which achieved accurate age predictions using only 146 genes (Table 1 and data not shown). Finally, aging was observed in a process-specific manner across the human lifespan and identified three distinct trajectories (FIG. 1B): early logarithmic increase (Histone methylation), middle-age inflection (Junction organization, Lipid transport, Sodium transport), and late exponential increase (Translation initiation, Actin polymerization, ERQC, WNT signaling).

The TIME predictor was applied to 12 other different datasets and observed a similar performance to that of widely-used epigenetic clocks (Pearson's r=0.93, p-value <2.2e-16; MAE=6.66)⁵, showcasing the accuracy and robustness of the RNA clock (FIG. 1C). Moreover, the TIME predictor showed good agreement with the epigenetic clock in measuring the in vitro aging of skin fibroblasts (1 year≅0.7 PD), but was not as accurate when applied to lung fibroblast, implying tissue specificity (data not shown). When applying the clock to cells exposed to age-modulating treatments, expected age-related changes were observed in the transcriptomes of progeroid samples, as well as cells exposed to Rapamycin, hypoxia, and various stresses (FIG. 1D).

Using the clock on a time course dataset of fibroblast reprogramming, a drastic decrease in the predicted age was observed after only 3 days (FIG. 1E). In comparison, the widely used DNA methylation (DNAm) clock achieved similar age predictions only at day 15 of reprogramming, after which the two assays reached a consensus at the iPSC stage. This suggests a shorter response time for the transcriptomic clock, which is in line with the measured effects of Rapamycin in young and middle-aged cells (data not shown). We believe The observed accelerated transcriptional rejuvenation were believed to be due to the dynamic nature of gene expression networks as compared to the more stable and slower-changing DNAm patterns. When analyzing process-specific activity across the reprogramming timeline, early rejuvenation was discovered to be driven by changes in genes part of WNT signaling, histone methylation, and lipid transport, which acquire a stable young state after 7 days (data not shown).

### Age Reversal Screen Identifies Genes for Cellular Rejuvenation

Next, the TIME predictor was used to identify novel rejuvenating interventions by performing a cDNA overexpression screen in human cells. Using the published target identification method (Kramme, C. et al. Cell Reports Methods (2021)), a publicly available NHDF RNA-Seq dataset was analyzed and generated a ranked list of genes predicted to be highly influential in the aging process (Table 2). 89 genes were chosen to test for their effect on the aging phenotype and included KAT7 (a gene implicated in fibroblast senescence) as well as the Yamanaka factors in a polycistronic cassette (OSKM), as positive controls.

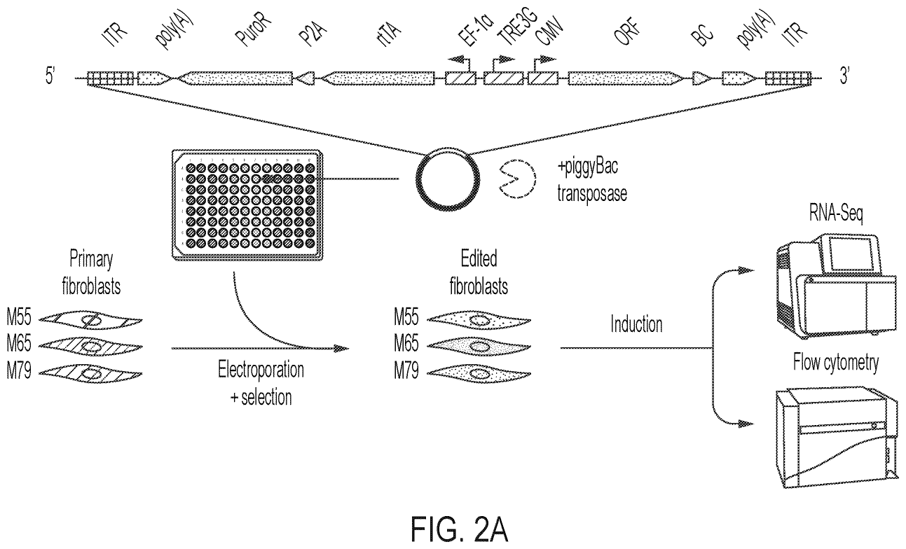

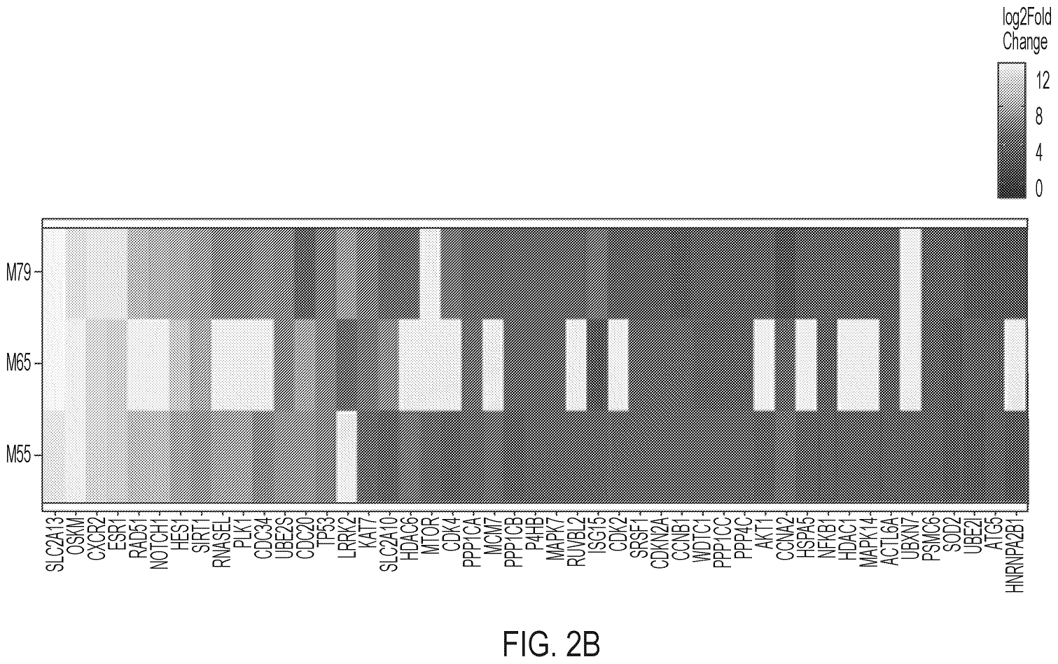

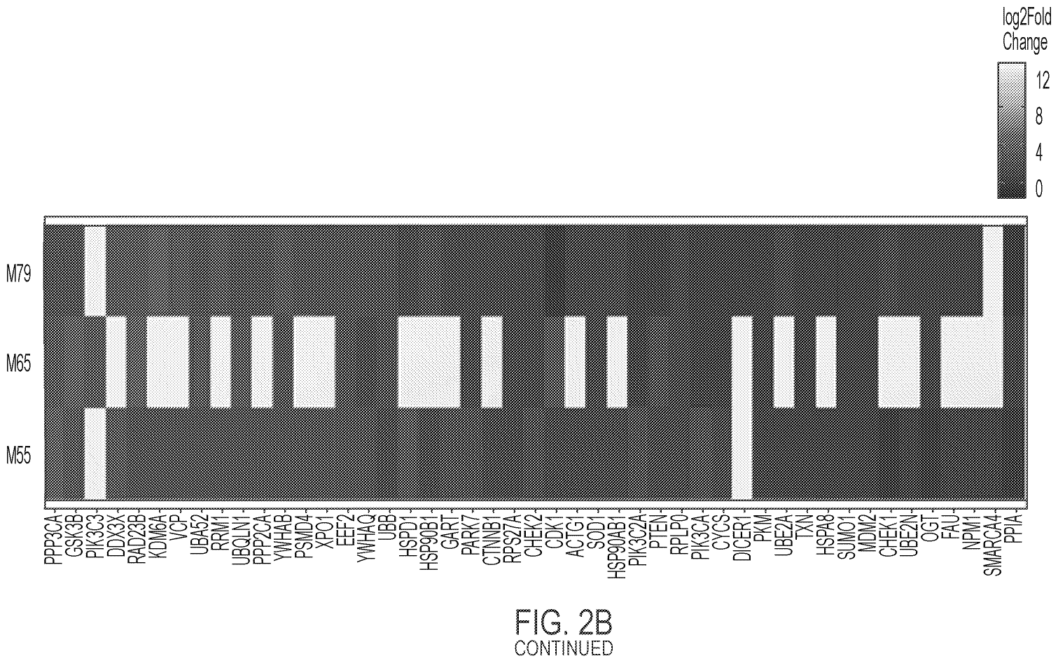

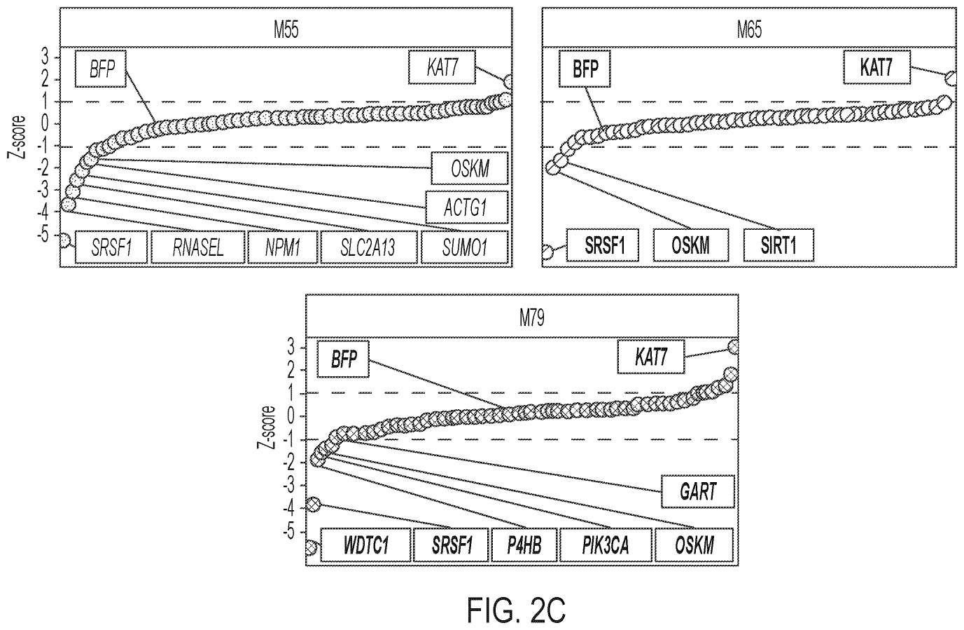

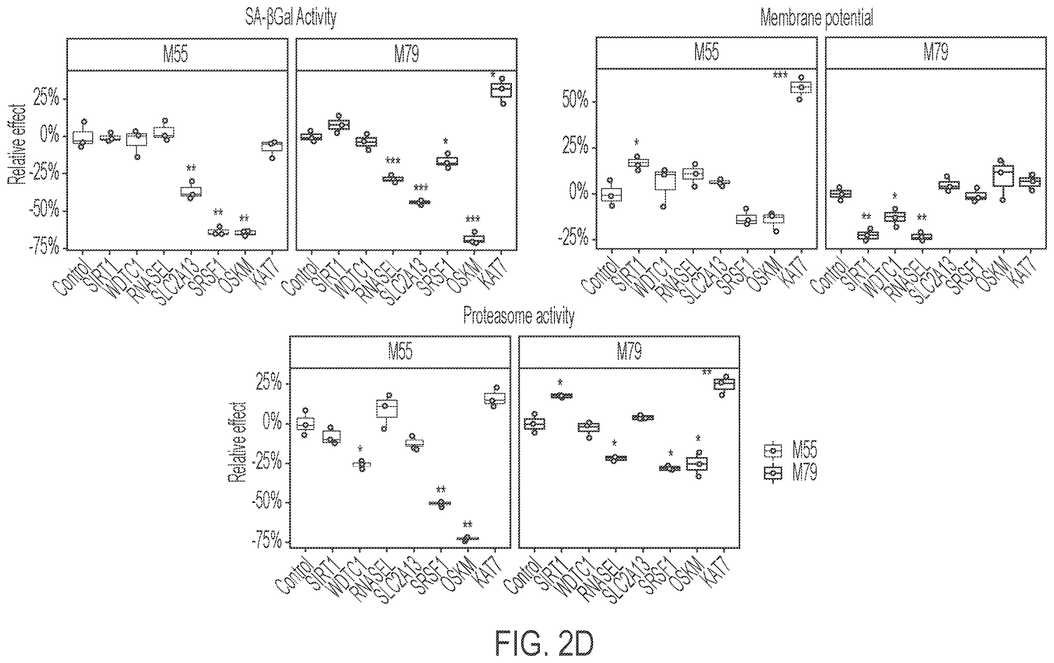

To test the potential age-modulating effect of the target genes, individual overexpression lines of the candidates in 3 different primary NHDF lines from 55-, 65-, and 79-year-old donors were generated. After selecting for integrants, the expression of the transgenes were induced and age-related changes were assayed using RNA-Seq and flow cytometry-based assays (FIG. 2A). A wide range of gene induction levels among the 95 genes were observed, ranging from no overexpression to a 4000-fold increase (FIG. 2B). However, there seemed to be good agreement in the overexpression levels between the 3 NHDF lines, indicating that induction differences are gene specific. The expression of most of the transgenes and the identity of the lines were confirmed by performing a barcode search on the raw sequencing reads (data not shown). The results agreed with the fold change data, and it was discovered that the basal endogenous gene expression level is negatively correlated with the observed overexpression levels (data not shown), confirming previous findings that there are gene-specific induction limits.

The effect of the genetic perturbations on the aging phenotype of the three NHDF lines was analyzed using the TIME predictor. The clock correctly estimated the ages of the WT NHDF lines (Table 3) and was able to detect age-modulating effects of the controls (FIG. 2C). OSKM had, as expected, a strong rejuvenating effect across all three lines, while KAT7 had the highest pro-aging effect. Most notably, several genes were discovered that induced significant changes towards a young transcriptome (−20 to −50 years) across all 3 NHDF lines (FIG. 2C), including SIRT1, SLC2A13, SRSF1, RNASEL, and WDTC1. While the role of SIRT1 in aging has been thoroughly studied, the other four hits have limited information available about their role in aging. SLC2A13 was found to decrease during aging in the human dorsolateral prefrontal cortex and was recently identified as a risk gene for Parkinson's disease. The expression of SRSF1 is significantly reduced in old age and associated with parental longevity in humans. RNASEL levels in human serum were shown to be inversely correlated with metabolic syndrome and age. While WDTC1 has not been previously linked to aging, it was associated with lower fat mass and heightened insulin sensitivity in humans.

For an orthogonal readout of aging, three staining assays that report on important cellular features were conducted: senescence associated β-galactosidase activity (SA-βGal activity), mitochondrial membrane potential, and proteasomal activity. The data showed that OSKM, RNASEL, SLC2A13, and SRSF1 reduced the senescence phenotype in both M55 and M79 (FIG. 2D). In the mitochondrial assay, opposing effects in the two lines were observed, with SIRT1 increasing the mitochondrial potential of M55 and decreasing that of M79 (FIG. 2D). SRSF1 and OSKM both decreased the potential in M55, but increased it in M79, although not reaching the significance threshold. This, along with prior observations of large variability in mitochondrial membrane potential across different donors, suggests a potential homeostasis point. Lastly, the proteasome assay showed a decrease in activity in the OSKM and SRSF1 overexpression lines in both M55 and M79 (FIG. 2D). This reduction in 26S proteasome activity was unexpected, but, surprisingly, the known pro-aging gene KAT7 increased proteasomal activity in the assay. It was hypothesized that rejuvenating interventions may lead to increased proteostasis, thereby lowering the burden on the proteasome and reducing its expression and measured activity. Future studies aimed at measuring proteotoxicity would be necessary to better explain this result. Finally, the observed differential responses between the NHDF lines in both the transcriptomic and functional assays (data not shown) suggest that donor cell line-intrinsic differences in the starting transcriptome might influence the outcome of the perturbations.

### Transcriptomic Variability in Aging Phenotypes

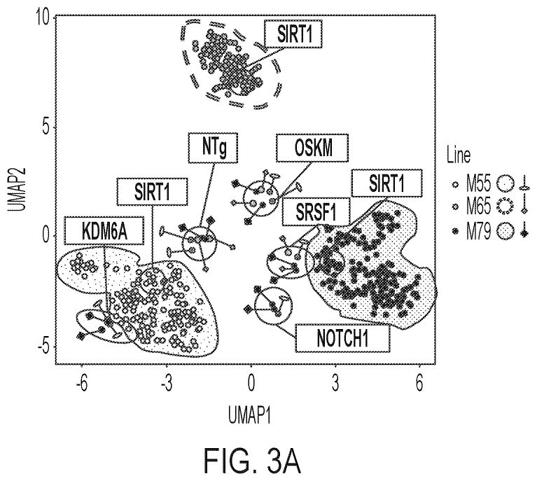

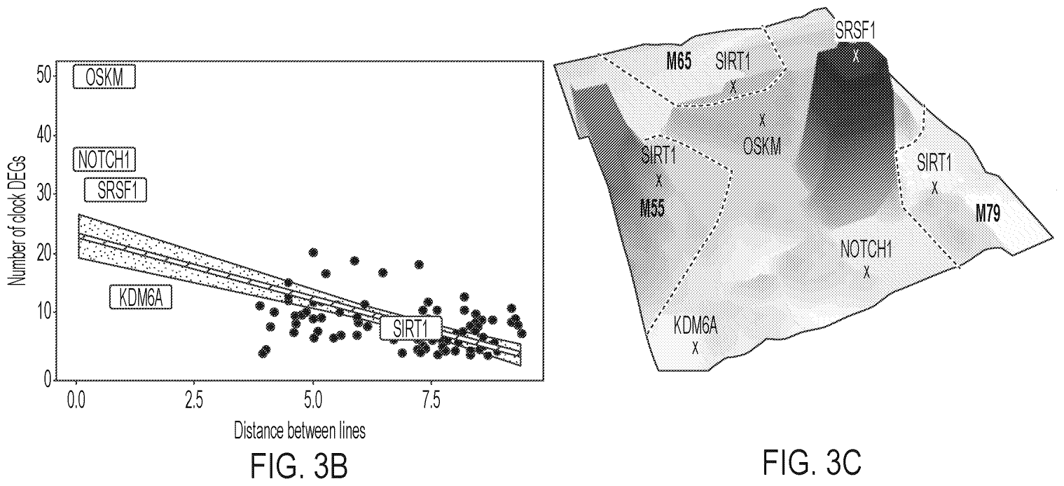

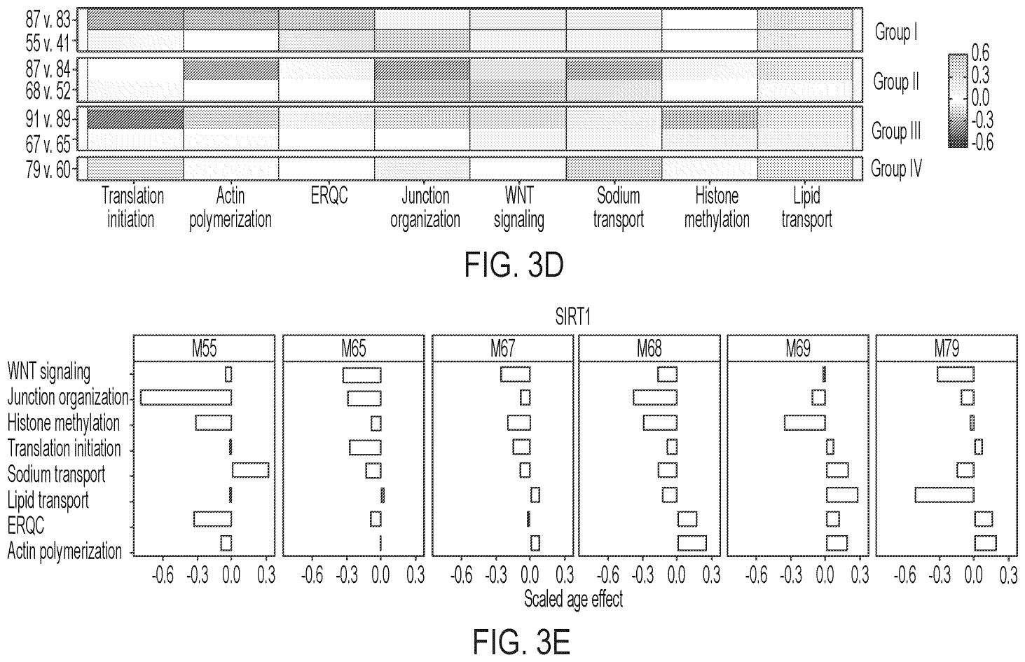

To further study the differences in the aging transcriptome between the three initial cell lines, a Uniform Manifold Approximation and Projection (UMAP) analysis was conducted and distinct clusters corresponding to the three NHDF donors were observed (FIG. 3A). This suggests that following the stress of nucleofection and the selection process, the three cell lines expanded their transcriptomic differences, which are more pronounced than gene expression changes induced by the perturbations. Differences in the WT aging phenotypes were then analyzed through the perspective of the clock processes by focusing on isogenic samples from individuals at different ages across their lifespan (2-19 years age difference). When quantifying the activity of the eight clock processes, it was noticed that NHDFs fall into four distinct groups defined by different types of dysfunctions (FIG. 3D): Group I=ERQC and lipid transport, Group II=WNT signaling and sodium transport, Group III=histone methylation, and Group IV=translation initiation and sodium transport. These results contextualize the different responses of the same perturbation in multiple cell lines and showcase the variability of human aging, in line with a recent study that described four distinct human aging patterns based on the types of molecular patterns that change over time.

Interestingly, while most of the gene inductions showed different age effects across the three lines (Table 3), it was discovered that some of the perturbations clustered together in UMAP space (FIG. 3A), overcoming line-specific differences. It was hypothesized that these genes (KDM6A, NOTCH1, OSKM, and SRSF1) induced robust transcriptomic reprogramming due to their central role in regulating gene expression, which overcame differences in the starting gene regulatory networks. In agreement with this hypothesis, a strong negative correlation was observed (Pearson's r=−0.64, p-value=1.678e-11) between the number of clock genes affected by a perturbation and the mean distance in UMAP space across overexpression lines for the same gene (FIG. 3B). Moreover, similar to the Waddington landscape analogy, a transcriptomic landscape of fibroblast aging was generated by embedding the predicted age of the samples to the UMAP plot and noticed that SRSF1 and OSKM had robust rejuvenating effects, while KDM6A and NOTCH1 had significant pro-aging effects (FIG. 3C). This suggests that perturbations that have a strong effect on the aging phenotype, as measured by the RNA clock, push the cells into a common transcriptional space, while the effect of weaker perturbations is dependent on the initial transcriptional state (data not shown).

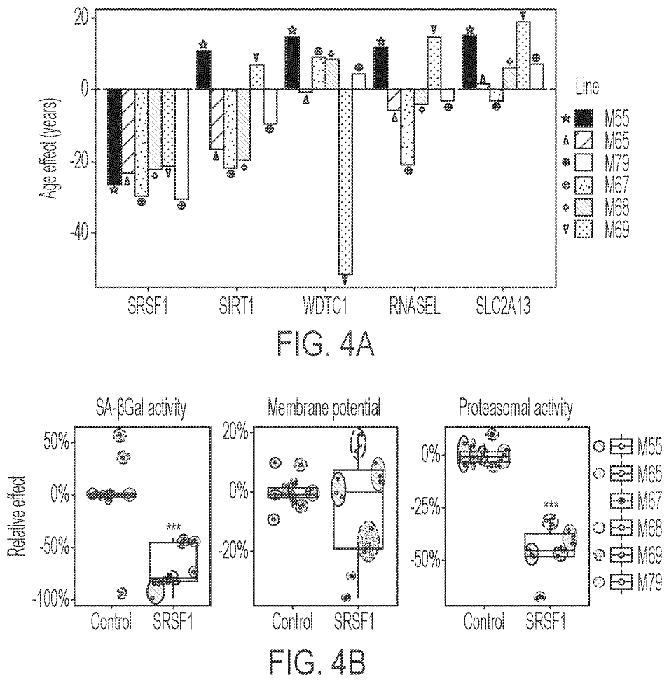

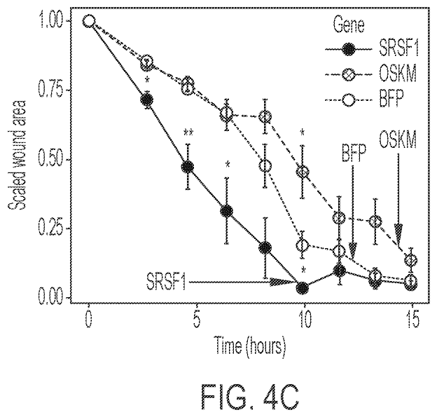

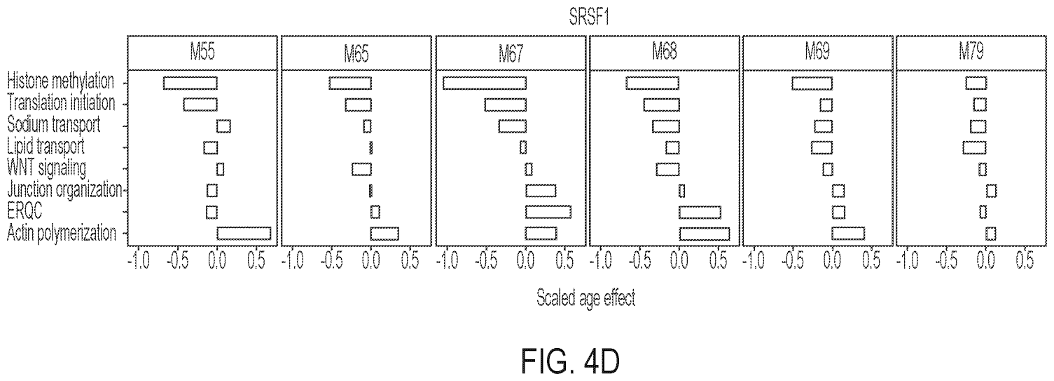

One example of a weak perturbation that has cell-line dependent effects is SIRT1 overexpression. Several reports have linked SIRT1 levels to lifespan extension in yeast, but not in worms, flies, or mice, thereby questioning its role in the aging process. In the initial screen, SIRT1 showed transcriptomic rejuvenation effects in two of the three lines, with only one being statistically significant. Similarly, when the gene overexpression was repeated in 6 different lines, a strong age reversal effect was observed in only half the lines (FIG. 4A), further confirming SIRT1 overexpression as a weak perturbation with cell-line specific effects. When looking at the scaled age effect of SIRT1 on each of the clock processes, no common pattern was noticed between all lines, which also suggests that the sirtuin overexpression has line-specific aging effects (FIG. 3E). Nevertheless, in the responder lines (M65, M67, M68), a slight improvement in WNT signaling was observed, suggesting that SIRT1 might act on the WNT pathway in these cells.

TABLE 1
Genes included in the RNA clock.
Genes included in the RNA clock are listed as Ensembl IDs (“Predictor” column) and gene symbols.
The raw and standardized contribution of each gene to the age prediction is provided in the “Coefficient”
and “Standardized Coefficient” column, respectively. In addition, the processes each gene is associated
with in the Molecular Biology of the Cell Ontology is shown (“Process” column).

| | Gene | | Standardized | |
|---|---|---|---|---|
| Predictor | Symbol | Coefficient | Coefficient | Process |
| Intercept | — | 3.676114809 | −0.547921048 | — |
| ENSG00000148737 | TCF7L2 | 0.203423062 | 0.145205231 | WNT signaling |
| ENSG00000165617 | DACT1 | −0.104577043 | −0.11158401 | WNT signaling |
| ENSG00000168036 | CTNNB1 | 1.155505293 | 0.241076129 | Junction organization; WNT signaling |
| ENSG00000134245 | WNT2B | −0.080958237 | −0.09063118 | WNT signaling |
| ENSG00000162552 | WNT4 | 0.065692601 | 0.146128577 | WNT signaling |
| ENSG00000184675 | AMER1 | −0.340425536 | −0.11510598 | WNT signaling |
| ENSG00000228630 | HOTAIR | −0.02660401 | −0.070668056 | Histone methylation; WNT signaling |
| ENSG00000106483 | SFRP4 | 0.054716313 | 0.1254804 | WNT signaling |
| ENSG00000131650 | KREMEN2 | −0.374212687 | −0.111098616 | WNT signaling |
| ENSG00000082701 | GSK3B | 0.165730251 | 0.048646235 | WNT signaling |
| ENSG00000163348 | PYGO2 | 0.602236784 | 0.12687599 | WNT signaling |
| ENSG00000164736 | SOX17 | 0.17694256 | 0.099658795 | WNT signaling |
| ENSG00000070018 | LRP6 | −0.400894674 | −0.145217517 | WNT signaling |
| ENSG00000004975 | DVL2 | −0.137932933 | −0.035690819 | WNT signaling |
| ENSG00000174804 | FZD4 | 0.04796222 | 0.05108055 | WNT signaling |
| ENSG00000134569 | LRP4 | 0.024747349 | 0.020981309 | WNT signaling |
| ENSG00000138795 | LEF1 | 0.084010082 | 0.109813402 | WNT signaling |
| ENSG00000108375 | RNF43 | 0.057468549 | 0.034638674 | WNT signaling |
| ENSG00000162337 | LRP5 | −0.064028332 | −0.061243317 | WNT signaling |
| ENSG00000154856 | APCDD1 | 0.013634361 | 0.027832253 | WNT signaling |
| ENSG00000183762 | KREMEN1 | −0.192852626 | −0.112828097 | WNT signaling |
| ENSG00000165879 | FRAT1 | 0.099806158 | 0.067259579 | WNT signaling |
| ENSG00000135925 | WNT10A | 0.138072373 | 0.037215726 | WNT signaling |
| ENSG00000188763 | FZD9 | −0.05298353 | −0.056740297 | WNT signaling |
| ENSG00000155011 | DKK2 | 0.038131278 | 0.084632705 | WNT signaling |
| ENSG00000114251 | WNT5A | −0.057537968 | −0.061460551 | WNT signaling |
| ENSG00000075290 | WNT8B | 0.389034126 | 0.140364369 | WNT signaling |
| ENSG00000157240 | FZD1 | −0.078188613 | −0.033630775 | WNT signaling |
| ENSG00000177283 | FZD8 | 0.050571533 | 0.054071337 | WNT signaling |
| ENSG00000007372 | PAX6 | −0.043215988 | −0.079755094 | WNT signaling |
| ENSG00000125249 | RAP2A | 0.16908097 | 0.060736505 | WNT signaling |
| ENSG00000122515 | ZMIZ2 | −0.038528623 | −0.01385182 | WNT signaling |
| ENSG00000126457 | PRMT1 | −0.227667683 | −0.087422616 | Histone methylation |
| ENSG00000115548 | KDM3A | −0.297243909 | −0.109263437 | Histone methylation |
| ENSG00000164256 | PRDM9 | 302.2036775 | 0.016097875 | Histone methylation |
| ENSG00000138336 | TET1 | −0.024934291 | −0.021778778 | Histone methylation |
| ENSG00000187605 | TET3 | 0.069005463 | 0.052496081 | Histone methylation |
| ENSG00000142453 | CARM1 | −0.302278901 | −0.089161122 | Histone methylation |
| ENSG00000136504 | KAT7 | 0.919204701 | 0.168482405 | Histone methylation |
| ENSG00000143499 | SMYD2 | 0.309824718 | 0.075025188 | Histone methylation |
| ENSG00000133895 | MEN1 | −0.1676316 | −0.045443562 | Histone methylation |
| ENSG00000078403 | MLLT10 | 0.087182019 | 0.024400756 | Histone methylation |
| ENSG00000127663 | KDM4B | 0.226919391 | 0.091026956 | Histone methylation |
| ENSG00000055609 | KMT2C | −0.081517358 | −0.027199734 | Histone methylation |
| ENSG00000145391 | SETD7 | 0.361775259 | 0.117057071 | Histone methylation |
| ENSG00000107077 | KDM4C | 0.102464121 | 0.031609414 | Histone methylation |
| ENSG00000185787 | MORF4L1 | −0.150952912 | −0.03924061 | Histone methylation |
| ENSG00000116455 | WDR77 | 0.082572644 | 0.025394591 | Histone methylation |
| ENSG00000171681 | ATF7IP | 0.333836327 | 0.107711525 | Histone methylation |
| ENSG00000171988 | JMJDIC | −0.166670894 | −0.060685489 | Histone methylation |
| ENSG00000143379 | SETDB1 | −0.385548129 | −0.075841229 | Histone methylation |
| ENSG00000129691 | ASH2L | −0.364779419 | −0.075588272 | Histone methylation |
| ENSG00000176476 | SGF29 | 0.173076479 | 0.079762393 | Histone methylation |
| ENSG00000176658 | MYO1D | 0.118977805 | 0.115935697 | Junction organization |
| ENSG00000138771 | SHROOM3 | −0.03467626 | −0.025399217 | Junction organization |
| ENSG00000196405 | EVL | −0.321164897 | −0.142415479 | Actin polymerization; Junction |
| | | | | organization |
| ENSG00000123572 | NRK | −0.005980471 | −0.009225339 | Junction organization |
| ENSG00000123094 | RASSF8 | 0.119457967 | 0.054678427 | Junction organization |
| ENSG00000105245 | NUMBL | 0.216588615 | 0.075611974 | Junction organization |
| ENSG00000079112 | CDH17 | 0.009452929 | 0.004021506 | Junction organization |
| ENSG00000028203 | VEZT | −0.30110926 | −0.072680461 | Junction organization |
| ENSG00000107263 | RAPGEF1 | 0.24958251 | 0.05802994 | Junction organization |
| ENSG00000142279 | WTIP | −0.075224123 | −0.031969176 | Junction organization |
| ENSG00000066032 | CTNNA2 | −0.191004371 | −0.164717544 | Junction organization |
| ENSG00000128849 | CGNL1 | 0.133127066 | 0.160244025 | Junction organization |
| ENSG00000148498 | PARD3 | −0.68872521 | −0.168172831 | Junction organization |
| ENSG00000111052 | LIN7A | −0.055094728 | −0.064272703 | Junction organization |
| ENSG00000170558 | CDH2 | −0.081070801 | −0.084281445 | Junction organization |
| ENSG00000092820 | EZR | −0.0692457 | −0.060596153 | Junction organization |
| ENSG00000196581 | AJAP1 | 0.014087075 | 0.036484885 | Junction organization |
| ENSG00000105879 | CBLL1 | 0.24540869 | 0.07905453 | Junction organization |
| ENSG00000173801 | JUP | 0.03880525 | 0.03794143 | Junction organization |
| ENSG00000198910 | LICAM | −0.021540397 | −0.046639908 | Junction organization |
| ENSG00000163531 | NFASC | 0.148456805 | 0.086367754 | Junction organization |
| ENSG00000117155 | SSX2IP | −0.058873474 | −0.064761919 | Junction organization |
| ENSG00000156299 | TIAM1 | 0.018751852 | 0.026172225 | Junction organization |
| ENSG00000163412 | EIF4E3 | 0.05776898 | 0.053575212 | Translation initiation |
| ENSG00000148730 | EIF4EBP2 | 0.04712675 | 0.014552469 | Translation initiation |
| ENSG00000063046 | EIF4B | −0.2280096 | −0.086328 | Translation initiation |
| ENSG00000197111 | PCBP2 | 0.106929224 | 0.033134444 | Translation initiation |
| ENSG00000134030 | CTIF | 0.047308078 | 0.017456987 | Translation initiation |
| ENSG00000125977 | EIF2S2 | −0.22274827 | −0.065375001 | Translation initiation |
| ENSG00000158417 | EIF5B | 0.40563521 | 0.094520568 | Translation initiation |
| ENSG00000106682 | EIF4H | −0.372446843 | −0.093474236 | Translation initiation |
| ENSG00000132507 | EIF5A | 0.076915329 | 0.042019631 | Translation initiation |
| ENSG00000067248 | DHX29 | −0.252563915 | −0.062864723 | Translation initiation |
| ENSG00000178982 | EIF3K | 0.032302086 | 0.006318761 | Translation initiation |
| ENSG00000100353 | EIF3D | −0.042319294 | −0.010052054 | Translation initiation |
| ENSG00000110321 | EIF4G2 | −0.42109715 | −0.084644846 | Translation initiation |
| ENSG00000134001 | EIF2S1 | 0.601898328 | 0.125769833 | Translation initiation |
| ENSG00000130741 | EIF2S3 | −0.043741951 | −0.013654019 | Translation initiation |
| ENSG00000086232 | EIF2AK1 | 0.049218399 | 0.008409422 | Translation initiation |
| ENSG00000243056 | EIF4EBP3 | 0.140509067 | 0.133625323 | Translation initiation |
| ENSG00000034510 | TMSB10 | −0.258687809 | −0.090344164 | Actin polymerization |
| ENSG00000136754 | ABI1 | 0.703312755 | 0.161875393 | Actin polymerization |
| ENSG00000187239 | FNBP1 | −0.190728503 | −0.081926167 | Actin polymerization |
| ENSG00000090924 | PLEKHG2 | −0.330897417 | −0.120198563 | Actin polymerization |
| ENSG00000136732 | GYPC | 0.001727358 | 0.001148747 | Actin polymerization |
| ENSG00000106211 | HSPB1 | 0.043295352 | 0.037462604 | Actin polymerization |
| ENSG00000134215 | VAV3 | −0.039381555 | −0.058022034 | Actin polymerization |
| ENSG00000007237 | GAS7 | −0.02781765 | −0.032878578 | Actin polymerization |
| ENSG00000254999 | BRK1 | 0.302218194 | 0.067602702 | Actin polymerization |
| ENSG00000125753 | VASP | 0.015685437 | 0.007135417 | Actin polymerization |
| ENSG00000070087 | PFN2 | −0.144624282 | −0.071408446 | Actin polymerization |
| ENSG00000132970 | WASF3 | −0.225682275 | −0.145618229 | Actin polymerization |
| ENSG00000137478 | FCHSD2 | −0.057108071 | −0.020695706 | Actin polymerization |
| ENSG00000105443 | CYTH2 | −0.318138825 | −0.092354343 | Actin polymerization |
| ENSG00000197969 | VPS13A | 0.055511854 | 0.024584931 | Actin polymerization |
| ENSG00000100592 | DAAM1 | 0.057196414 | 0.030164331 | Actin polymerization |
| ENSG00000131504 | DIAPH1 | 0.090678132 | 0.02788895 | Actin polymerization |
| ENSG00000055163 | CYFIP2 | 0.099728834 | 0.119196271 | Actin polymerization |
| ENSG00000147676 | MAL2 | 0.093367114 | 0.054684027 | Actin polymerization |
| ENSG00000198848 | CES1 | 0.08904118 | 0.213807278 | Lipid transport |
| ENSG00000130208 | APOC1 | −0.069950992 | −0.073675416 | Lipid transport |
| ENSG00000087237 | CETP | −0.040538899 | −0.028076057 | Lipid transport |
| ENSG00000102595 | UGGT2 | −0.07181183 | −0.027930465 | ERQC |
| ENSG00000185825 | BCAP31 | 0.436033394 | 0.133785732 | ERQC |
| ENSG00000204308 | RNF5 | −0.303471855 | −0.08985336 | ERQC |
| ENSG00000116661 | FBXO2 | −0.129076237 | −0.137552948 | ERQC |
| ENSG00000239857 | GET4 | 0.174054233 | 0.11960688 | ERQC |
| ENSG00000155313 | USP25 | 0.089393503 | 0.037796398 | ERQC |
| ENSG00000147475 | ERLIN2 | −0.201119054 | −0.053173061 | ERQC |
| ENSG00000133872 | SARAF | 0.210988556 | 0.092044456 | ERQC |
| ENSG00000135506 | OS9 | −0.09068598 | −0.026793206 | ERQC |
| ENSG00000100350 | FOXRED2 | 0.252944926 | 0.122488326 | ERQC |
| ENSG00000184787 | UBE2G2 | 0.15662805 | 0.032885851 | ERQC |
| ENSG00000170881 | RNF139 | 0.140211116 | 0.061521906 | ERQC |
| ENSG00000113194 | FAF2 | 0.425512085 | 0.070863665 | ERQC |
| ENSG00000136731 | UGGT1 | −0.583578738 | −0.136072435 | ERQC |
| ENSG00000172071 | EIF2AK3 | 0.06871913 | 0.022151185 | ERQC; Translation initiation |
| ENSG00000115307 | AUP1 | −0.036892608 | −0.010667756 | ERQC |
| ENSG00000116406 | EDEM3 | 0.044310763 | 0.015588211 | ERQC |
| ENSG00000144285 | SCN1A | 0.056868128 | 0.127184712 | Sodium transport |
| ENSG00000110881 | ASIC1 | −0.039031864 | −0.021913201 | Sodium transport |
| ENSG00000136531 | SCN2A | 0.043863329 | 0.088218121 | Sodium transport |
| ENSG00000090020 | SLC9A1 | 0.260266387 | 0.07173017 | Sodium transport |
| ENSG00000138074 | SLC5A6 | −0.129596731 | −0.044213498 | Sodium transport |
| ENSG00000166828 | SCNN1G | 55.58189494 | 0.016097875 | Sodium transport |
| ENSG00000108684 | ASIC2 | 0.109669358 | 0.093913509 | Sodium transport |
| ENSG00000153253 | SCN3A | −0.094709183 | −0.121966637 | Sodium transport |
| ENSG00000010379 | SLC6A13 | −0.229526716 | −0.138485092 | Sodium transport |
| ENSG00000197818 | SLC9A8 | 0.077551666 | 0.019261143 | Sodium transport |
| ENSG00000072182 | ASIC4 | −0.0368954 | −0.030325705 | Sodium transport |
| ENSG00000111319 | SCNN1A | 0.121327298 | 0.117784901 | Sodium transport |
| ENSG00000158865 | SLC5A11 | −0.108365157 | −0.076438251 | Sodium transport |
| ENSG00000089327 | FXYD5 | 0.014910649 | 0.007213908 | Sodium transport |

TABLE 2
Candidate genes for age reversal screen.

| gene_symbol | DEG | ag_DEGscore | d_DEGscore | ag_rank | d_rank | ag_ns | d_ns | Borda |
|---|---|---|---|---|---|---|---|---|
| ACTG1 | DEG | 0.456 | 3.927 | 14 | 9 | 1.08E+05 | 5.94E+05 | 23 |
| ACTL6A | DEG | 0.529 | 0.934 | 19 | 43 | 1.06E+05 | 5.48E+05 | 62 |
| AKT1 | DEG | 0.985 | 3.813 | 90 | 49 | 9.42E+04 | 5.43E+05 | 139 |
| ATG5 | DEG | 0.056 | NA | 1026 | NA | 5.74E+04 | NA | NA |
| CCNA2 | DEG | 0.541 | 2.625 | 36 | 48 | 1.02E+05 | 5.44E+05 | 84 |
| CCNB1 | DEG | 1.296 | 4.689 | 21 | 30 | 1.05E+05 | 5.59E+05 | 51 |
| CDC20 | DEG | 0.697 | 5.481 | 57 | 132 | 9.77E+04 | 4.87E+05 | 189 |
| CDC34 | DEG | 0.115 | 2.258 | 65 | 130 | 9.71E+04 | 4.88E+05 | 195 |
| CDKI | DEG | 0.362 | 1.902 | 5 | 10 | 1.12E+05 | 5.94E+05 | 15 |
| CDK2 | DEG | 0.541 | 1.753 | 13 | 17 | 1.08E+05 | 5.77E+05 | 30 |
| CDK4 | DEG | 0.149 | 0.457 | 24 | 26 | 1.05E+05 | 5.65E+05 | 50 |
| CDKN2A | nonDEG | NA | NA | 17 | 8 | 1.07E+05 | 5.83E+05 | 25 |
| CHEK1 | DEG | 0.288 | 0.608 | 58 | 102 | 9.76E+04 | 4.99E+05 | 160 |
| CHEK2 | DEG | 0.529 | 0.847 | 75 | 89 | 9.55E+04 | 5.09E+05 | 164 |
| CTNNB1 | DEG | 0.743 | 0.886 | 49 | 37 | 9.89E+04 | 5.50E+05 | 86 |
| CXCR2 | nonDEG | NA | NA | 3030 | 2078 | 3.12E+04 | 1.80E+05 | 5108 |
| CYCS | nonDEG | NA | NA | 19 | 9 | 1.06E+05 | 5.80E+05 | 28 |
| DDX3X | DEG | 0.568 | 1.487 | 83 | 134 | 9.50E+04 | 4.86E+05 | 217 |
| DICER1 | DEG | 0.358 | 0.940 | 7 | 6 | 1.10E+05 | 6.02E+05 | 13 |
| EEF2 | DEG | 0.125 | 1.362 | 37 | 50 | 1.02E+05 | 5.42E+05 | 87 |
| ESR1 | DEG | 4.343 | 19.162 | 128 | 72 | 9.11E+04 | 5.21E+05 | 200 |
| FAU | DEG | 0.339 | 4.264 | 62 | 73 | 9.73E+04 | 5.21E+05 | 135 |
| GART | nonDEG | NA | NA | 18 | 26 | 1.06E+05 | 5.45E+05 | 44 |
| GSK3B | DEG | 0.609 | 6.045 | 39 | 29 | 1.00E+05 | 5.62E+05 | 68 |
| HDAC1 | DEG | 0.272 | 0.674 | 11 | 15 | 1.09E+05 | 5.80E+05 | 26 |
| HDAC6 | DEG | 0.459 | 1.075 | 72 | 81 | 9.55E+04 | 5.14E+05 | 153 |
| HES1 | nonDEG | NA | NA | 3271 | 2401 | 2.94E+04 | 1.63E+05 | 5672 |
| HNRNPA2B1 | DEG | 0.399 | 0.516 | 85 | 156 | 9.47E+04 | 4.79E+05 | 241 |
| HSP90AB1 | DEG | 0.143 | 0.453 | 9 | 13 | 1.10E+05 | 5.87E+05 | 22 |
| HSP90B1 | DEG | 0.769 | 0.388 | 43 | 53 | 9.95E+04 | 5.40E+05 | 96 |
| HSPA5 | DEG | 0.118 | 0.449 | 8 | 8 | 1.10E+05 | 5.97E+05 | 16 |
| HSPA8 | DEG | 0.199 | 0.656 | 1 | 1 | 1.21E+05 | 6.43E+05 | 2 |
| HSPD1 | DEG | 0.244 | 1.162 | 52 | 105 | 9.87E+04 | 4.97E+05 | 157 |
| ISG15 | DEG | 0.161 | 5.821 | 84 | 86 | 9.47E+04 | 5.10E+05 | 170 |
| KAT7 | nonDEG | NA | NA | 1477 | 1245 | 4.73E+04 | 2.41E+05 | 2722 |
| KDM6A | DEG | 0.059 | 0.329 | 4 | 11 | 1.12E+05 | 5.90E+05 | 15 |
| LRRK2 | DEG | 5.109 | 19.832 | 3 | 3 | 1.12E+05 | 6.23E+05 | 6 |
| MAPK14 | DEG | 0.235 | 1.305 | 34 | 22 | 1.02E+05 | 5.73E+05 | 56 |
| MAPK7 | DEG | 0.403 | 3.262 | 115 | 88 | 9.19E+04 | 5.09E+05 | 203 |
| MCM7 | DEG | 0.674 | 3.380 | 61 | 151 | 9.73E+04 | 4.80E+05 | 212 |
| MDM2 | DEG | 3.946 | 21.694 | 94 | 97 | 9.38E+04 | 5.03E+05 | 191 |
| MTOR | nonDEG | NA | NA | 9 | 2 | 1.09E+05 | 6.05E+05 | 11 |
| NFKB1 | DEG | 0.379 | 1.988 | 42 | 38 | 9.97E+04 | 5.50E+05 | 80 |
| NOTCH1 | DEG | 0.212 | 3.723 | 125 | 74 | 9.14E+04 | 5.20E+05 | 199 |
| NPM1 | DEG | 0.900 | 7.332 | 70 | 122 | 9.58E+04 | 4.90E+05 | 192 |
| OGT | DEG | 0.334 | 2.829 | 40 | 45 | 1.00E+05 | 5.47E+05 | 85 |
| P4HB | DEG | 0.259 | 0.618 | 116 | 112 | 9.18E+04 | 4.93E+05 | 228 |
| PARK7 | DEG | 0.132 | 0.469 | 80 | 92 | 9.50E+04 | 5.07E+05 | 172 |
| PIK3C2A | DEG | 0.187 | 0.606 | 118 | 119 | 9.17E+04 | 4.90E+05 | 237 |
| PIK3C3 | DEG | 0.281 | 0.349 | 126 | 110 | 9.12E+04 | 4.95E+05 | 236 |
| PIK3CA | DEG | 1.285 | 4.451 | 82 | 68 | 9.50E+04 | 5.24E+05 | 150 |
| PKM | DEG | 0.370 | 0.483 | 67 | 78 | 9.66E+04 | 5.15E+05 | 145 |
| PLK1 | DEG | 0.759 | 6.376 | 35 | 64 | 1.02E+05 | 5.29E+05 | 99 |
| PPIA | DEG | 0.780 | 2.225 | 117 | 109 | 9.18E+04 | 4.95E+05 | 226 |
| PPP1CA | DEG | 0.693 | 2.348 | 2 | 7 | 1.14E+05 | 5.97E+05 | 9 |
| PPP1CB | DEG | 2.996 | 14.670 | 17 | 25 | 1.07E+05 | 5.68E+05 | 42 |
| PPP1CC | nonDEG | NA | NA | 4 | 3 | 1.14E+05 | 5.99E+05 | 7 |
| PPP2CA | nonDEG | NA | NA | 5 | 10 | 1.11E+05 | 5.79E+05 | 15 |
| PPP3CA | DEG | 1.133 | 4.202 | 93 | 61 | 9.39E+04 | 5.30E+05 | 154 |
| PPP4C | DEG | 0.195 | 1.236 | 30 | 52 | 1.04E+05 | 5.41E+05 | 82 |
| PSMC6 | DEG | 0.141 | 0.353 | 51 | 87 | 9.87E+04 | 5.10E+05 | 138 |
| PSMD4 | DEG | 1.503 | 7.353 | 55 | 111 | 9.82E+04 | 4.93E+05 | 166 |
| PTEN | DEG | 0.956 | 4.870 | 45 | 34 | 9.94E+04 | 5.56E+05 | 79 |
| RAD23B | DEG | 1.066 | 9.218 | 44 | 82 | 9.94E+04 | 5.13E+05 | 126 |
| RAD51 | DEG | 0.679 | 0.984 | 18 | 47 | 1.07E+05 | 5.45E+05 | 65 |
| RNASEL | DEG | 0.268 | 0.692 | 6 | 4 | 1.11E+05 | 6.07E+05 | 10 |
| RPLP0 | DEG | 0.641 | 1.676 | 86 | 118 | 9.46E+04 | 4.91E+05 | 204 |
| RPS27A | DEG | 0.513 | 3.820 | 10 | 16 | 1.10E+05 | 5.78E+05 | 26 |
| RRM1 | DEG | 0.527 | 2.258 | 64 | 171 | 9.72E+04 | 4.74E+05 | 235 |
| RUVBL2 | DEG | 0.786 | 5.295 | 68 | 159 | 9.63E+04 | 4.78E+05 | 227 |
| SIRT1 | DEG | 0.137 | 0.171 | 54 | 40 | 9.83E+04 | 5.50E+05 | 94 |
| SLC2A10 | DEG | 0.176 | 0.306 | 108 | 121 | 9.25E+04 | 4.90E+05 | 229 |
| SLC2A13 | DEG | 0.574 | 2.635 | 26 | 46 | 1.04E+05 | 5.45E+05 | 72 |
| SMARCA4 | DEG | 0.290 | 3.459 | 46 | 57 | 9.94E+04 | 5.34E+05 | 103 |
| SOD1 | DEG | 0.024 | 0.087 | 66 | 51 | 9.70E+04 | 5.42E+05 | 117 |
| SOD2 | DEG | 4.002 | 5.696 | 97 | 75 | 9.34E+04 | 5.17E+05 | 172 |
| SRSF1 | DEG | 0.375 | 1.484 | 73 | 153 | 9.55E+04 | 4.79E+05 | 226 |
| SUMO1 | nonDEG | NA | NA | 10 | 17 | 1.09E+05 | 5.57E+05 | 27 |
| TP53 | nonDEG | NA | NA | 22 | 16 | 1.04E+05 | 5.63E+05 | 38 |
| TXN | DEG | 0.203 | 0.526 | 87 | 80 | 9.44E+04 | 5.14E+05 | 167 |
| UBA52 | DEG | 0.324 | 0.417 | 47 | 79 | 9.92E+04 | 5.14E+05 | 126 |
| UBB | DEG | 0.426 | 1.037 | 25 | 23 | 1.05E+05 | 5.70E+05 | 48 |
| UBE2A | DEG | 0.047 | 0.107 | 60 | 115 | 9.74E+04 | 4.92E+05 | 175 |
| UBE2I | DEG | 0.363 | 1.359 | 16 | 36 | 1.07E+05 | 5.51E+05 | 52 |
| UBE2N | DEG | 0.077 | 0.157 | 22 | 55 | 1.05E+05 | 5.37E+05 | 77 |
| UBE2S | DEG | 0.334 | 8.326 | 69 | 138 | 9.59E+04 | 4.85E+05 | 207 |
| UBQLN1 | DEG | 0.031 | 0.201 | 81 | 93 | 9.50E+04 | 5.07E+05 | 174 |
| UBXN7 | DEG | 0.418 | 1.623 | 48 | 96 | 9.90E+04 | 5.05E+05 | 144 |
| VCP | DEG | 0.131 | 0.725 | 23 | 35 | 1.05E+05 | 5.52E+05 | 58 |
| WDTC1 | nonDEG | NA | NA | 14 | 4 | 1.07E+05 | 5.98E+05 | 18 |
| XPO1 | nonDEG | NA | NA | 3 | 5 | 1.15E+05 | 5.95E+05 | 8 |
| YWHAB | DEG | 0.534 | 2.066 | 20 | 24 | 1.05E+05 | 5.70E+05 | 44 |
| YWHAQ | nonDEG | NA | NA | 24 | 15 | 1.03E+05 | 5.67E+05 | 39 |

Library of candidate genes listed by HGNC gene symbol. DEG denotes differential expression in at least one age model used in DGEA. DEGscores calculated from age groups-(ag) and decades- (d) based DGEAs were rounded to 3 decimal places. NA in either DEGscore column indicates the gene was not differentially expressed using that age model. Ranks in network-scored lists for each age model given by ag_rank and d_rank. Network scores in binary gene networks given by ag_ns and d_ns. Summing ag_rank and d_rank yields a combined rank score in Borda.

TABLE 3
Predicted age of samples in the age-reversal screen.
Predicted ages of overexpression lines from the initial perturbation screen (“Predicted Age (years)” column). The over-expressed gene (“Gene” column), the number of passages before over-expression (“Passage” column) and the chronological age of the donor (“Line Age (years)” column) is shown. Wild-type samples are denoted as “NTg” in the “Gene” column. Replicate identifiers (“Replicate” column) were randomly assigned before conducting the experiments.

| Line | Gene | Replicate | Passage | Line Age (years) | Predicted Age (years) |
|---|---|---|---|---|---|
| AG12657 | mTagBFP2 | 1 | 12 | 55 | 66.35 |
| AG12657 | mTagBFP2 | 2 | 12 | 55 | 65.56 |
| AG12657 | NTg | 1 | 9 | 55 | 48.21 |
| AG12657 | NTg | 2 | 9 | 55 | 55.65 |
| AG12657 | ACTG1 | 1 | 12 | 55 | 41.07 |
| AG12657 | ACTG1 | 2 | 12 | 55 | 60.23 |
| AG12657 | ACTL6A | 1 | 12 | 55 | 72.29 |
| AG12657 | ACTL6A | 2 | 12 | 55 | 76.65 |
| AG12657 | AKT1 | 1 | 12 | 55 | 73.91 |
| AG12657 | AKT1 | 2 | 12 | 55 | 74.24 |
| AG12657 | ATG5 | 1 | 12 | 55 | 53.55 |
| AG12657 | ATG5 | 2 | 12 | 55 | 71.83 |
| AG12657 | CCNA2 | 2 | 12 | 55 | 73.69 |
| AG12657 | CCNA2 | 1 | 12 | 55 | 72.37 |
| AG12657 | CCNB1 | 1 | 12 | 55 | 76.48 |
| AG12657 | CCNB1 | 2 | 12 | 55 | 73.07 |
| AG12657 | CDC20 | 1 | 12 | 55 | 72.94 |
| AG12657 | CDC20 | 2 | 12 | 55 | 74.38 |
| AG12657 | CDC34 | 1 | 12 | 55 | 71.39 |
| AG12657 | CDC34 | 2 | 12 | 55 | 73.12 |
| AG12657 | CDK1 | 1 | 12 | 55 | 78.95 |
| AG12657 | CDK1 | 2 | 12 | 55 | 76.12 |
| AG12657 | CDK2 | 1 | 13 | 55 | 70.47 |
| AG12657 | CDK2 | 2 | 13 | 55 | 71.29 |
| AG12657 | CDK4 | 1 | 12 | 55 | 72.02 |
| AG12657 | CDK4 | 2 | 12 | 55 | 75.76 |
| AG12657 | CDKN2A | 1 | 12 | 55 | 65.42 |
| AG12657 | CDKN2A | 2 | 12 | 55 | 71.88 |
| AG12657 | CHEK1 | 1 | 12 | 55 | 75.66 |
| AG12657 | CHEK1 | 2 | 12 | 55 | 74.8 |
| AG12657 | CHEK2 | 1 | 12 | 55 | 71.62 |
| AG12657 | CHEK2 | 2 | 12 | 55 | 66.59 |
| AG12657 | CTNNB1 | 1 | 12 | 55 | 81.29 |
| AG12657 | CTNNB1 | 2 | 12 | 55 | 77.92 |
| AG12657 | CXCR2 | 1 | 12 | 55 | 75.74 |
| AG12657 | CXCR2 | 2 | 12 | 55 | 79.24 |
| AG12657 | CYCS | 1 | 12 | 55 | 66.71 |
| AG12657 | CYCS | 2 | 12 | 55 | 70.94 |
| AG12657 | DDX3X | 1 | 12 | 55 | 77.94 |
| AG12657 | DDX3X | 2 | 12 | 55 | 76.38 |
| AG12657 | EEF2 | 1 | 12 | 55 | 77.65 |
| AG12657 | EEF2 | 2 | 12 | 55 | 82.54 |
| AG12657 | ESR1 | 1 | 12 | 55 | 78.48 |
| AG12657 | ESR1 | 2 | 12 | 55 | 80.08 |
| AG12657 | FAU | 1 | 12 | 55 | 77.16 |
| AG12657 | FAU | 2 | 12 | 55 | 77.39 |
| AG12657 | GART | 1 | 13 | 55 | 74.56 |
| AG12657 | GART | 2 | 13 | 55 | 73.66 |
| AG12657 | GSK3B | 1 | 12 | 55 | 67.29 |
| AG12657 | GSK3B | 2 | 12 | 55 | 78.88 |
| AG12657 | HDAC1 | 1 | 12 | 55 | 80.09 |
| AG12657 | HDAC1 | 2 | 12 | 55 | 72.38 |
| AG12657 | HDAC6 | 1 | 12 | 55 | 71.62 |
| AG12657 | HDAC6 | 2 | 12 | 55 | 73.1 |
| AG12657 | HES1 | 1 | 12 | 55 | 75.04 |
| AG12657 | HES1 | 2 | 12 | 55 | 70.61 |
| AG12657 | HNRNPA2B1 | 1 | 12 | 55 | 74.54 |
| AG12657 | HNRNPA2B1 | 2 | 12 | 55 | 70.55 |
| AG12657 | HSP90AB1 | 1 | 12 | 55 | 64.62 |
| AG12657 | HSP90AB1 | 2 | 12 | 55 | 71.92 |
| AG12657 | HSP90B1 | 1 | 12 | 55 | 62.84 |
| AG12657 | HSP90B1 | 2 | 12 | 55 | 72.72 |
| AG12657 | HSPA5 | 1 | 12 | 55 | 73.38 |
| AG12657 | HSPA5 | 2 | 12 | 55 | 70.64 |
| AG12657 | HSPA8 | 1 | 12 | 55 | 75.96 |
| AG12657 | HSPA8 | 2 | 12 | 55 | 72.34 |
| AG12657 | HSPD1 | 1 | 12 | 55 | 71.33 |
| AG12657 | HSPD1 | 2 | 12 | 55 | 74.7 |
| AG12657 | ISG15 | 1 | 12 | 55 | 72.27 |
| AG12657 | ISG15 | 2 | 12 | 55 | 77.97 |
| AG12657 | KAT7 | 1 | 12 | 55 | 89.85 |
| AG12657 | KAT7 | 2 | 12 | 55 | 89.01 |
| AG12657 | KDM6A | 1 | 12 | 55 | 74.05 |
| AG12657 | KDM6A | 2 | 12 | 55 | 74.63 |
| AG12657 | MAPK14 | 1 | 12 | 55 | 74.11 |
| AG12657 | MAPK14 | 2 | 12 | 55 | 70.9 |
| AG12657 | MAPK7 | 1 | 12 | 55 | 74.54 |
| AG12657 | MAPK7 | 2 | 12 | 55 | 76.4 |
| AG12657 | MCM7 | 1 | 12 | 55 | 72.2 |
| AG12657 | MCM7 | 2 | 12 | 55 | 71.26 |
| AG12657 | MDM2 | 1 | 12 | 55 | 74.67 |
| AG12657 | MDM2 | 2 | 12 | 55 | 73.64 |
| AG12657 | MTOR | 1 | 12 | 55 | 62.37 |
| AG12657 | MTOR | 2 | 12 | 55 | 68.71 |
| AG12657 | NFKB1 | 1 | 12 | 55 | 69.85 |
| AG12657 | NFKB1 | 2 | 12 | 55 | 67.94 |
| AG12657 | NOTCH1 | 1 | 12 | 55 | 75.89 |
| AG12657 | NOTCH1 | 2 | 12 | 55 | 58.58 |
| AG12657 | NPM1 | 1 | 12 | 55 | 37.38 |
| AG12657 | NPM1 | 2 | 12 | 55 | 34.27 |
| AG12657 | OGT | 1 | 13 | 55 | 73.33 |
| AG12657 | OGT | 2 | 13 | 55 | 75.75 |
| AG12657 | OSKM | 1 | 12 | 55 | 43.4 |
| AG12657 | OSKM | 2 | 12 | 55 | 71.87 |
| AG12657 | P4HB | 1 | 12 | 55 | 63.68 |
| AG12657 | P4HB | 2 | 12 | 55 | 71.8 |
| AG12657 | PARK7 | 1 | 12 | 55 | 71.89 |
| AG12657 | PARK7 | 2 | 12 | 55 | 71.76 |
| AG12657 | PIK3C2A | 1 | 12 | 55 | 82.16 |
| AG12657 | PIK3C2A | 2 | 12 | 55 | 79.18 |
| AG12657 | PIK3CA | 1 | 12 | 55 | 71.93 |
| AG12657 | PIK3CA | 2 | 12 | 55 | 72.09 |
| AG12657 | PKM | 2 | 12 | 55 | 71.01 |
| AG12657 | PKM | 1 | 12 | 55 | 67.77 |
| AG12657 | PLK1 | 1 | 12 | 55 | 74.31 |
| AG12657 | PLK1 | 2 | 12 | 55 | 73.27 |
| AG12657 | PPIA | 1 | 12 | 55 | 67.11 |
| AG12657 | PPIA | 2 | 12 | 55 | 60.18 |
| AG12657 | PPP1CA | 1 | 12 | 55 | 79.52 |
| AG12657 | PPP1CA | 2 | 12 | 55 | 74.4 |
| AG12657 | PPP1CB | 1 | 12 | 55 | 65.76 |
| AG12657 | PPP1CB | 2 | 12 | 55 | 69.76 |
| AG12657 | PPP1CC | 1 | 12 | 55 | 70.19 |
| AG12657 | PPP1CC | 2 | 12 | 55 | 68.9 |
| AG12657 | PPP2CA | 1 | 12 | 55 | 71.19 |
| AG12657 | PPP2CA | 2 | 12 | 55 | 59.7 |
| AG12657 | PPP3CA | 1 | 12 | 55 | 69.82 |
| AG12657 | PPP3CA | 2 | 12 | 55 | 74.42 |
| AG12657 | PPP4C | 1 | 12 | 55 | 73.6 |
| AG12657 | PPP4C | 2 | 12 | 55 | 70.28 |
| AG12657 | PSMC6 | 1 | 12 | 55 | 71.28 |
| AG12657 | PSMC6 | 2 | 12 | 55 | 72.35 |
| AG12657 | PSMD4 | 1 | 10 | 55 | 66.51 |
| AG12657 | PSMD4 | 2 | 10 | 55 | 55.01 |
| AG12657 | PTEN | 1 | 12 | 55 | 75.51 |
| AG12657 | PTEN | 2 | 12 | 55 | 71.83 |
| AG12657 | RAD23B | 1 | 12 | 55 | 73.58 |
| AG12657 | RAD23B | 2 | 12 | 55 | 72.15 |
| AG12657 | RAD51 | 2 | 12 | 55 | 61.62 |
| AG12657 | RAD51 | 1 | 12 | 55 | 56.71 |
| AG12657 | RNASEL | 1 | 12 | 55 | 44.44 |
| AG12657 | RNASEL | 2 | 12 | 55 | 15.8 |
| AG12657 | RPLPO | 1 | 12 | 55 | 70.5 |
| AG12657 | RPLPO | 2 | 12 | 55 | 72.99 |
| AG12657 | RPS27A | 1 | 12 | 55 | 72.4 |
| AG12657 | RPS27A | 2 | 12 | 55 | 70.34 |
| AG12657 | RRM1 | 1 | 12 | 55 | 71.43 |
| AG12657 | RRM1 | 2 | 12 | 55 | 61.96 |
| AG12657 | RUVBL2 | 1 | 12 | 55 | 70.05 |
| AG12657 | RUVBL2 | 2 | 12 | 55 | 71.77 |
| AG12657 | SIRT1 | 1 | 12 | 55 | 67.15 |
| AG12657 | SIRT1 | 2 | 12 | 55 | 73.28 |
| AG12657 | SLC2A10 | 1 | 12 | 55 | 55.88 |
| AG12657 | SLC2A10 | 2 | 12 | 55 | 57.73 |
| AG12657 | SLC2A13 | 1 | 12 | 55 | 36.84 |
| AG12657 | SLC2A13 | 2 | 12 | 55 | 47.4 |
| AG12657 | SMARCA4 | 1 | 11 | 55 | 58.69 |
| AG12657 | SMARCA4 | 2 | 11 | 55 | 66.09 |
| AG12657 | SOD1 | 1 | 12 | 55 | 77.53 |
| AG12657 | SOD1 | 2 | 12 | 55 | 70.9 |
| AG12657 | SOD2 | 1 | 12 | 55 | 69.27 |
| AG12657 | SOD2 | 2 | 12 | 55 | 71.16 |
| AG12657 | SRSF1 | 1 | 12 | 55 | 7.98 |
| AG12657 | SRSF1 | 2 | 12 | 55 | 16.31 |
| AG12657 | SUMO1 | 1 | 12 | 55 | 43.17 |
| AG12657 | SUMO1 | 2 | 12 | 55 | 49.68 |
| AG12657 | TP53 (DN) | 1 | 12 | 55 | 50.67 |
| AG12657 | TP53 (DN) | 2 | 12 | 55 | 72.61 |
| AG12657 | TXN | 1 | 12 | 55 | 71.81 |
| AG12657 | TXN | 2 | 12 | 55 | 74.33 |
| AG12657 | UBA52 | 1 | 12 | 55 | 68.35 |
| AG12657 | UBA52 | 2 | 12 | 55 | 66.23 |
| AG12657 | UBB | 1 | 12 | 55 | 75.7 |
| AG12657 | UBB | 2 | 12 | 55 | 75.49 |
| AG12657 | UBE2A | 1 | 12 | 55 | 74.66 |
| AG12657 | UBE2A | 2 | 12 | 55 | 70.57 |
| AG12657 | UBE2I | 1 | 12 | 55 | 72.95 |
| AG12657 | UBE2I | 2 | 12 | 55 | 74.84 |
| AG12657 | UBE2N | 1 | 12 | 55 | 79.9 |
| AG12657 | UBE2N | 2 | 12 | 55 | 74.61 |
| AG12657 | UBE2S | 1 | 12 | 55 | 72.16 |
| AG12657 | UBE2S | 2 | 12 | 55 | 71.68 |
| AG12657 | UBQLN1 | 1 | 12 | 55 | 38.8 |
| AG12657 | UBQLN1 | 2 | 12 | 55 | 73.69 |
| AG12657 | UBXN7 | 1 | 13 | 55 | 72 |
| AG12657 | UBXN7 | 2 | 13 | 55 | 76.34 |
| AG12657 | VCP | 1 | 13 | 55 | 76.77 |
| AG12657 | VCP | 2 | 13 | 55 | 77.09 |
| AG12657 | WDTC1 | 1 | 12 | 55 | 76.74 |
| AG12657 | WDTC1 | 2 | 12 | 55 | 72.1 |
| AG12657 | XPO1 | 1 | 12 | 55 | 62.79 |
| AG12657 | XPO1 | 2 | 12 | 55 | 66.92 |
| AG12657 | YWHAB | 1 | 12 | 55 | 67.07 |
| AG12657 | YWHAB | 2 | 12 | 55 | 74.69 |
| AG12657 | YWHAQ | 1 | 12 | 55 | 71.58 |
| AG12657 | YWHAQ | 2 | 12 | 55 | 71.78 |
| AG06294A | mTagBFP2 | 1 | 10 | 65 | 64.72 |
| AG06294A | mTagBFP2 | 2 | 10 | 65 | 64.67 |
| AG06294A | NTg | 1 | 5 | 65 | 58.61 |
| AG06294A | NTg | 2 | 5 | 65 | 65.53 |
| AG06294A | ACTL6A | 1 | 10 | 65 | 74.83 |
| AG06294A | ACTL6A | 2 | 10 | 65 | 76.28 |
| AG06294A | ATG5 | 1 | 10 | 65 | 73.44 |
| AG06294A | ATG5 | 2 | 10 | 65 | 72.63 |
| AG06294A | CCNA2 | 1 | 10 | 65 | 75.74 |
| AG06294A | CCNA2 | 2 | 10 | 65 | 76.54 |
| AG06294A | CCNB1 | 1 | 13 | 65 | 74.12 |
| AG06294A | CCNB1 | 2 | 13 | 65 | 65.73 |
| AG06294A | CDC20 | 2 | 10 | 65 | 74.84 |
| AG06294A | CDC20 | 1 | 10 | 65 | 74.42 |
| AG06294A | CDK1 | 1 | 10 | 65 | 72.3 |
| AG06294A | CDK1 | 2 | 10 | 65 | 70.97 |
| AG06294A | CDKN2A | 1 | 10 | 65 | 78.64 |
| AG06294A | CDKN2A | 2 | 10 | 65 | 75.35 |
| AG06294A | CHEK2 | 1 | 10 | 65 | 73.65 |
| AG06294A | CHEK2 | 2 | 10 | 65 | 61.23 |
| AG06294A | CXCR2 | 1 | 10 | 65 | 78.92 |
| AG06294A | CXCR2 | 2 | 10 | 65 | 75.83 |
| AG06294A | EEF2 | 1 | 10 | 65 | 70.75 |
| AG06294A | EEF2 | 2 | 10 | 65 | 80.24 |
| AG06294A | ESR1 | 1 | 10 | 65 | 77.45 |
| AG06294A | ESR1 | 2 | 10 | 65 | 83.83 |
| AG06294A | GSK3B | 1 | 12 | 65 | 74.02 |
| AG06294A | GSK3B | 2 | 12 | 65 | 72.37 |
| AG06294A | HES1 | 1 | 10 | 65 | 73.06 |
| AG06294A | HES1 | 2 | 10 | 65 | 74.4 |
| AG06294A | ISG15 | 1 | 10 | 65 | 77.47 |
| AG06294A | ISG15 | 2 | 10 | 65 | 74.08 |
| AG06294A | KAT7 | 1 | 10 | 65 | 91.9 |
| AG06294A | KAT7 | 2 | 10 | 65 | 89.45 |
| AG06294A | LRRK2 | 1 | 9 | 65 | 74.85 |
| AG06294A | LRRK2 | 2 | 9 | 65 | 71.18 |
| AG06294A | MAPK7 | 1 | 9 | 65 | 77.95 |
| AG06294A | MAPK7 | 2 | 9 | 65 | 79.14 |
| AG06294A | MDM2 | 1 | 10 | 65 | 75.79 |
| AG06294A | MDM2 | 2 | 10 | 65 | 76.42 |
| AG06294A | NFKB1 | 1 | 9 | 65 | 66.02 |
| AG06294A | NFKB1 | 2 | 9 | 65 | 76.28 |
| AG06294A | OGT | 1 | 10 | 65 | 72.87 |
| AG06294A | OGT | 2 | 10 | 65 | 61.04 |
| AG06294A | OSKM | 1 | 9 | 65 | 52.08 |
| AG06294A | OSKM | 2 | 9 | 65 | 56.33 |
| AG06294A | P4HB | 1 | 10 | 65 | 62.65 |
| AG06294A | P4HB | 2 | 10 | 65 | 74.74 |
| AG06294A | PARK7 | 1 | 10 | 65 | 73.19 |
| AG06294A | PARK7 | 2 | 10 | 65 | 78.56 |
| AG06294A | PIK3C2A | 1 | 9 | 65 | 73.96 |
| AG06294A | PIK3C2A | 2 | 9 | 65 | 77.87 |
| AG06294A | PIK3C3 | 1 | 9 | 65 | 69.67 |
| AG06294A | PIK3C3 | 2 | 9 | 65 | 75.43 |
| AG06294A | PIK3CA | 1 | 9 | 65 | 64.56 |
| AG06294A | PIK3CA | 2 | 9 | 65 | 73.61 |
| AG06294A | PKM | 1 | 10 | 65 | 70.47 |
| AG06294A | PKM | 2 | 10 | 65 | 78.47 |
| AG06294A | PPIA | 1 | 10 | 65 | 71.86 |
| AG06294A | PPIA | 2 | 10 | 65 | 79.41 |
| AG06294A | PPP1CA | 1 | 10 | 65 | 78.36 |
| AG06294A | PPP1CA | 2 | 10 | 65 | 77.31 |
| AG06294A | PPP1CB | 1 | 10 | 65 | 78.51 |
| AG06294A | PPP1CB | 2 | 10 | 65 | 74.23 |
| AG06294A | PPP1CC | 1 | 10 | 65 | 74.1 |
| AG06294A | PPP1CC | 2 | 10 | 65 | 78.25 |
| AG06294A | PPP3CA | 1 | 9 | 65 | 72.45 |
| AG06294A | PPP3CA | 2 | 9 | 65 | 71.34 |
| AG06294A | PPP4C | 1 | 10 | 65 | 75.45 |
| AG06294A | PPP4C | 2 | 10 | 65 | 74.73 |
| AG06294A | PSMC6 | 1 | 10 | 65 | 76 |
| AG06294A | PSMC6 | 2 | 10 | 65 | 81.77 |
| AG06294A | PTEN | 1 | 10 | 65 | 78.3 |
| AG06294A | PTEN | 2 | 10 | 65 | 75.76 |
| AG06294A | RAD23B | 1 | 10 | 65 | 72.42 |
| AG06294A | RAD23B | 2 | 10 | 65 | 70.43 |
| AG06294A | RPLPO | 1 | 10 | 65 | 70.61 |
| AG06294A | RPLPO | 2 | 10 | 65 | 68.27 |
| AG06294A | RPS27A | 1 | 10 | 65 | 72.4 |
| AG06294A | RPS27A | 2 | 10 | 65 | 70.6 |
| AG06294A | SIRT1 | 1 | 9 | 65 | 52.62 |
| AG06294A | SIRT1 | 2 | 9 | 65 | 61.63 |
| AG06294A | SLC2A10 | 1 | 10 | 65 | 75.26 |
| AG06294A | SLC2A10 | 2 | 10 | 65 | 70.02 |
| AG06294A | SLC2A13 | 1 | 10 | 65 | 63.84 |
| AG06294A | SLC2A13 | 2 | 10 | 65 | 70.05 |
| AG06294A | SOD1 | 1 | 10 | 65 | 65.53 |
| AG06294A | SOD1 | 2 | 10 | 65 | 76.49 |
| AG06294A | SOD2 | 1 | 10 | 65 | 76.12 |
| AG06294A | SOD2 | 2 | 10 | 65 | 80.12 |
| AG06294A | SRSF1 | 1 | 10 | 65 | 24.41 |
| AG06294A | SRSF1 | 2 | 10 | 65 | 14.32 |
| AG06294A | TP53 (DN) | 1 | 10 | 65 | 72.22 |
| AG06294A | TP53 (DN) | 2 | 10 | 65 | 70.88 |
| AG06294A | TXN | 1 | 10 | 65 | 75.92 |
| AG06294A | TXN | 2 | 10 | 65 | 73.03 |
| AG06294A | UBA52 | 1 | 10 | 65 | 73.89 |
| AG06294A | UBA52 | 2 | 10 | 65 | 75.13 |
| AG06294A | UBB | 1 | 10 | 65 | 73 |
| AG06294A | UBB | 2 | 10 | 65 | 64.67 |
| AG06294A | UBE2S | 1 | 10 | 65 | 78.94 |
| AG06294A | UBE2S | 2 | 10 | 65 | 79.64 |
| AG06294A | UBQLN1 | 2 | 10 | 65 | 71.22 |
| AG06294A | UBQLN1 | 1 | 10 | 65 | 78.88 |
| AG06294A | WDTC1 | 1 | 9 | 65 | 74.15 |
| AG06294A | WDTC1 | 2 | 9 | 65 | 76.55 |
| AG06294A | YWHAB | 1 | 10 | 65 | 75.55 |
| AG06294A | YWHAB | 2 | 10 | 65 | 72.2 |
| AG06294A | YWHAQ | 1 | 10 | 65 | 72.6 |
| AG06294A | YWHAQ | 2 | 10 | 65 | 75.06 |
| AG16359 | mTagBFP2 | 1 | 13 | 79 | 78.78 |
| AG16359 | mTagBFP2 | 2 | 13 | 79 | 72.05 |
| AG16359 | NTg | 1 | 10 | 79 | 82.83 |
| AG16359 | NTg | 2 | 10 | 79 | 75.12 |
| AG16359 | ACTG1 | 1 | 13 | 79 | 77.26 |
| AG16359 | ACTG1 | 2 | 13 | 79 | 75.2 |
| AG16359 | ACTL6A | 1 | 13 | 79 | 69.35 |
| AG16359 | ACTL6A | 2 | 13 | 79 | 70.19 |
| AG16359 | AKT1 | 1 | 13 | 79 | 81.72 |
| AG16359 | AKT1 | 2 | 13 | 79 | 82.24 |
| AG16359 | ATG5 | 1 | 13 | 79 | 72.4 |
| AG16359 | ATG5 | 2 | 13 | 79 | 74.04 |
| AG16359 | CCNA2 | 2 | 13 | 79 | 74.27 |
| AG16359 | CCNA2 | 1 | 13 | 79 | 76.33 |
| AG16359 | CCNB1 | 1 | 13 | 79 | 73.65 |
| AG16359 | CCNB1 | 2 | 13 | 79 | 77.41 |
| AG16359 | CDC20 | 1 | 13 | 79 | 74.37 |
| AG16359 | CDC20 | 2 | 13 | 79 | 74.46 |
| AG16359 | CDC34 | 1 | 13 | 79 | 74.7 |
| AG16359 | CDC34 | 2 | 13 | 79 | 71.33 |
| AG16359 | CDK1 | 1 | 13 | 79 | 76.23 |
| AG16359 | CDK1 | 2 | 13 | 79 | 77.82 |
| AG16359 | CDK2 | 1 | 13 | 79 | 76.28 |
| AG16359 | CDK2 | 2 | 13 | 79 | 78.73 |
| AG16359 | CDK4 | 1 | 13 | 79 | 73.28 |
| AG16359 | CDK4 | 2 | 13 | 79 | 70.68 |
| AG16359 | CDKN2A | 1 | 13 | 79 | 78.45 |
| AG16359 | CDKN2A | 2 | 13 | 79 | 76.17 |
| AG16359 | CHEK1 | 1 | 13 | 79 | 77.32 |
| AG16359 | CHEK1 | 2 | 13 | 79 | 80.61 |
| AG16359 | CHEK2 | 1 | 13 | 79 | 73.01 |
| AG16359 | CHEK2 | 2 | 13 | 79 | 73.23 |
| AG16359 | CTNNB1 | 1 | 13 | 79 | 79.58 |
| AG16359 | CTNNB1 | 2 | 13 | 79 | 75.8 |
| AG16359 | CXCR2 | 1 | 13 | 79 | 62.12 |
| AG16359 | CXCR2 | 2 | 13 | 79 | 79.64 |
| AG16359 | CYCS | 1 | 13 | 79 | 73.73 |
| AG16359 | CYCS | 2 | 13 | 79 | 74.81 |
| AG16359 | DDX3X | 1 | 13 | 79 | 72.69 |
| AG16359 | DDX3X | 2 | 13 | 79 | 78.34 |
| AG16359 | DICER1 | 1 | 13 | 79 | 80.16 |
| AG16359 | DICER1 | 2 | 13 | 79 | 80.31 |
| AG16359 | EEF2 | 1 | 13 | 79 | 82.91 |
| AG16359 | EEF2 | 2 | 13 | 79 | 83.95 |
| AG16359 | ESR1 | 1 | 13 | 79 | 88.87 |
| AG16359 | ESR1 | 2 | 13 | 79 | 85.62 |
| AG16359 | FAU | 1 | 13 | 79 | 70.02 |
| AG16359 | FAU | 2 | 13 | 79 | 73.05 |
| AG16359 | GART | 1 | 13 | 79 | 75.79 |
| AG16359 | GART | 2 | 13 | 79 | 58.86 |
| AG16359 | GSK3B | 1 | 13 | 79 | 74.42 |
| AG16359 | GSK3B | 2 | 13 | 79 | 80.31 |
| AG16359 | HDAC1 | 1 | 13 | 79 | 71 |
| AG16359 | HDAC1 | 2 | 13 | 79 | 76.55 |
| AG16359 | HDAC6 | 1 | 13 | 79 | 71.33 |
| AG16359 | HDAC6 | 2 | 13 | 79 | 70.99 |
| AG16359 | HES1 | 1 | 13 | 79 | 71.35 |
| AG16359 | HES1 | 2 | 13 | 79 | 79.19 |
| AG16359 | HNRNPA2B1 | 1 | 13 | 79 | 74.68 |
| AG16359 | HNRNPA2B1 | 2 | 13 | 79 | 74.62 |
| AG16359 | HSP90AB1 | 1 | 13 | 79 | 78.67 |
| AG16359 | HSP90AB1 | 2 | 13 | 79 | 73.23 |
| AG16359 | HSP90B1 | 1 | 13 | 79 | 73.42 |
| AG16359 | HSP90B1 | 2 | 13 | 79 | 73.43 |
| AG16359 | HSPA5 | 1 | 13 | 79 | 83.18 |
| AG16359 | HSPA5 | 2 | 13 | 79 | 85.55 |
| AG16359 | HSPA8 | 1 | 13 | 79 | 77.09 |
| AG16359 | HSPA8 | 2 | 13 | 79 | 77.59 |
| AG16359 | HSPD1 | 1 | 13 | 79 | 79.53 |
| AG16359 | HSPD1 | 2 | 13 | 79 | 75.55 |
| AG16359 | ISG15 | 1 | 13 | 79 | 76.13 |
| AG16359 | ISG15 | 2 | 13 | 79 | 78.42 |
| AG16359 | KAT7 | 1 | 13 | 79 | 92.21 |
| AG16359 | KAT7 | 2 | 13 | 79 | 97.86 |
| AG16359 | KDM6A | 1 | 13 | 79 | 82.23 |
| AG16359 | KDM6A | 2 | 13 | 79 | 82.68 |
| AG16359 | LRRK2 | 1 | 13 | 79 | 73.83 |
| AG16359 | LRRK2 | 2 | 13 | 79 | 66.91 |
| AG16359 | MAPK14 | 1 | 13 | 79 | 78.32 |
| AG16359 | MAPK14 | 2 | 13 | 79 | 76.91 |
| AG16359 | MAPK7 | 1 | 13 | 79 | 83.06 |
| AG16359 | MAPK7 | 2 | 13 | 79 | 81.97 |
| AG16359 | MCM7 | 1 | 13 | 79 | 74.19 |
| AG16359 | MCM7 | 2 | 13 | 79 | 76.95 |
| AG16359 | MDM2 | 1 | 13 | 79 | 65.69 |
| AG16359 | MDM2 | 2 | 13 | 79 | 75.71 |
| AG16359 | NFKB1 | 1 | 13 | 79 | 67.34 |
| AG16359 | NFKB1 | 2 | 13 | 79 | 77.6 |
| AG16359 | NOTCH1 | 1 | 13 | 79 | 81.92 |
| AG16359 | NOTCH1 | 2 | 13 | 79 | 76.95 |
| AG16359 | NPM1 | 1 | 13 | 79 | 76.98 |
| AG16359 | NPM1 | 2 | 13 | 79 | 84.5 |
| AG16359 | OGT | 1 | 13 | 79 | 76.36 |
| AG16359 | OGT | 2 | 13 | 79 | 70.21 |
| AG16359 | OSKM | 1 | 13 | 79 | 70.74 |
| AG16359 | OSKM | 2 | 13 | 79 | 62.85 |
| AG16359 | P4HB | 1 | 13 | 79 | 66.13 |
| AG16359 | P4HB | 2 | 13 | 79 | 60.8 |
| AG16359 | PARK7 | 1 | 13 | 79 | 72.95 |
| AG16359 | PARK7 | 2 | 13 | 79 | 78.13 |
| AG16359 | PIK3C2A | 1 | 13 | 79 | 78.2 |
| AG16359 | PIK3C2A | 2 | 13 | 79 | 74.94 |
| AG16359 | PIK3CA | 2 | 13 | 79 | 54.84 |
| AG16359 | PIK3CA | 1 | 13 | 79 | 75.58 |
| AG16359 | PKM | 1 | 13 | 79 | 79.55 |
| AG16359 | PKM | 2 | 13 | 79 | 74.18 |
| AG16359 | PLK1 | 1 | 13 | 79 | 79.96 |
| AG16359 | PLK1 | 2 | 13 | 79 | 72.99 |
| AG16359 | PPIA | 1 | 13 | 79 | 80.56 |
| AG16359 | PPIA | 2 | 13 | 79 | 75.32 |
| AG16359 | PPP1CA | 1 | 13 | 79 | 72.07 |
| AG16359 | PPP1CA | 2 | 13 | 79 | 77.87 |
| AG16359 | PPP1CB | 1 | 13 | 79 | 74.53 |
| AG16359 | PPP1CB | 2 | 13 | 79 | 67.21 |
| AG16359 | PPP1CC | 1 | 13 | 79 | 74.71 |
| AG16359 | PPP1CC | 2 | 13 | 79 | 76.6 |
| AG16359 | PPP2CA | 1 | 13 | 79 | 81.91 |
| AG16359 | PPP2CA | 2 | 13 | 79 | 71.45 |
| AG16359 | PPP3CA | 1 | 13 | 79 | 77.71 |
| AG16359 | PPP3CA | 2 | 13 | 79 | 72.9 |
| AG16359 | PPP4C | 1 | 13 | 79 | 74.91 |
| AG16359 | PPP4C | 2 | 13 | 79 | 79.22 |
| AG16359 | PSMC6 | 1 | 13 | 79 | 77.26 |
| AG16359 | PSMC6 | 2 | 13 | 79 | 72.26 |
| AG16359 | PSMD4 | 1 | 13 | 79 | 81.51 |
| AG16359 | PSMD4 | 2 | 13 | 79 | 84.09 |
| AG16359 | PTEN | 1 | 13 | 79 | 78.22 |
| AG16359 | PTEN | 2 | 13 | 79 | 76.25 |
| AG16359 | RAD23B | 1 | 13 | 79 | 69.73 |
| AG16359 | RAD23B | 2 | 13 | 79 | 72.11 |
| AG16359 | RAD51 | 1 | 13 | 79 | 76.79 |
| AG16359 | RAD51 | 2 | 13 | 79 | 74.77 |
| AG16359 | RNASEL | 1 | 13 | 79 | 77.37 |
| AG16359 | RNASEL | 2 | 13 | 79 | 77.84 |
| AG16359 | RPLPO | 1 | 13 | 79 | 72.7 |
| AG16359 | RPLPO | 2 | 13 | 79 | 81.43 |
| AG16359 | RPS27A | 1 | 13 | 79 | 77.6 |
| AG16359 | RPS27A | 2 | 13 | 79 | 78.6 |
| AG16359 | RRM1 | 1 | 13 | 79 | 79.62 |
| AG16359 | RRM1 | 2 | 13 | 79 | 75.56 |
| AG16359 | RUVBL2 | 1 | 13 | 79 | 74.87 |
| AG16359 | RUVBL2 | 2 | 13 | 79 | 76.36 |
| AG16359 | SIRT1 | 1 | 13 | 79 | 71.7 |
| AG16359 | SIRT1 | 2 | 13 | 79 | 70.96 |
| AG16359 | SLC2A10 | 1 | 13 | 79 | 72.49 |
| AG16359 | SLC2A10 | 2 | 13 | 79 | 82.28 |
| AG16359 | SLC2A13 | 1 | 13 | 79 | 75.18 |
| AG16359 | SLC2A13 | 2 | 13 | 79 | 80.31 |
| AG16359 | SOD1 | 1 | 13 | 79 | 81.12 |
| AG16359 | SOD1 | 2 | 13 | 79 | 78.21 |
| AG16359 | SOD2 | 1 | 13 | 79 | 76.56 |
| AG16359 | SOD2 | 2 | 13 | 79 | 74.34 |
| AG16359 | SRSF1 | 1 | 13 | 79 | 51 |
| AG16359 | SRSF1 | 2 | 13 | 79 | 51.06 |
| AG16359 | SUMO1 | 1 | 13 | 79 | 81.35 |
| AG16359 | SUMO1 | 2 | 13 | 79 | 76.68 |
| AG16359 | TP53 (DN) | 1 | 13 | 79 | 79.57 |
| AG16359 | TP53 (DN) | 2 | 13 | 79 | 79.43 |
| AG16359 | TXN | 1 | 13 | 79 | 82.81 |
| AG16359 | TXN | 2 | 13 | 79 | 84.44 |
| AG16359 | UBA52 | 1 | 13 | 79 | 80.68 |
| AG16359 | UBA52 | 2 | 13 | 79 | 72.71 |
| AG16359 | UBB | 1 | 13 | 79 | 70.71 |
| AG16359 | UBB | 2 | 13 | 79 | 72 |
| AG16359 | UBE2A | 1 | 13 | 79 | 76.5 |
| AG16359 | UBE2A | 2 | 13 | 79 | 69.85 |
| AG16359 | UBE2I | 1 | 13 | 79 | 76.65 |
| AG16359 | UBE2I | 2 | 13 | 79 | 82.14 |
| AG16359 | UBE2N | 1 | 13 | 79 | 78.13 |
| AG16359 | UBE2N | 2 | 13 | 79 | 83.47 |
| AG16359 | UBE2S | 1 | 13 | 79 | 81.37 |
| AG16359 | UBE2S | 2 | 13 | 79 | 78.38 |
| AG16359 | UBQLN1 | 1 | 13 | 79 | 73.62 |
| AG16359 | UBQLN1 | 2 | 13 | 79 | 78.77 |
| AG16359 | VCP | 1 | 13 | 79 | 78.45 |
| AG16359 | VCP | 2 | 13 | 79 | 79.41 |
| AG16359 | WDTC1 | 1 | 13 | 79 | 1.78 |
| AG16359 | WDTC1 | 2 | 13 | 79 | 75.8 |
| AG16359 | XPO1 | 1 | 13 | 79 | 78.6 |
| AG16359 | XPO1 | 2 | 13 | 79 | 80.13 |
| AG16359 | YWHAB | 1 | 13 | 79 | 74.55 |
| AG16359 | YWHAB | 2 | 13 | 79 | 80.58 |
| AG16359 | YWHAQ | 1 | 13 | 79 | 74.95 |
| AG16359 | YWHAQ | 2 | 13 | 79 | 79.1 |

### Example 2

### Srsf1 Induces Robust Cellular Rejuvenation

To validate the initial findings and further study the effect of the hits on the aging phenotype, the experiment was repeated by generating new overexpression lines in NHDFs from 6 different donors (M55, M65, M67, M68, M69, M79). In agreement with the initial results, most of the genes were observed to have cell-line specific effects, but some of them lost the effect in the transcriptomic assay (FIG. 4A, Table 3). However, SRSF1 displayed robust transcriptomic age reversal in all 6 lines, with an average rejuvenation effect of approximately 26 years, which was expected considering its strong effect on the clock genes (FIG. 3B). The functional assays showed that SRSF1 induction reduced cellular senescence by more than 50% across all 6 lines, suggesting functional rejuvenation of these cells (FIG. 4B). However, no significant effect on the mitochondrial potential was observed, in agreement with the initial screening data. Proteasome activity was reduced in the SRSF1 samples, matching the observed OSKM induction phenotype and opposite to the pro-aging effect of KAT7 and CDKN2A (FIG. 2D). To further assess rejuvenation upon SRSF1 induction, wound healing efficacy, a key function of dermal fibroblasts, was tested using an in vitro scratch assay. The data showed that SRSF1-expressing cells reached the midpoint of wound closure 50% faster than the BFP control, while OSKM seemed to slightly delay wound closure (FIG. 4C), in agreement with previous mouse wound healing results.

The effect of SRSF1 overexpression on the individual processes of the TIME predictor was then studied, and a coordinated rejuvenation was observed across all cell lines of the gene expression associated with histone methylation and translation initiation (FIG. 4C). Since histone methylation has a central role in regulating gene expression, it is expected that a reversal of the aging associated transcriptional changes of this process would have a large and robust beneficial effect on cells. Conversely, overexpression of KDM6A was observed to have a similar coordinated but detrimental effect on the histone methylation and WNT signaling processes (data not shown). Considering the strong effect of both OSKM and SRSF1 on the transcriptome, and the known dedifferentiation potential of the Yamanaka factors, whether SRSF1 also induces a loss of cell identity was also considered. By performing an enrichment analysis for fibroblast, myofibroblast, and mesenchymal stem cell (MSC) identity genes, both SRSF1 and OSKM were discovered to lead to a reduction of fibroblast cell identity when compared to the WT and BFP controls (data not shown).

Since SRSF1 is a known splicing factor, a differential splicing analysis was conducted in the overexpression lines and great agreement was observed in the distribution of splicing events between the 6 lines, with an alternative first exon accounting for more than 50% of all differential isoforms (data not shown). Interestingly, when the parent processes for the alternatively spliced genes were observed, it was discovered that histone methylation and large ribosomal subunit organization were the only processes common for all 6 lines (data not shown). Moreover, the alternatively spliced genes have been previously confirmed to directly interact with SRSF1, providing further evidence for the link between SRSF1 and gene expression changes in histone methylation and translation initiation. Therefore, a mechanism by which SRSF1 overexpression leads to differential splicing of important regulators of histone methylation and protein translation that induce youthful gene expression profiles in these processes is proposed (data not shown).

### Example 3

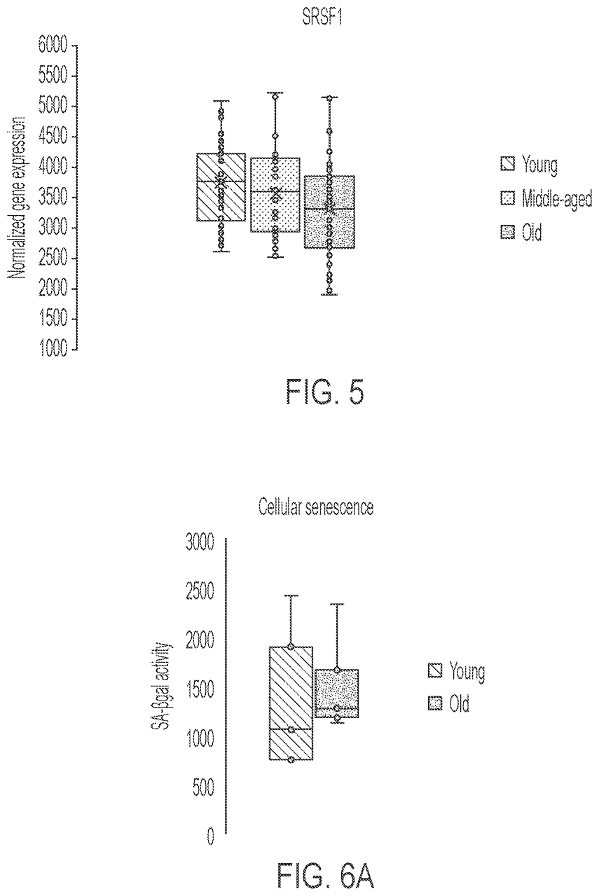

Serine and arginine rich splicing factor 1 (SRSF1) is an essential sequence specific splicing factor, whose expression decreases with aging in primary normal human dermal fibroblasts (NHDF) (FIG. 5). SRSF1 was predicted to be an age modulating gene by the target prediction algorithm, and the data above showed that its overexpression reverses transcriptomic age-related changes, as well as senescence and proteasome dysfunction in multiple aged NHDFs.

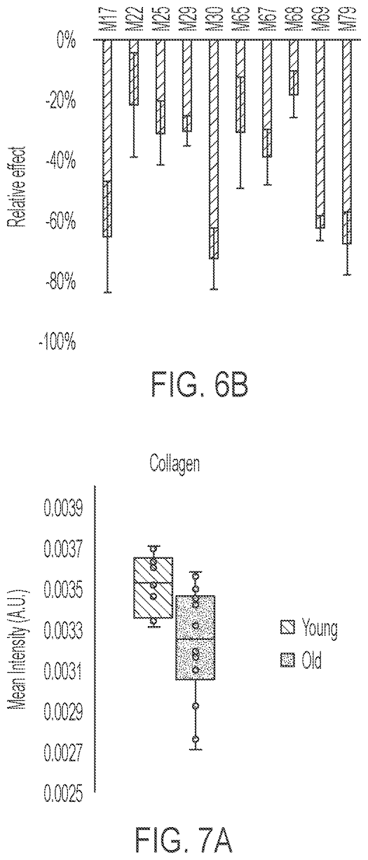

Cellular senescence is a known age-related cellular dysfunction that can be measured by the established senescence-associated β-galactosidase (SA-βgal) assay (FIG. 6A). It was further confirmed that SRSF1 activation through mRNA transfection has a senescence reducing effect in both young and old NHDFs (FIG. 6B), achieving an age reversal effect in the treated cells.

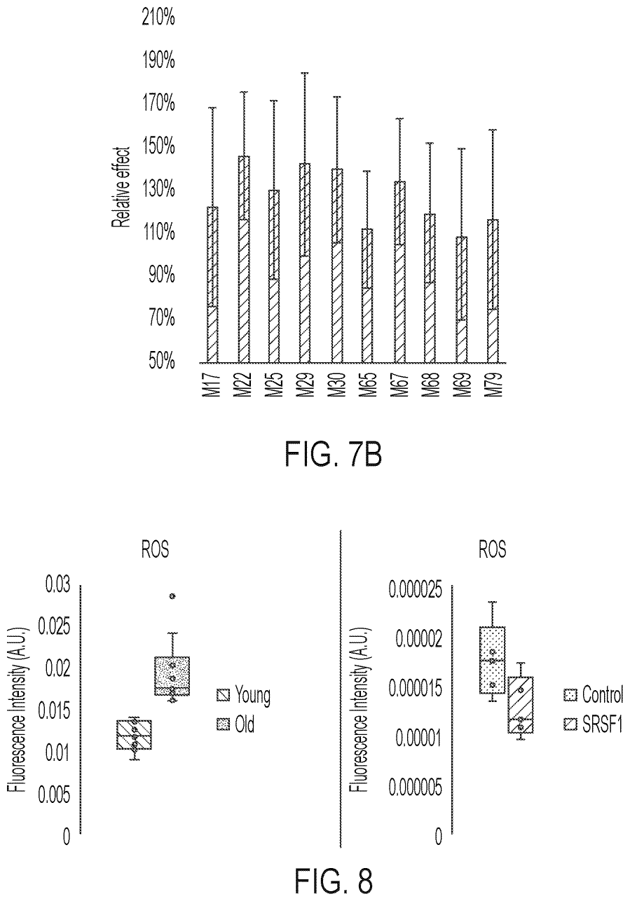

Type I collagen is one of the most abundant proteins of the extracellular matrix, serving as an important structural component of multiple tissues such as bone, skin, and heart. With aging, human fibroblasts produce less collagen (FIG. 7A), leading to cellular and tissue dysfunction. Using SRSF1 mRNA transfection, the collagen production was increased in both young and old NHDFs by 10-45% (FIG. 7B), thereby reversing this aging phenotype.

A lack of resiliency to oxidative stress is another age-related cellular dysfunction, which can be assayed by measuring reactive oxygen species (ROS) levels in response to H₂O₂ treatment. Here, the data show that older cells are less capable than the younger ones to clear ROS upon oxidative stress (FIG. 8—Left), and that SRSF1 mRNA can rescue this phenotype in old NHDFs (FIG. 8—Right).

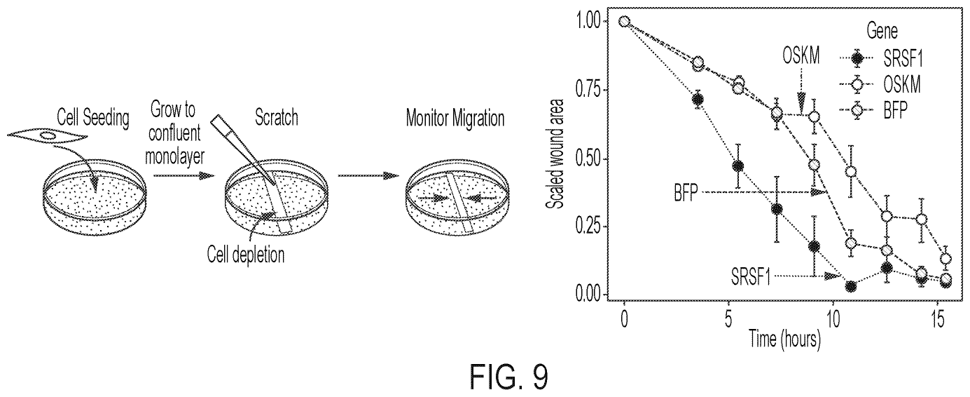

NHDF play a pivotal role in wound healing, by migrating and closing the open tissue area. This function becomes dysregulated in old cells, and it can be measured in vitro using the established scratch assay. The data showed that SRSF1-expressing cells reached the midpoint of wound closure 50% faster than the BFP control, while the Yamanaka factors (OSKM) overexpressed seemed to slightly delay wound closure, in agreement with previous mouse wound healing studies (FIG. 9 and data not shown (i.e., bright-field images of SRSF1-, OSKM-, or BFP-induced cells at 0 and 6.5 h after scratch)). Given these results, it was determined that activation of SRSF1 accelerates wound closure in vitro.

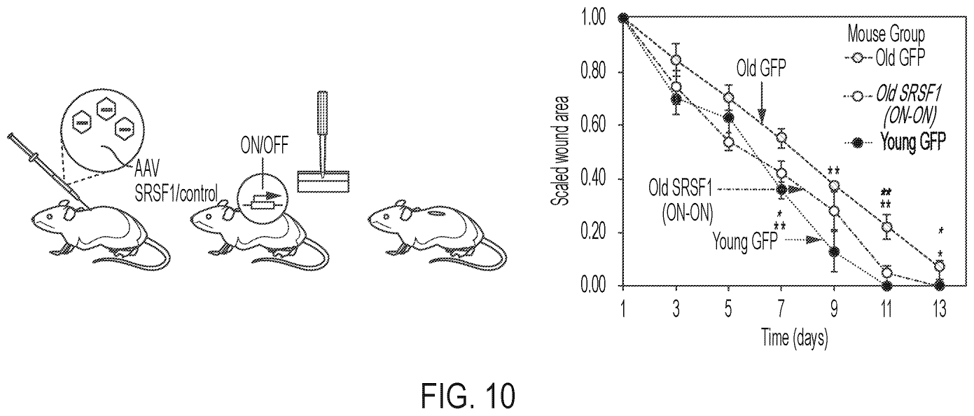

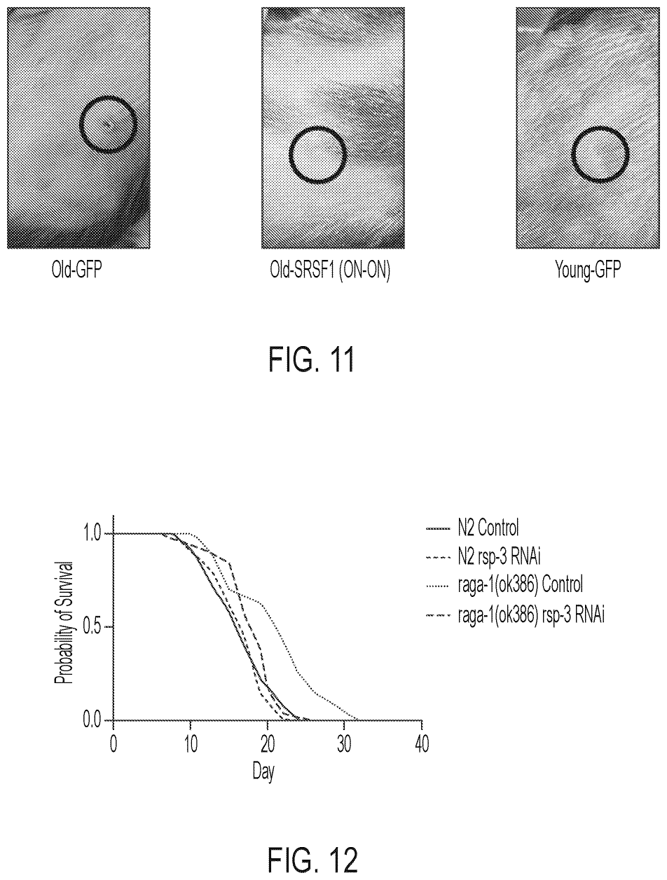

To further validate SRSF1 activation as an effective cellular rejuvenation intervention, its effect on wound healing were tested in vivo. Transgene delivery was achieved by intradermal injections of adeno-associated virus (AAV) containing SRSF1 or GFP under the control of a tetracycline-controlled (Tet-On) inducible promoter. Mice received excisional wounds via a punch biopsy and wound area was measured every 2 days. In old mice, SRSF1 overexpression during wound healing increases wound closure rate relative to GFP overexpression. Furthermore, old mice overexpressing SRSF1 reached full wound closure by the same time point as young control mice. See FIGS. 10-11. These results indicate that induction of SRSF1 accelerates wound closure in old mice, highlighting SRSF1 as a potential therapeutic target for dermal wound healing.

To evaluate the effect of SRSF1 on longevity, lifespan assays were performed in wild-type and long-lived (raga-1) worm strains, with and without knockdown of rsp-3, the worm homolog of SRSF1. The data showed that rsp-3 knockdown has no effect on WT worms, but it suppresses lifespan extension in the raga-1 strain, thereby linking rsp-3 to worm longevity (FIG. 12).

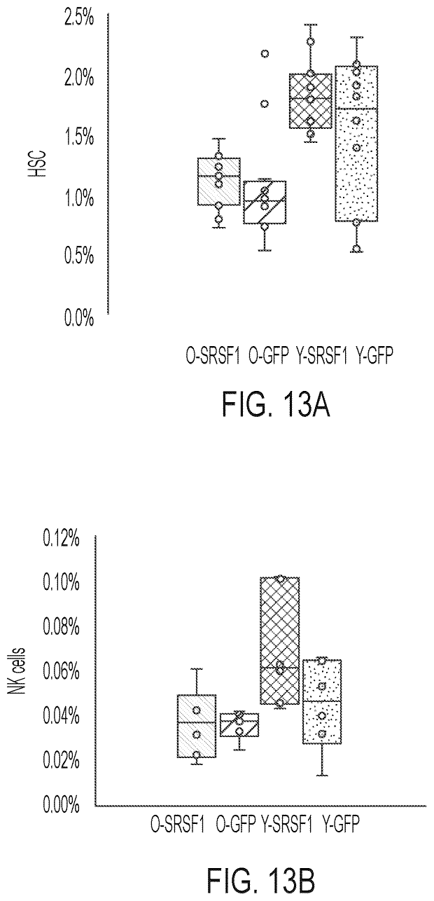

With aging, hematopoietic stem cells (HSC) lose their self-renewal capacity as well as their lymphoid output. These cellular dysfunctions lead to several age-related hematological malignancies. Data showed that mRNA electroporation of SRSF1 increases the HSC population in vitro and their ability to differentiate towards the lymphoid lineage (FIGS. 13A-13B).

### Methods

### Rna-Seq Data Processing and Analysis

Raw RNA-Seq reads were aligned to the GRCh38 human genome using STAR v2.5.2b and checked for quality control using FastQC v0.11.5. The alignment files were then indexed using SAMtools v1.3.1 and mapped reads were counted using featureCounts from the Subread v2.0.1 package. To reduce the variation between the samples caused by disproportionate sequencing depth, the fastq files were downsampled to 20M reads using Seqtk-1.3. For the overexpression analysis, to address the high number of discarded multi-mapped reads due to the sequence similarity between the endogenous genes and the barcoded genes, reads were aligned to a genome index that contains the transgene sequences (including their barcodes) using kallisto v.0.46.2, and the tximport package was used to generate TPM from estimated counts. Barcode search was performed on the downsampled fastq files by using the agrep tool for approximate string matching with 3 mismatches. Subsequent differential expression analysis was performed using DESeq2 v1.32.0 unless otherwise noted.

### Differential Gene Expression Analysis and Identification of Cell Line Identity Genes

Differential gene expression analysis was performed using the DESeq2 R package v1.30.1. In particular, DESeq data objects were constructed from raw, untransformed read counts and a design formula reflecting the attempted comparisons. In particular, to detect differentially expressed genes in response to perturbation effects within the screening data, samples of each line were separated and individually processed. Differentially expressed genes were obtained by first running the “DESeq” function with standard parameters followed by the “contrast” function to obtain differentially expressed genes between overexpressed genes and wildtype or blue fluorescent protein (BFP)-transduced controls. For each gene, computed p-values were corrected by applying Bonferroni correction and significance was determined at the 1% level. Cell line identity genes within the large-scale screening assay in three cell lines as well as the validation assay composed of six lines were identified by the following steps. First, all pairwise differentially expressed genes between wild type lines are obtained, as described before. Second, a matrix composed of log 2 fold changes across cell lines and all differentially expressed genes was constructed. Next, for each gene in all cell lines, the average log 2 fold change between the cell line under consideration and all other lines was computed. Finally, identity genes of a cell line are defined as having the lowest or highest average log 2 fold change compared to the other lines.

### Differential Splicing Analysis

Differential splicing analysis was performed using SUPPA2. In particular, the ‘generateEvents’ subcommand of SUPPA on the GENCODE V29 annotation and parameter setting ‘-e SE SS MX RI FL-f ioe’ was invoked to generate all potential splicing events. Next, the proportion spliced-in (PSI) for all potential events was generated by invoking the ‘psiPerEvent’ subcommand on the transcript-aligned, large-scale screening assay (including 6 NHDF lines with SRSF1 over-expression) RNA-seq data in TPM format. Finally, the ‘diffSplice’ command was invoked with an empirical model (‘-m empirical’) to compare changes between wildtype and SRSF1 over-expressing lines. Automatic gene correction of p-values is applied by supplying the ‘-gc’ parameter.

### Cell Type Specific Marker Enrichment

RNA-seq samples are deconvoluted into individual cell types using CibersortX. First, droplet-based single-cell reference datasets of stromal cells have been collected from Tabula Sapiens. Subsequently, for each population, 100 cells were randomly sampled and combined into a single matrix. A signature matrix containing 300 to 500 genes per cell type was computed using CibersortX. RNA-seq counts of the initial perturbation screen served as an input for deconvolution. CibersortX was run in “absolute mode” to allow for comparison between samples.

Identification of Cellular Processes that are Predictive of Age

The identification of processes that are predictive of chronological age is based on the Molecular Biology of the Cell (MBotC) Ontology, a previously curated resource relating genes to cellular processes. The MBotC Ontology is organized in four different layers where broader categories are iteratively split into more and more specialized functions.

Therefore, the third layer was empirically selected and a set of processes that are potentially informative about aging were created. In order to quantify the predictability of chronological age from these processes, “weak” age predictors for each process were built as follows: (1) Subset the gene expression matrix to genes in the process under consideration; (2) Perform principal component analysis (PCA) on the subset matrix and replace it with a matrix containing all principal components. PCA is performed using the implementation in the h2o v3.36.0.2 R package and imbalanced contribution of individual genes is alleviated by standardizing the data through the ‘transform’ parameter; (3) Train a generalized linear model (GLM) with 5-fold cross-validation using the ‘h2o.glm’ function in the h2o R package. In particular, the model is based on Gaussian distributions with an identity link function, which standardizes the input before training and implements automatic lambda search with ridge regression; (4) Train a “strong” age predictor based on the in-bag predictions of all weak predictors. Like weak predictors, the strong age predictor is a GLM with 10-fold cross-validation that is based on Gaussian distributions, an identity link function and standardization of the input. However, in contrast to the weak models, the strong predictor employs Lasso regression to select only a minimal set of processes that are most informative of chronological age. The combination of processes with non-zero coefficients is considered to be predictive of age.

Importantly, the identification of cellular processes that are predictive of age requires assembling a training dataset in which the chronological age of each sample is a priori known. In this regard, both the weak and strong age predictors are not trained based on ages expressed in years. Instead, the age is transformed by a piecewise, approximately linear transformation defined as:

$$f ( x ) = \{ \begin{matrix}( ( log ( \frac{1}{71} - \frac{1}{101} ) \cdot log 25 ) - ( log ( \frac{1}{70} - \frac{1}{101} ) \cdot log 25 ) ) \cdot ( x - 70 ) , & x \leq 70 \\ 3 \cdot ( ( log ( \frac{1}{71} - \frac{1}{101} ) \cdot log 25 ) - ( log ( \frac{1}{70} - \frac{1}{101} ) \cdot log 25 ) ) \cdot ( x - 70 ) , & x > 70\end{matrix}$$

The function is a suitable transformation, since its inverse has the following closed form:

$${f}^{- 1} ( x ) = \{ \begin{matrix}\frac{- x + 70 \cdot log ( \frac{7070}{31} ) - 70 \cdot log ( \frac{7171}{30} )}{log ( \frac{7070}{31} ) - log ( \frac{7171}{30} )} , & x \leq 0 \\ \frac{- x + 210 \cdot log ( \frac{7070}{31} ) - 210 \cdot log ( \frac{7171}{30} )}{3 \cdot ( log ( \frac{7070}{31} ) - log ( \frac{7171}{30} ) )} , & x > 0\end{matrix}$$

### Training of Process-based Transcriptional Clocks

The transcriptomic clock introduced in this study resides on a set of cellular processes that are predictive of age, as described in the previous subsection. In particular, training of the clock follows a two-step process. First, for each process, the most predictive set of genes is selected by starting from a model with only an intercept, iteratively adding or removing a gene and scoring each iteration using Akaike's Information Criterion⁵¹ by performing linear regression between the current gene set and sample age. In total, 1000 iterations are performed for each process. Second, a GLM, implemented in the h2o v3.36.0.2 R package, is trained on all selected genes of all processes that were selected in the first step. More specifically, the GLM employs Gaussian distributions with an identity link function, lambda search and ridge regression on the standardized gene expression data. Since the number of genes selected in the first step may be larger than the number of training samples, an upper bound on the number of active predictors in the GLM was set to the number of training samples.

### Predicting the Age of Non-training Samples

Due to the variability in raw RNA-seq data, which is related to sequencing depth, library preparation and other experimental confounding factors, it is necessary to pre-process the training and non-training samples together to detect and correct batch effects. Therefore, for a set of new non-training samples, a common pre-processing pipeline was employed. First, training and non-training samples are merged into one matrix and TMM normalized using the ‘tmm’-function of the NOIseq v2.34.0 R package⁵³. Secondly, in case of significant batch effects visible in the first two principal components, batch correction is applied between the training and non-training samples. For the majority of datasets in this work, the naïve Removal of Unwanted Variance (RUV)⁵⁴ algorithm from the RUVnormalize v1.24.0 R package was employed. RUV requires the selection of an appropriate set of control genes that are expected to be uncorrelated to the variable of interest, e.g., age. Thus, appropriate controls were selected as having a low correlation with age in the training data (Pearson's r<0.01). Nevertheless, depending on the observed batch effects, other dataset-specific control genes should be included (see individual scripts for the batch correction methods and parameters employed for each dataset).

After normalization and batch correction, the transcriptional clock was trained on the training data as described in the previous section. The age of non-training samples was then predicted using the ‘h2o.predict’ function in the H2O R package on the trained model.

### Computing Process Activity Scores for Quantifying Age-Associated Functional Differences

Using the estimated coefficients of the transcriptomic clock, the activity of all eight age-associated processes was quantified in a sample by computing the scalar product of the model coefficients and the expression values of the corresponding genes. In case of multiple replicates, the activity scores of the same processes in different samples are aggregated into their arithmetic mean. Since RNA-seq data has been shown to be sensitive to the preprocessing pipeline employed to transform raw read counts, a reference process activity was computed for all samples of the training data. These reference activities define a range of values for each process that correspond to physiological aging and are employed to uniformly scale the activity scores of new samples. Due to the generalized linear model underlying the clock, lower process activity scores correspond to lower whereas higher values correspond to higher transcriptional age.

### Screening Library Construction

Q5 high-fidelity 2× master mix (NEB M0492S) was used to amplify all of the ORFs from their original vectors (Addgene or ORFeome) in order to add attB sites, the Kozak consensus sequence “GCCACC”, and the WT STOP codon. The amplified fragments were gel purified (QIAGEN 28506) and shuttled into pDONR221 (ThermoFisher 12536017) using the BP Clonase II enzyme mix (ThermoFisher 11789020). The reactions were transformed into 5-alpha competent E. coli (NEB C2987H), clones were picked and sequence confirmed using Sanger sequencing. The resulting plasmids were miniprepped (NEB T1010L) and reacted with a barcoded pool of destination vectors (PB-CT3G-ERP2-MG-BC) in a MegaGate reaction. After transforming into 5-alpha cells, clones were picked, sequence confirmed, and barcodes were assigned to specific ORFs. Final plasmids were miniprepped and used for nucleofection.

### Tissue Culture

NHDF lines were cultured at 37° C., 5% CO2, 5% 02 in fibroblast media (FM): low glucose DMEM (ThermoFisher 11885-084) supplemented with 15% FBS (GenClone 25-550) and 1% Penicillin-Streptomycin (ThermoFisher 15140122). When induced with Doxycycline, cells were switched to media made with Tet System Approved FBS (Takara 631367) to reduce background induction. Media was changed every other day.

For generating the overexpression lines, 200,000 cells were nucleofected with 50 fmol transposon and 50 fmol transposase (Super piggyBac Transposase-SystemBio PB210PA-1) or with 300 ng pmaxGFP using the P2 Primary Cell 4D Nucleofector kit (Lonza V4SP-2096) and the Lonza 4D-Nucleofector with the DS-150 program. Cells were recovered at room temperature for 45 min and then plated to a 24 well plate in 500 uL FM. The following day after nucleofection, dead cells were washed with PBS and media was replenished. 3-4 days post-nucleofection (depending on cell confluency) selection was started using 400 ng/mL Puromycin (ThermoFisher A1113802) in FM (PFM). Cells were selected and expanded at the same time, switching between FM and PFM every 4 days, until the pmaxGFP control reached 0% viability. At the 10 cm dish stage, the cells were frozen in FM with 5% DMSO and stored in liquid nitrogen until thaw. Due to cell line specific sensitivity to the nucleofection and selection process, as well as the increased toxicity of plasmids harboring larger genes, overexpression lines for all of the 95 genes in all three primary NHDF lines could not be generated.

When assayed, cells were thawed into 2 wells of a 6 well plate in FM, and switched to the Tet-free media the next day. 2 days after thaw, cells were harvested, counted, and seeded for RNA-Seq and flow cytometry. For RNA-Seq, each cell line was seeded into 2 wells of a 6 well plate at a density of 60,000 cells/well in FM supplemented with Doxycycline (1 ug/mL for the initial screen and 2 ug/mL for subsequent experiments). For flow cytometry, each line was seeded into 18 wells of a 24 well plate at a density of 10,000 cells/well in FM (the +Dox wells received Doxycycline at 1 μg/mL). 72 h post seeding, the cells were washed with PBS and either stained and harvested for flow cytometry or lysed using the Monarch DNA/RNA Protection Reagent (NEB T2011L) and stored at −80° C.

### RNA Sequencing

RNA was extracted from the cell lysates using the Monarch Total RNA Miniprep kit (NEB T2010S) and its quality was spot-checked for random samples using the Bioanalyzer High Sensitivity RNA Kit (Agilent 5067-1513). 100 ng of total RNA was quantified using the Qubit RNA High Sensitivity Assay (ThermoFisher Q32852) and used for library preparation with the NEBNext Ultra II Directional RNA Library Prep Kit for Illumina and the polyA mRNA workflow. The library quality was spot-checked for random samples using the Bioanalyzer High Sensitivity DNA Kit (5067-4626), and then all libraries were quantified using the Qubit dsDNA High Sensitivity Assay (ThermoFisher Q33230) and pooled together. RNA extraction, as well as library preparation, was performed on the same day for all samples that were sequenced together. Sequencing was performed by the Harvard Biopolymers Facility on an Illumina NextSeq or NovaSeq instrument.

### Flow Cytometry

Cells were stained for cellular senescence, mitochondrial membrane potential, and proteasome activity. For cellular senescence, cells were incubated with 100 nM Bafilomycin A1 (VWR 102513) for 2 h and with 33 nM C12FDG (5-Dodecanoylaminofluorescein Di-β-D-Galactopyranoside; ThermoFisher D2893) for 1 h. Mitochondrial membrane potential and proteasome activity were multiplexed by incubating cells with 20 nM or 40 nM (M65: [46, 55, 56, 57, 60, 64, 72, 77, 82, 94, 95]) TMRM (ThermoFisher M20036), 0.125× proteasome LLVY-R110 substrate, and 0.0625× assay buffer (Millipore Sigma MAK172) for 2 h. Samples were analyzed using either a Cytoflex LX or BD LSRfortessa instrument. Analysis was done using FlowJo (Version 10.8.1).

### In Vitro Scratch Assay

Cells from the M79 line harboring overexpression cassettes for SRSF1, OSKM, or mTagBFP2 were grown in 10 cm dishes and treated with doxycycline (1 ug/mL) for 3 days. Cells were subsequently harvested and seeded into 24 well plates at a density of 120,000 cells per well. The next morning, plates were scratched with a p200 pipette and washed with PBS. Plates were imaged using the Cellcyte live-cell imaging system (Cytena) with a 10× objective at an interval of 1.5 h.

Scratch images were stitched, processed, and analyzed as virtual stacks for each time course using Fiji. After cropping to the scratch area, the image backgrounds were subtracted using a 10-pixel rolling ball radius and contrast enhanced to 10% saturated pixels, normalized for all images in the stack. The Python package Bowhead v1.1.3 was used to identify the largest contiguous wound area in the images using a threshold of 0.5.

### Statistical Analysis

Statistical analyses were performed with R version 4.0.3, using two-tailed Student's t-tests, or a Z-score. All of the statistical tests performed are indicated in the figure legends. The data are presented as mean or individual points with box plots that show median and quartiles. Error bars represent standard error around the mean.

### Construct Sequences

SRSF1 sequences
>pAMP531_(PB-CT3G-ERP2-MG-BC-SRSF1)
(SEQ ID NO: 3)
ATCACCTCGAGTTTACTCCCTATCAGTGATAGAGAACGTATGAAGAGTTTACTCCCTATCAGTGATAGAGAACGT
ATGCAGACTTTACTCCCTATCAGTGATAGAGAACGTATAAGGAGTTTACTCCCTATCAGTGATAGAGAACGTATG
ACCAGTTTACTCCCTATCAGTGATAGAGAACGTATCTACAGTTTACTCCCTATCAGTGATAGAGAACGTATATCC
AGTTTACTCCCTATCAGTGATAGAGAACGTATAAGCTTGCCTATATAAGCAGAGCTCGTTTAGTGAACCGTCAGA
TCGCCTGGAGACGCCATCCACGCTGTTTTGACCTCCATAGAAGACACCGGGACCGATCCAGCCTCCGCGGTGGCG
GCATCGGTACACAAGTTTGTACAAAAAAGCAGGCTTAGCCACCATGTCGGGAGGTGGTGTGATTCGTGGCCCCGC
AGGGAACAACGATTGCCGCATCTACGTGGGTAACTTACCTCCAGACATCCGAACCAAGGACATTGAGGACGTGTT
CTACAAATACGGCGCTATCCGCGACATCGACCTCAAGAATCGCCGCGGGGGACCGCCCTTCGCCTTCGTTGAGTT
CGAGGACCCGCGAGACGCGGAAGACGCGGTGTATGGTCGCGACGGCTATGATTACGATGGGTACCGTCTGCGGGT
GGAGTTTCCTCGAAGCGGCCGTGGAACAGGCCGAGGCGGCGGCGGGGGTGGAGGTGGCGGAGCTCCCCGAGGTCG
CTATGGCCCCCCATCCAGGCGGTCTGAAAACAGAGTGGTTGTCTCTGGACTGCCTCCAAGTGGAAGTTGGCAGGA
TTTAAAGGATCACATGCGTGAAGCAGGTGATGTATGTTATGCTGATGTTTACCGAGATGGCACTGGTGTCGTGGA
GTTTGTACGGAAAGAAGATATGACCTATGCAGTTCGAAAACTGGATAACACTAAGTTTAGATCTCATGAGGGAGA
AACTGCCTACATCCGGGTTAAAGTTGATGGGCCCAGAAGTCCAAGTTATGGAAGATCTCGATCTCGAAGCCGTAG
TCGTAGCAGAAGCCGTAGCAGAAGCAACAGCAGGAGTCGCAGTTACTCCCCAAGGAGAAGCAGAGGATCACCACG
CTATTCTCCCCGTCATAGCAGATCTCGCTCTCGTACATAATACCCAGCTTTCTTGTACAAAGTGGTATACCCATA
CGATGTTCCAGATTACGCTTAGTAATGAGAAATACTCAGAAGATGTCACTCACCATAGTCCGCAGTGAAAGATAA
ATGATCGCTGCGTTTGATGAGTTTGTGTTTTAGAGCTAGAAATAGCAAGTAGGCCACTGAGCAAGAACTATGAAT
GAGATCTTTAGACATATTGCGGCCGCTTTCTCCGCGATCCAGACATGATAAGATACATTGATGAGTTTGGACAAA
CCAAAACTAGAATGCAGTGAAAAAAATGCCTTATTTGTGAAATTTGTGATGCTATTGCCTTATTTGTAACCATTA
TAAGCTGCAATAAACAAGTTATCTATAACAAGAAAATATATATATAATAAGTTATCACGTAAGTAGAACATGAAA
TAACAATATAATTATCGTATGAGTTAAATCTTAAAAGTCACGTAAAAGATAATCATGCGTCATTTTGACTCACGC
GGTCGTTATAGTTCAAAATCAGTGACACTTACCGCATTGACAAGCACGCCTCACGGGAGCTCCAAGCGGCGACTG
AGATGTCCTAAATGCACAGCGACGGATTCGCGCTATTTAGAAAGAGAGAGCAATATTTCAAGAATGCATGCGTCA
ATTTTACGCAGACTATCTTTCTAGGGTTAAGAATTCACTGGCCGTCGTTTTACAACGTCGTGACTGGGAAAACCC
TGGCGTTACCCAACTTAATCGCCTTGCAGCACATCCCCCTTTCGCCAGCTGGCGTAATAGCGAAGAGGCCCGCAC
CGATCGCCCTTCCCAACAGTTGCGCAGCCTGAATGGCGAATGGCGCCTGATGCGGTATTTTCTCCTTACGCATCT
GTGCGGTATTTCACACCGCATATGGTGCACTCTCAGTACAATCTGCTCTGATGCCGCATAGTTAAGCCAGCCCCG
ACACCCGCCAACACCCGCTGACGCGCCCTGACGGGCTTGTCTGCTCCCGGCATCCGCTTACAGACAAGCTGTGAC
CATGTCTCCGGGAGCTGCATGTGTCAGAGGTTTTCACCGTCATCACCGAAACACGCGAGTTCGAAAGGGCCTCGT
GATACGCCTATTTTTATAGGTTAATGTCATGATAATAATGGTTTCTTAGACGTCAGGTGGCACTTTTCGGGGAAA
TGTGCGCGGAACCCCTATTTGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGTGTCGCCCTTATTCCCTTTTT
TGCGGCATTTTGCCTTCCTGTTTTTGCTCACCCAGAAACGCTGGTGAAAGTAAAAGATGCTGAAGATCAGTTGGG
TGCACGAGTGGGTTACATCGAACTGGATCTCAACAGCGGTAAGATCCTTGAGAGTTTTCGCCCCGAAGAACGTTT
TCCAATGATGAGCACTTTTAAAGTTCTGCTATGTGGCGCGGTATTATCCCGTATTGACGCCGGGCAAGAGCAACT
CGGTCGCCGCATACACTATTCTCAGAATGACTTGGTTGAGTACTCACCAGTCACAGAAAAGCATCTTACGGATGG
CATGACAGTAAGAGAATTATGCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAGCAATGGCAACAACGTTGCG
CAAACTATTAACTGGCGAACTACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAATCTGGAGCCGGTGAGCGTGG
TTCACGCGGTATCATTGCAGCACTGGGGCCAGATGGTAAGCCCTCCCGTATCGTAGTTATCTACACGACGGGGAG
TCAGGCAACTATGGATGAACGAAATAGACAGATCGCTGAGATAGGTGCCTCACTGATTAAGCATTGGTAACTGTC
AGACCAAGTTTACTCATATATACTTTAGATTGATTTAAAACTTCATTTTTAATTTAAAAGGATCTAGGTGAAGAT
CCTTTTTGATAATCTCATGACCAAAATCCCTTAACGTGAGTTTTCGTTCCACTGAGCGTCAGACCCCGTAGAAAA
GATCAAAGGATCTTCTTGAGATCCTTTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTTGGAGCGAACGAC
CTACACCGAACTGAGATACCTACAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAGGGGGAAACGCCTGGTATCTTTA
TAGTCCTGTCGGGTTTCGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCGCTCGCCGCAGCCGAACGAC
CGAGCGCAGCGAGTCAGTGAGCGAGGAAGCGGATGAGCGCCCAATACGCAAACCGCCTCTCACCGCGCGTTGGCC
GATTCATTAATGCAGCTGGCACGACAGGTTTCCCGACTGGAAAGCGGGCAGTGAGCGCAACGCAAACCGCCTCTC
CCCGCGCGTTGGCCGATTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCGGCTCGTATGTTGTGT
GGAATTGTGAGCGGATAACAATTTCACACAGGAAACAGCTATGACCATGATTACGCCAAGGTCGACTTAACCCTA
GAAAGATAATCATATTGTGACGTACGTTAAAGATAATCATGCGTAAAATTGACGCATGTGTTTTATCGGTCTGTA
TATCGAGGTTTATTTATTAATTTGAATAGATATTAAGTTTTATTATATTTACACTTACATACTAATAATAAATTC
AACAAACAATTTATTTATGTTTATTTATTTATTAAAAAAAAACAAAAACTCAAAATTTCTTCTATAAAGTAACAA
AACTTTTAGCAGTGAAAAAAATGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGTGTGGGAGGTTTTT
TAAAGCAAGTAAAACCTCTACAAATGTGGTATGGCTGATTATGATCCTCTGGAGATTCATGCGCCAGGTTTTCTG
GTCATGCACCACGTTCGTGGGCCCTCAGGAACCTCCACGTCAGCGGTAACTGTGAAGCCCAGTCGCTCGTAAAAA
GGAAGATTCCGGGGAGCACTGGTTTCGAGGAACGCCGGCACGCCAGCCCGTTCCGCGGCCTCAACACCAGGAAGA
ACGACAGCGCTTCCCAAACCTTTACCTTGGTGATCTGGGCTGACACCGACAGTGGCAAGAAACCACGCAGGCTCC
TTTGGTCTATGAGGAGCCAGCAAGCCCTCCATTTGCTGTTGTGCCGCCAACCTACTCCCTGACAGCTCGGCCATC
CGCGGACCTATTTCCGCGAACACTGCGCCGGCTTCGACGCTTTCCGGAGTAGTCCAGACCGCAACTGCGGCACCA
TCGTCCGCAACCCAAACTTTACCAATGTCCAGACCCACGCGCGTAAGAAACAGCTCTTGCAACTCAGTTACCCTC
TCTATGTGTCTGTCCGGATCTACGGTATGGCGGGTCGCTGGATAATCTGCAAATGCTGCGGCAAGTGTTCGGACA
GCCCGTGGTACATCGTCACGGGTAGCGAGCCTAACAGTGGGCTTATATTCCGTCATGGGGCCGGGGTTCTCCTCC
ACGTCGCCGGCCTGCTTCAGCAGGCTGAAGTTGGTGGCGCCGCTGCCCCCGGGGAGCATGTCAAGGTCAAAATCG
TCAAGAGCGTCAGCAGGCAGCATATCAAGGTCAAAGTCGTCAAGGGCATCGGCTGGGAGCATGTCTAAGTCAAAA
TCGTCAAGGGCGTCGGTCGGCCCGCCGCTTTCGCACTTTAGCTGTTTCTCCAGGCCACATATGATTAGTTCCAGG
CCGAAAAGGAAGGCAGGTTCGGCTCCCTGCCGGTCGAACAGCTCAATTGCTTGTTTCAGAAGTGGGGGCATAGAA
TCGGTGGTAGGTGTCTCTCTTTCCTCTTTTGCTACTTGATGCTCCTGTTCCTCCAATACGCAGCCCAGTGTAAAG
TGGCCCACGGCGGACAGAGCGTACAGTGCGTTCTCCAGGGAGAAGCCTTGCTGACACAGGAACGCGAGCTGATTT
TCCAGGGTTTCGTACTGTTTCTCTGTTGGGCGGGTGCCGAGATGCACTTTAGCCCCGTCACGATGTGAGAGGAGA
GCACAGCGGTATGACTTGGCGTTGTTCCGCAGAAAGTCTTGCCATGACTCGCCTTCCAGGGGGCAGGAGTGGGTA
TGATGCCTGTCCAGCATCTCGATTGGCAGGGCATCGAGCAGGGCCCGCTTGTTCTTCACGTGCCAGTACAGGGTA
GGCTGCTCAACTCCCAGCTTTTGAGCGAGTTTCCTTGTCGTCAGGCCTTCGATACCGACTCCATTGAGTAATTCC
AGAGCAGAGTTTATGACTTTGCTCTTGTCCAGTCTAGACATCTTATCGTCATCGTCTTTGTAATCCATGGTGGCG
GATCCCGCGTCACGACACCTGTGTTCTGGCGGCAAACCCGTTGCGAAAAAGAACGTTCACGGCGACTACTGCACT
TATATACGGTTCTCCCCCACCCTCGGGAAAAAGGCGGAGCCAGTACACGACATCACTTTCCCAGTTTACCCCGCG
CCACCTTCTCTAGGCACCGGTTCAATTGCCGACCCCTCCCCCCAACTTCTCGGGGACTGTGGGCGATGTGCGCTC
TGCCCACTGACGGGCACCGGAGCCACTCGAGTGGAATT
>SRSF1_full
(SEQ ID NO: 4)
GCAGTGAAAAAAATGCTTTATTTGTGAAATTTGTGATGCTATTGCTTTATTTGTAACCATTATAAGCTGCAATAA
ACAAGTTAACAACAACAATTGCATTCATTTTATGTTTCAGGTTCAGGGGGAGGTGTGGGAGGTTTTTTAAAGCAA
GTAAAACCTCTACAAATGTGGTATGGCTGATTATGATCCTCTGGAGATTCATGCGCCAGGTTTTCTGGTCATGCA
CCACGTTCGTGGGCCCTCAGGAACCTCCACGTCAGCGGTAACTGTGAAGCCCAGTCGCTCGTAAAAAGGAAGATT
CCGGGGAGCACTGGTTTCGAGGAACGCCGGCACGCCAGCCCGTTCCGCGGCCTCAACACCAGGAAGAACGACAGC
GCTTCCCAAACCTTTACCTTGGTGATCTGGGCTGACACCGACAGTGGCAAGAAACCACGCAGGCTCCTTTGGTCT
ATGAGGAGCCAGCAAGCCCTCCATTTGCTGTTGTGCCGCCAACCTACTCCCTGACAGCTCGGCCATCCGCGGACC
TATTTCCGCGAACACTGCGCCGGCTTCGACGCTTTCCGGAGTAGTCCAGACCGCAACTGCGGCACCATCGTCCGC
AACCCAAACTTTACCAATGTCCAGACCCACGCGCGTAAGAAACAGCTCTTGCAACTCAGTTACCCTCTCTATGTG
TCTGTCCGGATCTACGGTATGGCGGGTCGCTGGATAATCTGCAAATGCTGCGGCAAGTGTTCGGACAGCCCGTGG
TACATCGTCACGGGTAGCGAGCCTAACAGTGGGCTTATATTCCGTCATGGGGCCGGGGTTCTCCTCCACGTCGCC
GGCCTGCTTCAGCAGGCTGAAGTTGGTGGCGCCGCTGCCCCCGGGGAGCATGTCAAGGTCAAAATCGTCAAGAGC
GTCAGCAGGCAGCATATCAAGGTCAAAGTCGTCAAGGGCATCGGCTGGGAGCATGTCTAAGTCAAAATCGTCAAG
GGCGTCGGTCGGCCCGCCGCTTTCGCACTTTAGCTGTTTCTCCAGGCCACATATGATTAGTTCCAGGCCGAAAAG
GAAGGCAGGTTCGGCTCCCTGCCGGTCGAACAGCTCAATTGCTTGTTTCAGAAGTGGGGGCATAGAATCGGTGGT
AGGTGTCTCTCTTTCCTCTTTTGCTACTTGATGCTCCTGTTCCTCCAATACGCAGCCCAGTGTAAAGTGGCCCAC
GGCGGACAGAGCGTACAGTGCGTTCTCCAGGGAGAAGCCTTGCTGACACAGGAACGCGAGCTGATTTTCCAGGGT
TTCGTACTGTTTCTCTGTTGGGCGGGTGCCGAGATGCACTTTAGCCCCGTCACGATGTGAGAGGAGAGCACAGCG
GTATGACTTGGCGTTGTTCCGCAGAAAGTCTTGCCATGACTCGCCTTCCAGGGGGCAGGAGTGGGTATGATGCCT
GTCCAGCATCTCGATTGGCAGGGCATCGAGCAGGGCCCGCTTGTTCTTCACGTGCCAGTACAGGGTAGGCTGCTC
AACTCCCAGCTTTTGAGCGAGTTTCCTTGTCGTCAGGCCTTCGATACCGACTCCATTGAGTAATTCCAGAGCAGA
GTTTATGACTTTGCTCTTGTCCAGTCTAGACATCTTATCGTCATCGTCTTTGTAATCCATGGTGGCGGATCCCGC
GTCACGACACCTGTGTTCTGGCGGCAAACCCGTTGCGAAAAAGAACGTTCACGGCGACTACTGCACTTATATACG
GTTCTCCCCCACCCTCGGGAAAAAGGCGGAGCCAGTACACGACATCACTTTCCCAGTTTACCCCGCGCCACCTTC
TCTAGGCACCGGTTCAATTGCCGACCCCTCCCCCCAACTTCTCGGGGACTGTGGGCGATGTGCGCTCTGCCCACT
GACGGGCACCGGAGCCACTCGAGTGGAATTATCACCTCGAGTTTACTCCCTATCAGTGATAGAGAACGTATGAAG
AGTTTACTCCCTATCAGTGATAGAGAACGTATGCAGACTTTACTCCCTATCAGTGATAGAGAACGTATAAGGAGT
TTACTCCCTATCAGTGATAGAGAACGTATGACCAGTTTACTCCCTATCAGTGATAGAGAACGTATCTACAGTTTA
CTCCCTATCAGTGATAGAGAACGTATATCCAGTTTACTCCCTATCAGTGATAGAGAACGTATAAGCTTGCCTATA
TAAGCAGAGCTCGTTTAGTGAACCGTCAGATCGCCTGGAGACGCCATCCACGCTGTTTTGACCTCCATAGAAGAC
ACCGGGACCGATCCAGCCTCCGCGGTGGCGGCATCGGTACACAAGTTTGTACAAAAAAGCAGGCTTAGCCACCAT
GTCGGGAGGTGGTGTGATTCGTGGCCCCGCAGGGAACAACGATTGCCGCATCTACGTGGGTAACTTACCTCCAGA
CATCCGAACCAAGGACATTGAGGACGTGTTCTACAAATACGGCGCTATCCGCGACATCGACCTCAAGAATCGCCG
CGGGGGACCGCCCTTCGCCTTCGTTGAGTTCGAGGACCCGCGAGACGCGGAAGACGCGGTGTATGGTCGCGACGG
CTATGATTACGATGGGTACCGTCTGCGGGTGGAGTTTCCTCGAAGCGGCCGTGGAACAGGCCGAGGCGGCGGCGG
GGGTGGAGGTGGCGGAGCTCCCCGAGGTCGCTATGGCCCCCCATCCAGGCGGTCTGAAAACAGAGTGGTTGTCTC
TGGACTGCCTCCAAGTGGAAGTTGGCAGGATTTAAAGGATCACATGCGTGAAGCAGGTGATGTATGTTATGCTGA
TGTTTACCGAGATGGCACTGGTGTCGTGGAGTTTGTACGGAAAGAAGATATGACCTATGCAGTTCGAAAACTGGA
TAACACTAAGTTTAGATCTCATGAGGGAGAAACTGCCTACATCCGGGTTAAAGTTGATGGGCCCAGAAGTCCAAG
TTATGGAAGATCTCGATCTCGAAGCCGTAGTCGTAGCAGAAGCCGTAGCAGAAGCAACAGCAGGAGTCGCAGTTA
CTCCCCAAGGAGAAGCAGAGGATCACCACGCTATTCTCCCCGTCATAGCAGATCTCGCTCTCGTACATAATACCC
AGCTTTCTTGTACAAAGTGGTATACCCATACGATGTTCCAGATTACGCTTAGTAATGAGAAATACTCAGAAGATG
TCACTCACCATAGTCCGCAGTGAAAGATAAATGATCGCTGCGTTTGATGAGTTTGTGTTTTAGAGCTAGAAATAG
CAAGTAGGCCACTGAGCAAGAACTATGAATGAGATCTTTAGACATATTGCGGCCGCTTTCTCCGCGATCCAGACA
TGATAAGATACATTGATGAGTTTGGACAAACCAAAACTAGAATGCAGTGAAAAAAATGCCTTATTTGTGAAATTT
GTGATGCTATTGCCTTATTTGTAACCATTATAAGCTGCAATAAACAAGTT

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A method of inducing cellular rejuvenation of a cell comprising contacting the cell with an effective amount of a Serine and Arginine Rich Splicing Factor 1 (SRSF1) protein or a nucleic acid encoding the SRSF1 protein.

2. A method of inducing cellular rejuvenation of a cell in a subject comprising administering to the subject an effective amount of a SRSF1 protein or a nucleic acid encoding the SRSF1 protein.

3. The method of claim 1 or 2, wherein the effective amount is sufficient to decrease cellular senescence of the cell, relative to a control.

4. The method of claim 3, wherein the effective amount is sufficient to decrease cellular senescence of the cell by at least 20%, at least 30%, at least 40%, or at least 50%.

5. The method of claim 4, wherein the effective amount is sufficient to decrease cellular senescence of the cell by at least 50%.

6. The method of any one of claims 3-5, wherein the effective amount is sufficient to decrease senescence-associated β-galactosidase activity of the cell, relative to a control.

7. The method of claim 6, wherein the effective amount is sufficient to decrease the senescence-associated β-galactosidase activity of the cell by at least 25%, at least 30%, at least 35%, at least 40%, at least 45%, or at least 50%.

8. The method of any one of claims 3-7, wherein the effective amount is sufficient to decrease proteasomal activity of the cell, relative to a control.

9. The method of claim 8, wherein the effective amount is sufficient to decrease the proteasomal activity of the cell by at least 25%, at least 30%, at least 40%, at least 45%, or at least 50%.

10. The method of any one of the preceding claims, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.

11. The method of claim 10, wherein the effective amount is sufficient to induce an average cellular rejuvenation of at least 25 years.

12. The method of any one of the preceding claims, wherein the cells are selected from fibroblasts, hematopoietic stem cells, endothelial cells, chondrocytes, skeletal muscle stem cells, keratinocytes, mesenchymal stem cells and corneal epithelial cells.

13. The method of claim 12, wherein the cells are fibroblasts.

14. The method of claim 13, wherein the fibroblasts are human dermal fibroblasts.

15. The method of any one of the preceding claims, wherein the nucleic acid comprises a heterologous promoter operably linked to the open reading frame.

16. The method of claim 15, wherein the heterologous promoter is an inducible promoter.

17. The method of any one of the preceding claims, comprising delivering to cells the SRSF1 protein.

18. The method of any one of the preceding claims, comprising delivering to cells the nucleic acid comprising an open reading frame encoding the SRSF1 protein.

19. The method of any one of the preceding claims, wherein the nucleic acid is delivered on a non-viral vector or a viral vector.

20. The method of any one of the preceding claims, wherein the contacting comprises transfecting the cells.

21. A method of inducing cellular rejuvenation of a cell comprising overexpressing in the cell an effective amount of a SRSF1 protein.

22. The method of claim 21, wherein the method comprises activating expression or activity of endogenous SRSF1 protein at a level that is higher than a baseline level.

23. A cell comprising an engineered nucleic acid encoding a Serine and Arginine Rich Splicing Factor 1 (SRSF1) protein.

24. The cell of claim 23, wherein the cell is a fibroblast.

25. The cell of claim 24, wherein the fibroblast is a human dermal fibroblast.

26. The cell of claim 23, wherein the cell is a stem cell.

27. The cell of claim 26, wherein the stem cell is selected from hematopoietic stem cells, skeletal muscle stem cells, and mesenchymal stem cells.

28. The cell of claim 26, wherein the stem cell is a human induced pluripotent stem cell.

29. The cell of claim 23, wherein the cell is selected from endothelial cells, chondrocytes, keratinocytes, and corneal epithelial cells.

30. The cell of any one of claims 23-29, wherein the cell expresses SRSF1 at a level that is higher than a baseline level.
